(19) United States
(12) Patent Application Publication
(10) Pub. No.: US 2006/0128695 A1
(43) Pub. Date: Jun. 15, 2006

(54) Cyclic nucleotide phosphodiesterase inhibitors, preparation and uses

(75) Inventors: Jean-Jacques Bourguignon, Hipsheim (FR); Claire Lugnier, Strasbourg (FR); Mustapha Abarghaz, Strasbourg (FR); Yan Lagouge, Strasbourg (FR); Patrick Wagner, Brumath (FR); Cesare Mondadori, Reinach (CH); Jean-Paul Macher, Bergholtz-Zell (FR); Dominique Schultz, Illkirch (FR); Pierre Raboisson, Exton, PA (US)

Correspondence Address: NIXON & VANDERHYE, PC, 901 NORTH GLEBE ROAD, 11TH FLOOR, ARLINGTON, VA, 22203, US

(73) Assignees: NEURO3D, Mulhouse (FR); UNIVERSITE LOUIS PASTEUR, Strasbourg (FR)

(21) Appl. No.: 10/533,157

(22) PCT Filed: Oct. 30, 2003
(86) PCT No.: PCT/FR03/03247

§ 371 Date: Dec. 7, 2005

Related U.S. Application Data
(60) Provisional application No. 60/455,874, filed on Mar. 20, 2003.

(30) Foreign Application Priority Data
Oct. 30, 2002 (FR) 02/13607

(51) Int. Cl. A61K 31/5513; C07D 403/02; C07D 243/12

(52) U.S. Cl. 514/221; 540/504

(57) ABSTRACT

The invention concerns the use of PDE2 inhibitors for treating disorders of the central and peripheral nervous system, a method for therapeutic treatment by administering to an animal said inhibitors. More specifically, the invention concerns novel benzodiazepinone derivatives and their uses in therapeutics more particularly for treating pathologies involving activity of a cyclic nucleotide phosphodiesterase type 2. The invention also concerns methods for preparing same and novel synthesis intermediates.

The invention concerns the use of PDE2 inhibitors for treating disorders of the central and peripheral nervous system, a method for therapeutic treatment by administering to an animal said inhibitors. More specifically, the invention concerns novel benzodiazepine derivatives and their uses in therapeutics more particularly for treating pathologies involving activity of a cyclic nucleotide phosphodiesterase. The invention also concerns methods for preparing same and novel synthesis intermediates.

The compounds whose synthesis is described in the present invention are novel and possess very interesting pharmacological properties: they are inhibitors of cyclic nucleotide phosphodiesterases and more particularly of cGS-PDE (cGMP-Stimulated PDEs or phosphodiesterase type 2 (PDE2) and, as such, they have very interesting therapeutic applications.

The functions of most tissues are modulated by endogenous substances such as hormones, transmitters, etc. or by exogenous substances. The biological effect of some of these substances is transmitted inside the cell by enzymatic effectors, such as adenylate cyclase or guanylate cyclase. Stimulation of said enzymes results in an elevation of intracellular levels of cyclic AMP (cAMP) or cyclic GMP (cGMP), second messengers involved in regulating many cellular activities. These cyclic nucleotides are degraded by a family of enzymes—the phosphodiesterases (PDE)—comprising at least seven groups.

One of them, PDE2, hydrolyzes both cAMP and cGMP and can be activated by cGMP. In physiological conditions PDE2 responds to high cGMP concentrations by increasing the hydrolysis of cAMP. This group is called PDE2 and is present in many tissues (adipocytes, adrenals, brain, heart, liver, lung, blood vessels, etc.). PDE2 inhibitors are capable of increasing or maintaining intracellular levels of both cAMP and cGMP and as such, find uses in the treatment of various pathologies.

The applicant has now demonstrated that certain benzodiazepines have inhibitory effects on cyclic nucleotide phosphodiesterases, particularly inhibition of PDE2. The invention also describes novel compounds exhibiting potent inhibitory activity towards PDE2, and preferentially displaying an excellent selectivity profile relative to other PDE isoforms, in particular a weak action on PDE3. Said selectivity can also extend to other enzymes, such as adenosine deaminase. Thus, the invention also describes novel compounds having potent inhibitory activity towards PDE2, and preferentially displaying an excellent selectivity profile on PDE2 in comparison with adenosine deaminase. Moreover, preferred compounds according to the invention have important central effects (anticonvulsant, anxiolytic, sedative, antidepressant) or peripheral effects (antirheumatismal, against auto-inflammatory diseases, against age-related liver dysfunction), and advantageously are devoid of memory impairing effects.

The invention therefore has as a first object the use of at least one phosphodiesterase 2 inhibitor for preparing a pharmaceutical composition for treating pathologies of the nervous system (central and peripheral), particularly central.

More specifically, the pathologies are those due to a deregulation of the function of a neurotransmitter or a deficiency in the release of a neurotransmitter (eg., dopamine, norepinephrine, acetylcholine, etc.), in particular dopamine, such as more specifically a pathology selected in the group consisting of depression, schizophrenia, anxiety, bipolar disorder, attention deficit disorders, Sleep disorders, OCD—obsessive compulsive disorder, fibromyalgia, Tourette's syndrome, pharmacodependence (to drugs, medication, alcohol, etc.), epilepsy, Alzheimer's disease, Parkinson's disease, amyotrophic lateral sclerosis, multiple sclerosis, obesity and Lewy body dementia.

PDE2 inhibitors can also be used according to the invention for treating other disorders involving the peripheral nervous system and peripheral organs in general, in particular pathologies of the type reduced natriuria, acute renal failure, hepatic dysfunction, acute hepatic failure, in particular due to age, and pathologies due to or involving dysfunctions of prolactin secretion, such as restless legs syndrome, rheumatismal, allergic or auto-inflammatory disorders, such as rheumatoid arthritis, rhinitis and asthma.

A particular object of the invention is therefore based on the use of PDE2 inhibitors for preparing a medicament for treating central or peripheral nervous system disorders, chronic or acute, or peripheral use of said inhibitors as vasoconstrictors.

According to a particular object of the invention, the PDE2 inhibitors are used to treat anxiety, depression or schizophrenia.

Inhibitors of the activity or the expression of type PDE2 phosphodiesterase which are particularly useful according to the invention are compounds which have selective PDE2 inhibitory activity, that is to say, they have less inhibitory activity towards other phosphodiesterases and particularly PDE1, PDE3, PDE4 and PDE5. Some of the PDE2 inhibitors are selected in particular in the scope of the invention for their selective inhibition of PDE2 relative to adenosine deaminase, meaning that they have more potent inhibitory activity for PDE2 than for adenosine deaminase.

Preferably, the PDE2 inhibitors used in the invention can be selected in the group consisting of 1,4-benzodiazepine derivatives.

In this context, the invention also describes novel compounds having potent PDE2 inhibitory activity. More particularly, the invention thus has as object compounds represented by general formula (I) 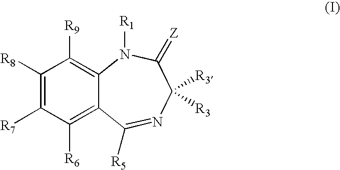
in which:
Z represents an oxygen, sulfur atom or a NR₂ group,
R₁ is the hydrogen atom, a (C₁-C₆) alkyl group, a (C₆-C₁₈) aryl group or a (C₁-C₆)alkyl(C₆-C₁₈)aryl or (C₆-C₁₈)aryl(C₁-C₄)alkyl group,
R₂ is a hydrogen atom, a (C₁-C₆) alkyl group, a (C₆-C₁₈) aryl group or a (C₁-C₆)alkyl(C₆-C₁₈)aryl or (C₆-C₁₈)aryl(C₁-C₄)alkyl group,
R₁ and R₂ taken together can optionally form a linear or branched hydrocarbon chain having from 2 to 6 carbon atoms, possibly containing one or several other double bonds and/or possibly interrupted by an oxygen, sulfur or nitrogen atom,
R₃ and R_{3′}, which are the same or different, represent the hydrogen atom, a (C₁-C₁₂) alkyl, (C₃-C₆) cycloalkyl, (C₆-C₁₈) aryl, (C₆-C₁₈)aryl(C₁-C₄)alkyl, (C₁-C₁₂)alkyl(C₆-C₁₈)aryl group or a (C₅-C₁₈) heterocycle, aromatic or not, containing 1 to 3 heteroatoms, a NO₂, CF₃, CN, NR′R″, SR′, OR′, COOR′, CONR′R″ or NHCOR′R″ group, R′ and R″, independently of each other, being selected in the group consisting of the hydrogen atom, a (C₁-C₆) alkyl, (C₃-C₆) cycloalkyl, (C₆-C₁₂) aryl group, and a (C₅-C₁₂) heterocycle, aromatic or not, containing 1 to 3 heteroatoms;
R₅ represents a phenyl group substituted at least in position 3, a naphthyl group, a (C₅-C₁₈) heterocycle, aromatic or not, containing 1 to 3 heteroatoms, selected in the group consisting of the pyridyl, isoquinolyl, quinolyl and piperazinyl group, provided that when R₅ is a naphthyl group substituted in position 6, then the latter is not attached to the rest of the molecule in position 2, or when R₅ is a pyridyl group, then it is not attached to the rest of the molecule in position 4, or when R₅ is a tetrahydro 1,2,3,4-isoquinolyl group, then it is not attached to the rest of the molecule in position 2, when R₅ represents a phenyl group substituted at least in position 3, said substituent being selected in the group consisting of: an alkyl, halogenoalkyl, cycloalkyl, alkenyl, alkynyl, aralkyl, aryl, heterocycle, heterocycloalkyl group, a OH, ═O, NO₂, NH₂, CN, CF₃, COR′, COOR′, (C₁-C₆)alkoxy, (di)(C₁-C₆)alkylamino, NHCOR′, CONR′R″ group, in which R′ and R″ are defined as hereinabove, CHO, CONH2, phenyl optionally substituted, in particular by an acetyl group, by a halogen atom (Cl), by a CONH2 group or by a CN group, prop-1-ynyl optionally substituted, in particular by a benzyloxy or tert-butyl carbamate group, hex-1-ynyl optionally substituted, in particular by a CN or NH2 group, pentyl optionally substituted, in particular by a CONH2, hexyl, piperidinyl optionally substituted, in particular by a prop-1-ynyl, benzylaminomethyl, acetamide (CH3CONH), aminomethyl, NH2CS—, 4-phenyl-1, 3-thiazol-2-yl, —-CONHBenzyl, —COOEthyl, —CONHiPropyl, —CONH—(CH2)n—CONH2 (n representing a whole number from 1 to 6), —CONR′R″, with R′ and R″, which are the same or different, representing a C1-C6 alkyl group or a hydrogen atom, -(4-benzylpyperazin-1-yl)carbonyl, —CONH—(CH2)n-phenyl (n representing a whole number from 1 to 6), imidazolyl, piperazinyl optionally substituted, in particular by a phenyl group,
R₇ and R₈, independently of each other, are selected in the group consisting of the hydrogen atom, a halogen atom or a OR₁₀ group, in which R₁₀ represents a hydrogen atom, a (C₁-C₆) alkyl, (C₃-C₆) cycloalkyl, (C₆-C₁₂) aryl group, or a (C₅-C₁₂) heterocycle, aromatic or not, comprising 1 to 3 heteroatoms, at least one of the groups R₇ and R₈ representing a OR₁₀ group such as defined hereinabove,
R₆ and R₉, independently of each other, are selected in the group consisting of the hydrogen atom, a halogen atom, an alkyl, cycloalkyl, alkenyl, alkynyl group, an aryl, aralkyl, heterocycle, heterocycloalkyl group and a OR₁₀ group, R₁₀ being such as defined hereinabove,
   the alkyl, cycloalkyl, alkenyl, alkynyl, aralkyl, aryl, phenyl, naphthyl, heterocycle, heterocycloalkyl group or the hydrocarbon chain defined earlier being optionally substituted by one or more substituents, which are the same or different, preferably selected in the group consisting of a halogen atom, an alkyl, halogenoalkyl, cycloalkyl, alkenyl, alkynyl, aralkyl, aryl, heterocycle, heterocycloalkyl group, a OH, ═O, NO₂, NH₂, CN, CF₃, COR′, COOR′, (C₁-C₆)alkoxy, (di)(C₁-C₆)alkylamino, NHCOR′ and CONR′R″ group, in which R′ and R″ are such as defined hereinabove, the substituents also being optionally substituted,
   and the salts of compounds represented by formula (I),
   with the exception of compounds represented by formula (I) in which
R1 is an alkyl group, R3 and R′3 are hydrogen atoms, R6 and R9 are hydrogen atoms, R5 is a phenyl group substituted at least in position 3 by a methoxy group,
R1 is an alkyl group or a hydrogen atom, R3 and R′3 are hydrogen atoms, R6 and R9 are hydrogen atoms, R5 is a phenyl group substituted only in position 3 by a chlorine or bromine atom,
R1 is an alkyl group, R3 and R′3 are hydrogen atoms, R6 and R9 are hydrogen atoms, R5 is a phenyl group substituted at least in position 3 by a CH2OH group,
R1 is a hydrogen atom, R3 and R′3 are hydrogen atoms, R6 and R9 are hydrogen atoms, R5 is a phenyl group substituted only in position 3 by a CF3 group,
R1 is an alkyl group, R3 and R′3 are hydrogen atoms, R6 and R9 are hydrogen atoms, R5 is a phenyl group substituted in positions 3 and 5 by a CF3 group,
R1 is an alkyl group, R3 and R′3 are hydrogen atoms, R6 and R9 are hydrogen atoms, R7 and R8 are methoxy groups, R5 is a phenyl group substituted in position 3 by a phenyl group,
R1 is an alkyl group, R3 and R′3 are hydrogen atoms, R6 and R9 are hydrogen atoms, R7 and R8 are methoxy groups, R5 is a phenyl group substituted in position 3 by a phenylethynyl group.

The invention also concerns pharmaceutical compositions comprising one or more compounds represented by general formula (I) such as defined hereinabove, and a pharmaceutically acceptable vehicle or excipient.

The invention further concerns the use of compounds represented by general formula (I) such as defined hereinabove for preparing a pharmaceutical composition intended for the inhibition of a cyclic nucleotide phosphodiesterase, in particular phosphodiesterase 2 (PDE2). More particularly, the invention concerns the use of the above compounds for treating pathologies involving a deregulation of intracellular levels of cyclic AMP and/or cyclic GMP.

In the spirit of the invention, the term “alkyl” designates a linear or branched hydrocarbon group advantageously containing from 1 to 12 carbon atoms, such as methyl, ethyl, propyl, isopropyl, butyl, isobutyl, tert-butyl, pentyl, neopentyl, n-hexyl, n-decyl, n-dodecyl, etc. C₁-C₆ groups are preferred. The alkyl groups may be substituted by an aryl group such as defined hereinbelow, in which case it is called an arylalkyl (or aralkyl) group. Benzyl and phenethyl are specific examples of arylalkyl groups.

The term “cycloalkyl” denotes a cyclic hydrocarbon system, which may advantageously contain from 3 to 6 carbon atoms and be mono- or poly-cyclic. Examples include cyclopropyl and cyclohexyl groups in particular.

“Aryl” groups are mono-, bi- or tri-cyclic aromatic hydrocarbon systems, preferably monocyclic or bicyclic aromatic hydrocarbon systems containing from 6 to 18 carbon atoms, even more preferably 6 carbon atoms. Examples include phenyl, naphthyl and biphenyl groups.

“Heterocycle” groups denote hydrocarbon systems, aromatic or not, containing one or more cyclic heteroatoms. Preferably they are cyclic hydrocarbon systems containing from 5 to 18 carbon atoms and one or more cyclic heteroatoms, particularly from 1 to 3 or 4 cyclic heteroatoms chosen from among N, O and S. Preferred aromatic heterocyclic groups (heteroaryls) include in particular thienyl, benzothienyl, benzofuryl, pyridyl, pyrimidinyl, pyridazinyl, isoquinolyl, quinolyl, thiazolyl, furyl, pyranyl, pyrrolyl, 2H-pyrrolyl, imidazolyl, benzymidazolyl, pyrazolyl, isothiazolyl, isoxazolyl and indolyl groups. Preferred nonaromatic heterocyclic groups include in particular the morpholino, piperidinyl, piperazinyl and pyrrolidinyl groups.

The aryl and heterocycle groups may possibly be substituted by an alkyl, alkenyl or alkynyl group. An aryl or a heterocycle substituted by an alkyl group is called an alkylaryl or alkylheterocycle group. Examples of alkylaryl groups include in particular tolyl, mesythyl and xylyl. An aryl or a heterocycle substituted by an alkenyl group is referred to as an alkenylaryl or alkenylheterocycle group. Examples of alkenylaryl groups include in particular the cinnamyl group. An aryl or a heterocycle substituted by an alkynyl group is called an alkynylaryl or alkynylheterocycle group.

The aryl and heterocycle groups may also be substituted by a group independently selected from aryl or heterocycle groups, themselves optionally substituted by one or more substituents preferably selected in the group consisting of a halogen atom and a NO₂, CN, CF₃, OR′, COR′, COOR′, alkoxy, NHCOR′ or CONR′R″ group, R′ and R″ being such as defined hereinabove.

Specific examples of aryl and heterocycle groups substituted by an aryl or heterocycle group are the benzothienyl, benzofuryl, furylphenyl, benzyloxynaphthyl, pyridylphenyl, phenylphenyl and thienylphenyl groups. As noted, the hereinabove groups may be substituted. In this respect one example is the phenyl groups substituted by a phenyl group itself substituted by a halogen atom, a NO₂, CF₃, methoxy or methyl group.

“Alkenyl” groups are linear or branched hydrocarbon functions containing one or more double bonds. Advantageously they contain from 2 to 6 carbon atoms and, preferably, 1 or 2 double bonds. Alkenyl groups may be substituted by an aryl group such as defined hereinabove, in which case it is called an arylalkenyl group.

“Alkynyl” groups are linear or branched hydrocarbon functions containing one or more triple bonds. Advantageously they contain from 2 to 6 carbon atoms and, preferably, 1 or 2 double bonds. Alkynyl groups may be substituted by an aryl group such as defined hereinabove, in which case it is called an arylalkynyl group.

“Alkoxy” groups correspond to the alkyl and cycloalkyl groups defined hereinabove linked to the nucleus by an —O— (ether) bond. Methoxy and ethoxy groups are especially preferred.

“Halogen” designates a fluorine, chlorine, bromine or iodine atom.

“Heteroatom” is an atom selected from O, N and S.

More particularly, the invention has as its object compounds represented by general formula (I) hereinabove in which R₅ is a phenyl group substituted at least in position 3 such as defined hereinabove. Said compounds possess inhibitory properties that are especially marked and preferential for phosphodiesterase 2.

The substituent groups may be selected, for example, in the group consisting of: CHO, CN, CONH2, NO2, CF3, NH2, halogen atom (C1), (C1-C6) alkyl, phenyl optionally substituted, in particular by an acetyl group, by a halogen atom (C1), by a CONH2 group or by a CN, prop-1-ynyl optionally substituted, in particular by a benzyloxy or tert-butyl carbamate group, hex-1-ynyl optionally substituted, in particular by a CN or NH2 group, pentyl optionally substituted, in particular by a CONH2, hexyl, piperidinyl group optionally substituted, in particular by a prop-1-ynyl, benzylaminomethyl, acetamide (CH3CONH), aminomethyl, NH2CS—, 4-phenyl-1, 3-thiazol-2-yl, —CONHBenzyl, —COOEthyl, —CONHiPropyl, —CONH—(CH2)n—CONH2 group (n representing a whole number from 1 to 6), —CONR′R″, with R′ and R″, which are the same or different, representing a C1 -C6 alkyl group or a hydrogen atom, -(4-benzylpyperazin-1-yl)carbonyl, —CONH—(CH2)n-phenyl (n representing a whole number from 1 to 6), imidazolyl, piperazinyl optionally substituted, in particular by a phenyl group.

Among compounds represented by formula (I) wherein R5 is a phenyl group substituted at least in position 3, one can also cite compounds represented by formula (I) in which R5 is a phenyl group substituted in positions 3 and 4, in particular by a hydrocarbon chain possibly containing at least one heteroatom, like oxygen, such as the methylenedioxy (—O—CH2—O—) chain forming a ring with the phenyl group to which it is attached.

Another particular object of the invention is compounds represented by general formula (I) hereinabove in which R5 is the 3-pyridyl, 4-isoquinolyl, piperazinyl group optionally substituted, in particular in position 4 by an aryl group, such as phenyl.

Another particular object of the invention is compounds represented by general formula (I) hereinabove in which Z represents a sulfur atom or —NR2, preferably with R2 forming a ring of the imidazole type with R1.

Particular compounds according to the invention are those in which:
Z is the oxygen atom and/or
R₇ and R₈, independently of each other, represent a OR₁₀ group in which R₁₀ is a (C₁-C₆) alkyl group, preferably an ethyl or methyl group, advantageously methyl, and/or
R₇ and R₈ both represent an ethoxy or methoxy group, advantageously methoxy, or one represents a hydrogen atom and the other an ethoxy or methoxy group, advantageously methyl, and/or
R₆ and R₉, which are the same or different, represent the hydrogen atom, a halogen atom, a phenyl group, a (C₁-C₆) alkyl group or a OR₁₀ group in which R₁₀ is a (C₁-C₆) alkyl group, preferably an ethyl or methyl group, and/or
R₃ and R₃′, which are the same or different, represent a hydrogen atom, and/or
R₁ is a (C₁-C₆) alkyl, (C₆-C₁₈) aryl, such as phenyl, (C₆-C₁₈)aryl(C₁-C₄)alkyl, such as benzyl optionally substituted, or a (C₁-C₁₂)alkyl(C₆-C₁₈)aryl group.

A particular family of compounds is represented by compounds having general formula (I) such as defined hereinabove in which R₃ and R₃′ represent the hydrogen atom.

Another family comprises compounds having general formula (I) in which Z is the oxygen atom, R₇ and R₈, independently of each other, represent a OR₂ group in which R₂ is a (C₁-C₆) alkyl group, R₁ represents the hydrogen atom or a (C₁-C₆) alkyl group, R₆ and R₉ represent the hydrogen atom and R₃ and R_{3′} represent the hydrogen atom.

Another family comprises compounds having general formula (I) in which Z is the oxygen atom, R₇ and R₈, independently of each other, represent a OR₂ group in which R₂ is a (C₁-C₆) alkyl group, R₆ and R₉, which are the-same or different, represent the hydrogen atom, a halogen atom or a (C₁-C₆) alkyl group and R1 represents a (C₁-C₁₂) alkyl, aryl or (C₆-C₁₈)aryl(C₁-C₄)alkyl group, optionally substituted by one or more substituents, which are the same or different, selected in the group consisting of a halogen atom, an alkyl, CF₃, (C₁-C₆) alkoxy group.

According to a particular aspect of the invention, the compounds represented by general formula (I) hereinabove are those in which at least one of the groups R7 and R8, advantageously both, represents a OR₁₀ group where R₁₀ represents a (C₁-C₆) alkyl or (C₃-C₆) cycloalkyl group. Preferably, in the compounds represented by general formula (I) according to the invention and in the particular families mentioned hereinabove, at least one of the groups R₇ and R₈ represents, independently of each another, a methoxy or ethoxy group, advantageously methoxy, more preferably, they both represent a methoxy or ethoxy group, advantageously methoxy.

Preferably, in the compounds represented by general formula (I) according to the invention and in the particular families mentioned hereinabove, the groups R₃ and R_{3′}, which are the same or different, represent a hydrogen atom or a methyl, ethyl or n-propyl group. According to a particularly advantageous variant, in the compounds represented by general formula (I) according to the invention and in the particular families mentioned hereinabove, the groups R₃ and R_{3′} represent a hydrogen atom.

As indicated, in the compounds represented by general formula (I) according to the invention and in the particular families mentioned hereinabove, R1 advantageously represents a hydrogen atom or a (C₁-C₃) alkyl, (C₆-C₁₈) aryl (for example: phenyl), (C₆-C₁₈)aryl(C₁-C₄)alkyl (for example: benzyl), (C₁-C₁₂)alkyl(C₆-C₁₈)aryl group, said group optionally being substituted.

As indicated, in the compounds represented by general formula (I) according to the invention and in the particular families mentioned hereinabove, R₅ is advantageously a phenyl group substituted at least in position 3.

According to a first variant of the invention, R₅ is a phenyl group substituted by:
(a) one or more OR′ groups, in particular methoxy or ethoxy, or
(b) a COR′ group, in particular acetyl or aldehyde, or
(c) a CONR′R″ group, in particular CONH2, or
(d) a CN group, or
(e) a trifluoromethyl group, or
(f) an alkyl group, for example methyl, or alkynyl group, for example hexynyl or propynyl, or
(g) an aryl group or heterocycle, in particular a phenyl, furyl, pyridyl, piperidine, thiazole or thienyl group, said aryl or heterocycle itself optionally being substituted by one or more groups preferably selected from groups (a)-(g).

Especially preferred compounds are selected from the following compounds:
3-(7,8-dimethoxy-2-oxo-2,3-dihydro-1H-1,4-benzodiazepin-5-yl)benzonitrile, 3a
7,8-dimethoxy-[5-(3-trifluoromethyl)phenyl]-1,3-dihydro-2H- 1,4-benzodiazepin-2-one, 3d
3-(7,8-dimethoxy-1-methyl-2-oxo-2,3-dihydro-1H-1,4-benzodiazepin-5-yl)-benzonitrile, 4a
3-[1-(4-chlorobenzyl)-7,8-dimethoxy-2-oxo-2,3 -dihydro-1H-1,4-benzodiazepin-5-yl]-benzonitrile, 4c
3-[1-(3,4-chlorobenzyl)-7,8-dimethoxy-2-oxo-2,3 -dihydro-1H-1,4-benzodiazepin-5-yl]-benzonitrile, 4d
3 -[7,8-dimethoxy-1-(4-methoxybenzyl)-2-oxo-2,3 -dihydro-1H-1,4-benzodiazepin-5-yl]-benzonitrile, 4e
3-[1-(3-chlorobenzyl)-7,8-dimethoxy-2-oxo-2,3-dihydro-1H-1,4-benzodiazepin-5-yl]-benzonitrile, 4f
3-{7,8-dimethoxy-2-oxo-1-[3-(trifluoromethyl)benzyl]-2,3-dihydro-1H-1,4-benzodiazepin-5-yl]-benzonitrile, 4g
3-[1-(2-chlorobenzyl)-7,8-dimethoxy-2-oxo-2,3-dihydro-1H-1,4-benzodiazepin-5-yl]-benzonitrile, 4h
3-{7,8-dimethoxy-2-oxo-1-[4-(trifluoromethyl)benzyl]-2,3-dihydro-1H-1,4-benzodiazepin-5-yl]-benzonitrile, 4i
3-[7,8-dimethoxy-2-oxo-1-(2-phenylethyl)-2,3-dihydro-1H-1,4-benzodiazepin-5-yl]-benzonitrile, 4j
3-(1-ethyl-7,8-dimethoxy-2-oxo-2,3-dihydro-1H-1,4-benzodiazepin-5-yl)benzonitrile, 4k
3-(7,8-dimethoxy-1-propyl-2-oxo-2,3-dihydro-1H-1,4-benzodiazepin-5-yl)benzonitrile, 4l
3-(1-benzyl-7,8-dimethoxy-2-oxo-2,3-dihydro-1H-1,4-benzodiazepin-5-yl)benzonitrile, 4m
ethyl[5-(3-cyanophenyl)-7,8-dimethoxy-2-oxo-2,3-dihydro-1H-1,4-benzodiazepin-1-yl]acetate, 4n
7,8-dimethoxy-1-methyl-[5-(3-trifluoromethyl)phenyl]-1,3-dihydro-2H-1,4-benzodiazepin-2-one, 4p
7,8-dimethoxy-1-ethyl-5-[3-(trifluoromethyl)phenyl]-1,3-dihydro-2H-1,4-benzodiazepin-2-one, 4q
5-[3-(trifluoromethyl)phenyl]-7,8-dimethoxy-1-n-propyl-1,3-dihydro-1,4-benzodiazepin-2-one, 4r
1-benzyl-5-[3-(trifluoromethyl)phenyl]-7,8-dimethoxy-1,3 -dihydro-1,4-benzodiazepin-2-one, 4s
3-(7,8-dimethoxy-1-methyl-2-oxo-2,3-dihydro-1H-1,4-benzodiazepin-5-yl)-benzamide, 5a
3 -(6-bromo-7,8-dimethoxy-1-methyl-2-oxo-2,3-dihydro-1H-1,4-benzodiazepin-5-yl)benzamide, 5b
3-(7,8-dimethoxy-1-methyl-2-oxo-6-phenyl-2,3-dihydro-1H-1,4-benzodiazepin-5-yl)benzamide, 5c
3-(9-bromo-7,8-dimethoxy-1-methyl-2-oxo-2,3-dihydro-1H-1,4-benzodiazepin-5-yl)benzamide, 5d
3-(7,8-dimethoxy-2-oxo-2,3-dihydro-1H-1,4-benzodiazepin-5-yl)benzamide, 5e
3-(7,8-dimethoxy-1-propyl-2-oxo-2,3-dihydro-1H-1,4-benzodiazepin-5-yl)benzamide, 5f
3-(1-ethyl-7,8-dimethoxy-2-oxo-2,3-dihydro-1H-1,4-benzodiazepin-5-yl)benzamide, 5g
3-(1-benzyl-7,8-dimethoxy-2-oxo-2,3-dihydro-1H-1,4-benzodiazepin-5-yl)benzamide, 5h
ethyl{5-[3-(aminocarbonyl)phenyl]-7,8-dimethoxy-2-oxo-2,3-dihydro-1H-1,4-benzodiazepin-1-yl } acetate, 5i
3-(7,8-dimethoxy-1,3-dimethyl-2-oxo-2,3 -dihydro-1H-1,4-benzodiazepin-5-yl) benzamide, 5j
3-[3-(3,4-dichlorobenzyl)-7,8-dimethoxy-1-methyl-2-oxo-2,3-dihydro-1H-1,4-benzodiazepin-5-yl]benzamide, 5k
3-(8-methoxy-1-methyl-2-oxo-2,3-dihydro-1H-1,4-benzodiazepin-5-yl)benzamide, 5l
3-(7,8-dimethoxy-1-methyl-2-oxo-9-phenyl-2,3-dihydro-1H-1,4-benzodiazepin-5-yl)benzamide, 5m
3-(6,8-dimethoxy-2-oxo-2,3-dihydro-1H-1,4-benzodiazepin-5-yl)benzamide, 5n
3-(6,8-dimethoxy-1-methyl-2-oxo-2,3-dihydro-1H-1,4-benzodiazepin-5-yl)benzamide, 5o
tert-butyl-3-[3-(7,8-dimethoxy-1-methyl-2-oxo-2,3-dihydro-1H-1,4-benzodiazepin-5-yl)phenyl]propynylcarbamate, 6a
7,8-dimethoxy-5-(3′-hex-1-ynylphenyl)-1-N-methyl-1,3-dihydro-2H-1,4-benzodiazepin-2-one, 6b
7,8-dimethoxy-1-methyl-5-[3-(3-piperidin-1-ylprop-1-ynyl)phenyl]-1,3-dihydro-2H-1,4-benzodiazepin-2-one, 6c
6-[3-(7,8-dimethoxy-1-methyl-2-oxo-2,3-dihydro-1H-1,4-benzodiazepin-5-yl)phenyl]hex-5-ynenitrile, 6d
7,8-dimethoxy-5-(3′-hexylphenyl)-1-N-methyl-1,3-dihydro-2H-1,4-benzodiazepin-2-one, 6e
5-[3-(3-aminopropyl)phenyl-7,8-dimethoxy-1-methyl-1,3-dihydro-2H-1,4-benzodiazepin-2-one trifluoroacetate, 6h
6-[3-(7,8-dimethoxy-1-methyl-2-oxo-2,3-dihydro-1H-1,4-benzodiazepin-5-yl)phenyl]hexanamide, 6i
5-(4′-chloro-1,1′-biphenyl-3-yl)-7,8-dimethoxy-1-methyl-1,3-dihydro-2H-1,4-benzodiazepin-2-one, 6j
5-{3-[3-(benzyloxy)prop-1-ynyl]phenyl}-1-ethyl-7,8-dimethoxy-1,3-dihydro-2H-1,4-benzodiazepin-2-one, 6k
3′-(7,8-dimethoxy-1-methyl-2-oxo-2,3-dihydro-1H-1,4-benzodiazepin-5-yl)-1,1′-biphenyl-3-carbonitrile, 6l
3′-(7,8-dimethoxy-1-methyl-2-oxo-2,3-dihydro-1H-1,4-benzodiazepin-5-yl)-1,1′-biphenyl-4-carbonitrile, 6m
3′-(7,8-dimethoxy-1-methyl-2-oxo-2,3-dihydro-1H-1,4-benzodiazepin-5-yl)-1,1′-biphenyl-4-carboxamide, 6n
3′-(7,8-dimethoxy-1-methyl-2-oxo-2,3-dihydro-1H-1,4-benzodiazepin-5-yl)-1,1′-biphenyl-3-carboxamide, 6o
3-[3-(3,4-dichlorobenzyl)-7,8-dimethoxy-1-methyl-2-oxo-2,3-dihydro-1H-1,4-benzodiazepin-5-yl]benzonitrile, 7b
7,8-dimethoxy-1,3-dimethyl-5-(3-trifluoromethylphenyl)-1,3-dihydro-2H-1,4-benzodiazepin-2-one, 7c
3-(7,8-dimethoxy-1,3-dimethyl-2-oxo-2,3-dihydro-1H-1,4-benzodiazepin-5-yl)benzonitrile, 7d
5-[3-(aminomethyl)phenyl]-7,8-dimethoxy-1-methyl-1,3-dihydro-2H-1,4-benzodiazepin-2-one, 8a
N-[3-(7,8-dimethoxy-1-methyl-2-oxo-2,3-dihydro-1H-1,4-benzodiazepin-5-yl)benzyl]acetamide, 8b
3-(7,8-dimethoxy-1-methyl-2-oxo-2,3-dihydro-1H-1,4-benzodiazepin-5-yl)thiobenzamide, 9a
7,8-dimethoxy-1-methyl-5-[3-(4-phenyl-1,3-thiazol-2-yl)phenyl]-1,3-dihydro-2H-1,4-benzodiazepin-2-one, 9b
5-(3-cyanophenyl)-7,8-dimethoxy-1,3-dihydro-2H-1,4-benzodiazepin-2-thione, 10d
3-(8,9-dimethoxy-4H-imidazo[1,2-a][1,4]benzodiazepin-6-yl)benzonitrile, 11a
3-(8,9-dimethoxy-4H-imidazo[1,2-a][1,4]benzodiazepin-6-yl)benzamide, 11 b
3-(7,8-dimethoxy-2-methylamino-1,3-dihydro-3H-1,4-benzodiazepin-5-yl)benzonitrile, 12a
7,8-dimethoxy-1-methyl-5-(3-pyridyl)-1,3-dihydro-1,4-benzodiazepin-2-one, 17b
7,8-dimethoxy-1-methyl-5-(3-nitrophenyl)-1,3-dihydro-1,4-benzodiazepin-2-one, 17c
5-(7,8-dimethoxy-1-methyl-2-oxo-2,3-dihydro-1H-1,4-benzodiazepin-5-yl)-2-benzonitrile, 17d
5-(3-acetylphenyl)-7,8-dimethoxy-1-methyl-1,3-dihydro-1,4-benzodiazepin-2-one, 17e
5-(4-isoquinolinyl)-7,8-dimethoxy-1-methyl-1,3-dihydro-1,4-benzodiazepin-2-one, 17f
7,8-dimethoxy-5-(3-hydroxymethylphenyl)-1-methyl-3-propyl-1,3-dihydro-2H-1,4-benzodiazepin-2-one, 17h
5-(3-aminophenyl)-7,8-dimethoxy-1-methyl-1,3-dihydro-2H-1,4-benzodiazepin-2-one,17i
5-(3,4-dichlorophenyl)-7,8-dimethoxy-1-methyl-1,3-dihydro-1,4-benzodiazepin-2-one,17j
7,8-dimethoxy-1-methyl-5-(3-methylphenyl)-1,3-dihydro-1,4-benzodiazepin-2-one, 17k.
5-(3-formylphenyl)-7,8-dimethoxy-1-methyl-1,3-dihydro-1,4-benzodiazepin-2-one, 17l
5-[3-(benzylaminomethyl]phenyl)-7,8-dimethoxy-1-methyl-1,3-dihydro-2H-1,4-benzodiazepin-2-one hydrochloride, 17m
N-[3-(7,8-dimethoxy-1-methyl-2-oxo-2,3-dihydro-1H-1,4-benzodiazepin-5-yl) phenyl]acetamide, 17n
7,8-dimethoxy-1-methyl-5-(3,4-methylenedioxyphenyl)-1,3-dihydro-2H-1,4-benzodiazepin-2-one, 17o
3-(7-hydroxy-8-methoxy-2-oxo-2,3-dihydro-1H-1,4-benzodiazepin-5-yl)benzonitrile, 22b
3-(6-bromo-7-hydroxy-8-methoxy-2-oxo-2,3-dihydro-1H-1,4-benzodiazepin-5-yl)benzonitrile, 23b
3-(9-bromo-8-hydroxy-7-methoxy-2-oxo-2,3-dihydro-1H-1,4-benzodiazepin-5-yl)benzonitrile, 23d
3-(6-bromo-7,8-dimethoxy-1-methyl-2-oxo-2,3-dihydro-1H-1,4-benzodiazepin-5-yl)benzonitrile, 24b
3-(7,8-dimethoxy-1-methyl-2-oxo-6-phenyl-2,3-dihydro-1H-1,4-benzodiazepin-5-yl)benzonitrile, 25b
3-(7,8-dimethoxy-1-methyl-2-oxo-9-phenyl-2,3-dihydro-1H-1,4-benzodiazepin-5-yl)benzonitrile, 25a
tert-butyl-3-[5-(cyanophenyl)-(7,8-dimethoxy-1-methyl-2-oxo-2,3-dihydro-1H-1,4-benzodiazepin-9-yl)phenyl]prop-2-ynylcarbamate,25c
Methyl(2E)-3-[5-(cyanophenyl)-7,8-dimethoxy-1-methyl-2-oxo-2,3-dihydro-1H-1,4-benzodiazepin-9-yl)phenyl]acrulate, 25d
tert-butyl-3-[5-(cyanophenyl)-(7,8-dimethoxy-1-methyl-2-oxo-2,3-dihydro-1H-1,4-benzodiazepin-6-yl)phenyl]prop-2-ynylcarbamate, 25e
[9-(3-aminoethynyl)-7,8-dimethoxy-1-methyl-2-oxo-2,3-dihydro-1H-1,4-benzodiazepin-5-yl]benzonitrile, 25f
[6-(3-aminoethynyl)-7,8-dimethoxy-1-methyl-2-oxo-2,3-dihydro-1-1,4-benzodiazepin-5-yl]benzonitrile, 25g
3-(8-methoxy-2-oxo-2,3-dihydro-1H-1,4-benzodiazepin-5-yl)benzonitrile, 28a
3-(6-methoxy-2-oxo-2,3-dihydro-1H-1,4-benzodiazepin-5-yl)benzonitrile, 28b
3-(7-methoxy-2-oxo-2,3-dihydro-1H-1,4-benzodiazepin-5-yl)benzonitrile, 28c
6-methoxy-5-phenyl-1,3-dihydro-2H-1,4-benzodiazepin-2-one, 28d
7-methoxy-5-phenyl-1,3-dihydro-2H-1,4-benzodiazepin-2-one, 28e
9-bromo-7,8-dimethoxy-5-phenyl-1,3-dihydro-2H-1,4-benzodiazepin-2-one, 28f
3-(6,8-dimethoxy-2-oxo-2,3-dihydro-1H-1,4-benzodiazepin-5-yl)benzonitrile, 28g
3-(7-bromo-6,8-dimethoxy-2-oxo-2,3-dihydro-1H-1,4-benzodiazepin-5-yl)benzonitrile, 28h
3-(8-methoxy-1-methyl-2-oxo-2,3-dihydro-1H-1,4-benzodiazepin-5-yl)benzonitrile, 29a
3-(6,8-dimethoxy-1-methyl-2-oxo-2,3-dihydro-1H-1,4-benzodiazepin-5-yl)benzonitrile, 29b
3-(7-bromo-6,8-dimethoxy-1-methyl-2-oxo-2,3-dihydro-1H-1,4-benzodiazepin-5-yl)benzonitrile, 29c
3-(7,8-dimethoxy-1-methyl-2-oxo-2,3-dihydro-1H-1,4-benzodiazepin-5-yl)methyl benzoate, 34a
3-(7,8-dimethoxy-1-methyl-2-oxo-2,3-dihydro-1H-1,4-benzodiazepin-5-yl)benzoic acid, 35a
3-(7,8-dimethoxy-1-methyl-2-oxo-2,3-dihydro-1H-1,4-benzodiazepin-5-yl)N-isopropylbenzamide, 36a
N-benzyl-3-(7,8-dimethoxy-1-methyl-2-oxo-2,3-dihydro-1H-1,4-benzodiazepin-5-yl) benzamide, 36b
N-(6-amino-6-oxohexyl)-3-(7,8-dimethoxy-1-methyl-2-oxo-2,3-dihydro-1H-1,4-benzodiazepin-5-yl)benzamide, 36c
3-(7,8-dimethoxy-1-methyl-2-oxo-2,3-dihydro-1H-1,4-benzodiazepin-5-yl)-N,N-dimethylbenzamide, 36d
5-{3-[(4-benzylpyperazin-1-yl)carbonyl]phenyl}7,8-dimethoxy-1-methyl-2-oxo-2,3-dihydro-1H-1,4-benzodiazepin-2-one, 36e
3-(7,8-dimethoxy-1-methyl-2-oxo-2,3-dihydro-1H-1,4-benzodiazepin-5-yl)-N-(3-phenylpropyl)benzamide, 36f

Particularly preferred compounds are selected from the following compounds:
3-(1-benzyl-7,8-dimethoxy-2-oxo-2,3-dihydro-1H-1,4-benzodiazepin-5-yl)benzonitrile, 4m
7,8-dimethoxy-1-methyl-[5-(3-trifluoromethyl)phenyl]-1,3-dihydro-2H-1,4-benzodiazepin-2-one, 4p
3-(7,8-dimethoxy-1-methyl-2-oxo-2,3-dihydro-1H1-1,4-benzodiazepin-5-yl)-benzamide, 5a
3-(6-bromo-7,8-dimethoxy-1-methyl-2-oxo-2,3-dihydro-1H-1,4-benzodiazepin-5-yl)benzamide, 5b
tert-butyl-3-[3-(7,8-dimethoxy-1-methyl-2-oxo-2,3-dihydro-1H-1,4-benzodiazepin-5-yl)phenyl]propynylcarbamate, 6a
7,8-dimethoxy-5-(3′-hex-1-ynylphenyl)-1-N-methyl-1,3-dihydro-2H-1,4-benzodiazepin-2-one, 6b
6-[3-(7,8-dimethoxy-1-methyl-2-oxo-2,3-dihydro-1H-1,4-benzodiazepin-5-yl)phenyl]hex-5-ynenitrile, 6d
7,8-dimethoxy-5-(3′-hexylphenyl)-1-N-methyl-1,3-dihydro-2H-1,4-benzodiazepin-2-one, 6e
5-(4′-chloro-1,1 ′-biphenyl-3-yl)-7,8-dimethoxy-1-methyl-1,3-dihydro-2H-1,4-benzodiazepin-2-one, 6j
3 ′-(7,8-dimethoxy-1-methyl-2-oxo-2,3-dihydro-1H-1,4-benzodiazepin-5-yl)-1,1′-biphenyl-4-carbonitrile, 6m
3′-(7,8-dimethoxy-1-methyl-2-oxo-2,3-dihydro-1H-1,4-benzodiazepin-5-yl)-1,1′-biphenyl-4-carboxamide, 6n
3-(3,4-dichlorobenzyl)-1-ethyl-7,8-dimethoxy-5-phenyl-1,3-dihydro-2H-1,4-benzodiazepin-2-one, 7a
7,8-dimethoxy-1-methyl-5-[3-(4-phenyl-1,3-thiazol-2-yl)phenyl]-1,3-dihydro-2H-1,4-benzodiazepin-2-one, 9b
7,8-dimethoxy-1-methyl-5-(3-pyridyl)-1,3-dihydro-1,4-benzodiazepin-2-one, 17b

The compounds according to the invention may be in the form of salts, particularly acid or base salts, preferably compatible with pharmaceutical use. Among the pharmaceutically acceptable acids, non-limiting examples include hydrochloric, hydrobromic, sulfuric, phosphoric, acetic, trifluoroacetic, lactic, pyruvic, malonic, succinic, glutaric, fumaric, tartaric, maleic, citric, ascorbic, methane or ethane sulfonic, camphoric acids, etc. Among the pharmaceutically acceptable bases, non-limiting examples include sodium hydroxide, potassium hydroxide, triethylamine, tert-butylamine, etc.

The invention also relates to a composition, in particular pharmaceutical, comprising a compound such as defined hereinabove, in particular in association with a pharmaceutically acceptable vehicle or excipient.

The PDE2 inhibitors, the compounds represented by formula (I) or the compositions according to the invention may be administered in different ways and in different forms. For instance, they may be administered systemically, by the oral route, by inhalation or by injection, such as for example by the intravenous, intramuscular, subcutaneous, transdermal, intra-arterial route, etc., the intravenous, intramuscular, subcutaneous, oral and inhalation routes being preferred. For injections, the compounds are generally prepared in the form of liquid suspensions, which can be injected through syringes or by infusion, for instance. In this respect, the compounds are generally dissolved in pharmaceutically compatible saline, physiologic, isotonic, buffered solutions and the like, known to those skilled in the art. For instance, the compositions may contain one or more agents or vehicles selected from among dispersives, solubilizers, stabilizers, preservatives, and the like. Agents or vehicles that may be used in the liquid and/or injectable formulations comprise in particular methylcellulose, hydroxymethylcellulose, carboxymethylcellulose, polysorbate 80, mannitol, gelatin, lactose, vegetable oils, acacia and the like.

The compounds may also be administered in the form of gels, oils, tablets, suppositories, powders, capsules, gelules, aerosols, and the like, possibly by means of pharmaceutical forms or devices allowing extended and/or delayed release. For this type of formulation, an agent such as cellulose, carbonates or starches is advantageously used.

It is understood that the injection rate and/or injected dose may be adapted by those skilled in the art according to the patient, the pathology, the mode of administration, etc. Typically, the compounds are administered at doses ranging from 0.1 μg to 100 mg/kg of body weight, more generally from 0.01 to 10 mg/kg, typically between 0.1 and 10 mg/kg. Furthermore, repeated injections may be given, as the case may be. Also, in the case of chronic treatments, delayed or sustained release systems may be advantageous.

The compounds according to the invention can act in particular on phosphodiesterase type PDE2. Thus, the inventive compounds can be (selective) inhibitors of PDE2, that is to say, they show less inhibitory activity towards the other phosphodiesterases and in particular PDE1, PDE3, PDE4 and PDE5. Some of the inventive compounds exhibit an inhibitory profile specific of PDE2, including with respect to adenosine deaminase, and, as such, also have advantageous therapeutic properties.

The PDE2 inhibitor compounds represented by formula (I) according to the invention are of particular interest in treating pathologies involving the central nervous system, in particular due to a deregulation of the function of a neurotransmitter or a deficiency in the release of a neurotransmitter (eg., dopamine, norepinephrine, acetylcholine, etc.), such as more specifically for the treatment of a pathology selected in the group consisting of depression, schizophrenia, anxiety, bipolar disorder, attention deficit disorders, sleep disorders, OCD—obsessive compulsive disorder, fibromyalgia, Tourette's syndrome, pharmacodependence (drugs, medications, alcohol, etc.), epilepsy, Alzheimer's disease, Parkinson's disease, amyotrophic lateral sclerosis, multiple sclerosis, obesity and Lewy body dementia.

The PDE2 inhibitor compounds of the invention are particularly interesting in the treatment of other disorders involving the peripheral nervous system and peripheral organs in general, in particular pathologies of the type reduced natriuria, acute renal failure, hepatic dysfunction, acute hepatic failure, in particular due to age, and pathologies due to or involving dysfunctions of prolactin release, such as restless legs syndrome, rheumatismal, allergic or auto-inflammatory disorders, such as rheumatoid arthritis, rhinitis and asthma.

A particular object of the invention is therefore based on the use of compounds such as described hereinabove for preparing a medicament for treating chronic or acute disorders of the central or peripheral nervous system, or peripheral use of said compounds as vasoconstrictors.

The invention also concerns the use of the compounds as anxiolytic, anticonvulsant, sedative agents or for the treatment of memory or cognitive impairment, in particular mild cognitive impairment.

The invention further concerns the use of the hereinabove compounds for the treatment of neurodegenerative diseases.

In the spirit of the invention, the term treatment designates a preventive or a curative treatment, which can be used alone or in combination with other agents or treatments. Moreover, it can be a treatment of chronic or acute disorders.

The invention also has as object the use of the hereinabove compounds for the treatment of obesity.

The preferred compounds of the invention advantageously show potent inhibitory activity towards PDE2. The preferred compounds of the invention further display an advantageous selectivity profile, in particular a weak activity on PDE3.

The inventive compounds can be prepared from commercially available products, by using a combination of chemical reactions known to those skilled in the art

### LEGENDS OF FIGURES

FIGS. 1 to 7 depict the synthetic routes of compounds represented by formula (I) according to the invention.

FIG. 1: Synthesis of 1,4 benzodiazepinones and corresponding imidazobenzodiazepines, by Route A (Friedel Crafts reaction using a nitrile and AlCl₃/BCl₃ as Lewis acid), with R₁₀═H, CN, Br, CF₃.

FIG. 2: Synthesis of 1,4 benzodiazepinones by Route B (Friedel Crafts reaction using an acid chloride and SnCl₄ as Lewis acid), with R₁₀? CN.

FIG. 3: Route C, via the iminochloride 16 of the benzodiazepinone.

FIG. 4: Synthesis and other substitutions of benzodiazepinones 4.

FIG. 5: Regioselective halogenation of the benzodiazepinone catechol. Direct halogenation is also possible (in the presence of AcOH, NXS) in position R₉ on a benzophenone of type 2 (diagram 1) which leads after ring formation to a benzodiazepinone of type (23a).

FIG. 6: Derivatives substituted on the benzodiazepinone benzo ring.

FIG. 7: Formation of substituted phenyl meta carboxamide compounds.

FIGS. 8 and 9: Results of the elevated plus maze test carried out with an inventive compound.

FIGS. 10 and 11: Results of the swim test carried out with an inventive compound.

FIGS. 12 and 13: Results of the light/dark test carried out with an inventive compound.

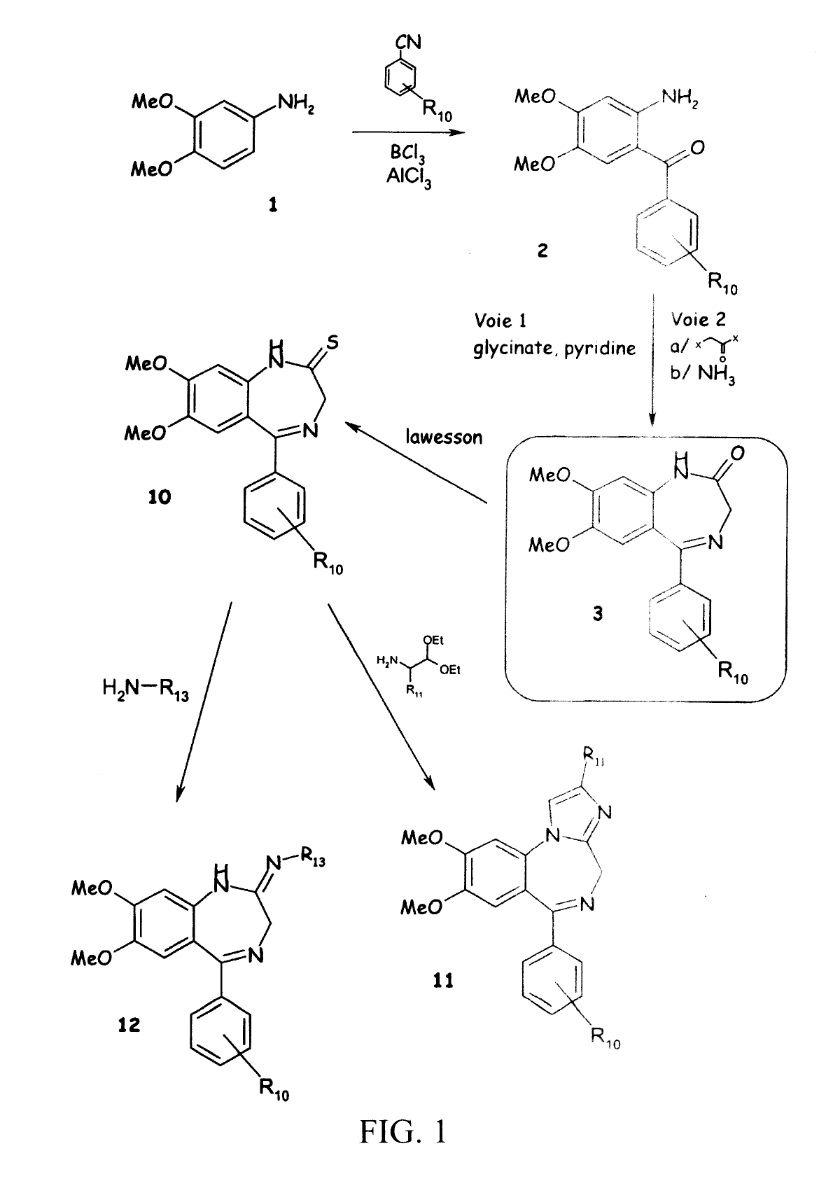

Concerning the methods of preparation of compounds represented by formula (I), and according to a first method shown in FIG. 1, the compounds represented by general formula (I) according to the invention can be obtained by carrying out the following steps starting from a compound represented by general formula I.

Formation of ortho-aminobenzophenones 2:

The Friedel Crafts reaction starting with a compound of general formula 1 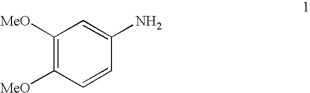
in the presence of a compound of the substituted benzonitrile type, preferably in a halogenated solvent of the type C₂H₄Cl₂, in the presence of a mixture of Lewis acids such as AlCl₃/BCl₃ (Friedel Crafts reaction), followed by hydrolysis of the imine formed in the presence of hydrochloric acid, which leads to a compound of formula 2 in which R₁₀ represents the R5 substituent groups, such as defined hereinabove, or is such as defined in FIG. 1,

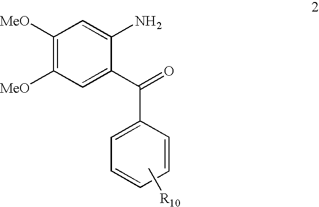

Construction of the benzodiazepinone ring, 3 and derivatives 10-11-12

Route 1 carried out by heating the compound of general formula 2 under reflux in the presence of α-aminoacid ester hydrochloride and pyridine at a temperature comprised between 100° C. and 150° C. leads to formation of a compound having general formula 3. Route 2 carried out by addition of an acetyl halogenide of the type bromoacetyl bromide, followed by ring formation in the presence of ammonia gas in a hydroxylated solvent of the type methanol, leads to the compound having general formula 3 in which R₁₀ is such as defined hereinabove.

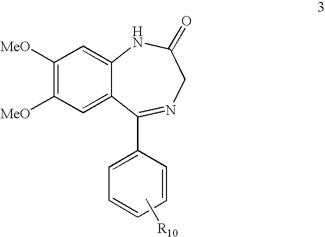

Reaction of a compound of type 3 with the Lawesson reagent in toluene under reflux, can convert a compound represented by formula (I) in which Z is an oxygen atom to a compound represented by formula (I) in which Z is a sulfur atom and thus form a compound of type 10.

Transformation a compound represented by formula (I) in which Z is a sulfur atom to a compound represented by formula (I) in which Z represents NR₁₃ can be carried out in particular by reacting the sulfated compound 10 obtained in the previous step, in the presence of an amine of formula NH₂R₁₃ or by a compound of formula (NH₂)(R₁₁)(CH₂)₂(OEt)₂, R₁₁ and R₁₃ representing a substituent group such as defined hereinabove.

Other substitutions and transformations of benzodiazepinones 3

Reaction in the presence of an alkyl halogenide, preferably in a solvent of the type DMF in the presence of NaH, leads to formation of an N-alkylated compound of general formula 4 in which R₁ and R₁₀ are such as defined hereinabove.

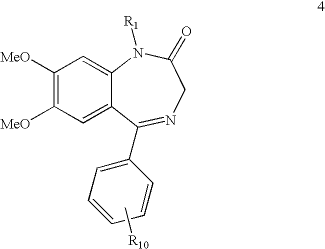

Optionally, transformation of a compound having formula 4 (R₁₀=3—CN) to compound 5 is accomplished by oxidation of the aromatic nitrile function, by reaction with H₂O₂ and NaOH at 50° C. in ethanol.

Optionally, transformation of a compound having formula 4 (R₁₀=3—Br) to comopund 6 is accomplished by palladium coupling in the presence of an aryl boronic acid, or a monosubstituted or monofunctionalized alkyne and a base K₃PO₄, K₂CO₃, triethylamine according to the reaction partners. The Pd(0) or Pd(II) complex is of the type Pd(PPh₃)₄ or PdCl₂, in a solvent of the type DMF, EtOH.

Optionally, transformation of a compound having formula 4 (R₁₀ =3—CN) to compound 8 is accomplished by reduction of the nitrile function by hydrogenation in methanol in the presence of Raney nickel.

Optionally, transformation of a compound having formula 4 to compound 7 is accomplished by alkylation on carbon 3 by reaction of a base, preferably BuLi, in a solvent of the type THF, and addition of an electrophile of the type alkyl, cycloalkyl, benzyl bromide or chloride, substituted or not.

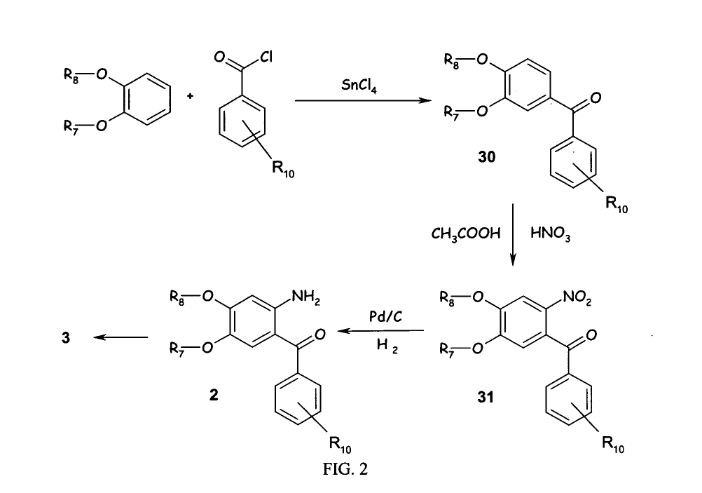

According to a second method illustrated in FIG. 2, compounds represented by general formula (I) can be prepared by a method comprising the following steps:

Reaction of a compound represented by general formula (II) 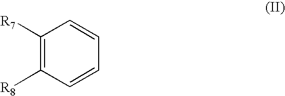
in which R₇ and R₈ are such as defined hereinabove, with an acylating agent, such as a compound of the type benzoyl chloride substituted at least in position 3, in the presence of a Lewis acid, in particular in the presence of SnCl₄, in a halogenated solvent of the type CH₂Cl₂ leads to formation of a benzophenone of formula 30 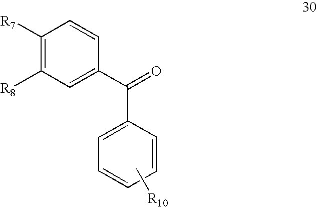
in which R₇ and R₈ are such as defined hereinabove and RIO is a substituent group on the phenyl.

Reaction of the compound of formula 30 in the presence of CH₃COOH and HNO3 at room temperature leads to formation of a nitrated compound of formula 31

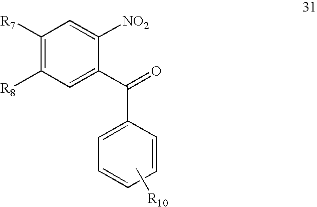

The hydrogenation reaction in the presence of a catalyst of the type Pd/C in methanol gives a compound of type 2

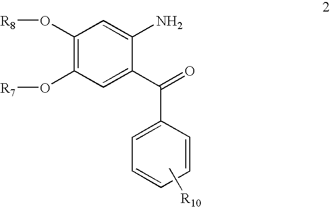

Carrying out route 1 or route 2 starting with a compound of type 2 leads to a compound of type 3.

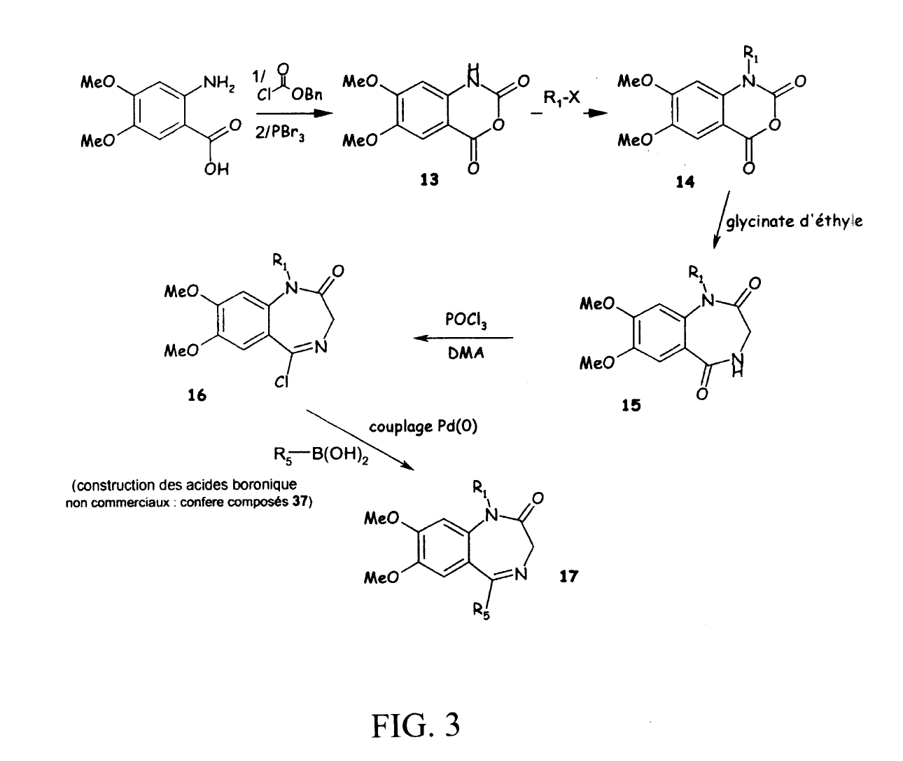

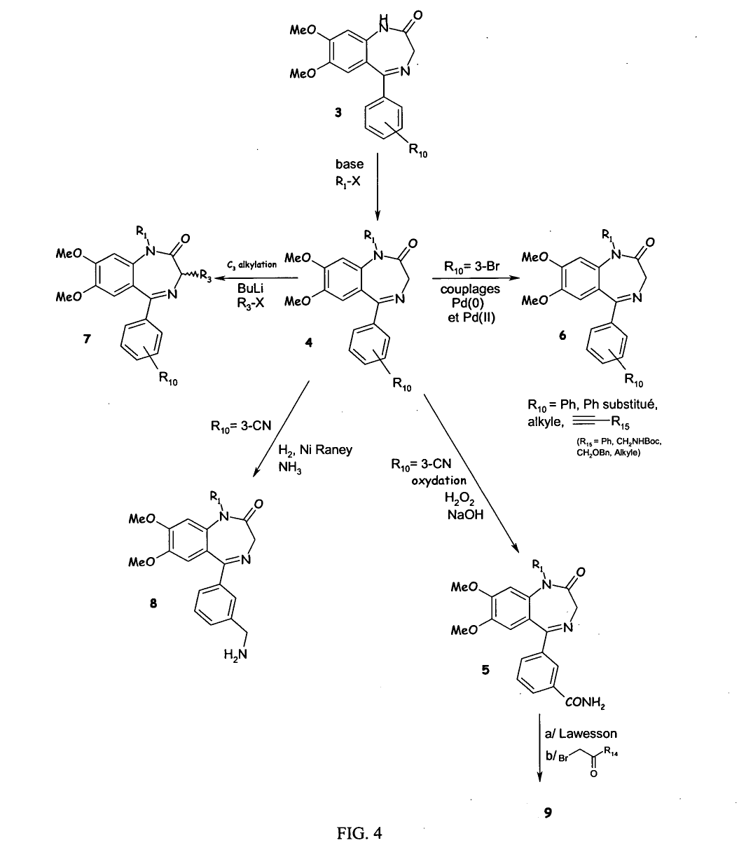

According to another embodiment (FIG. 3), compounds represented by general formula (I) according to the invention in which Z is an oxygen atom can be prepared from a compound represented by general formula 13.

Reaction of a compound of general formula 13: 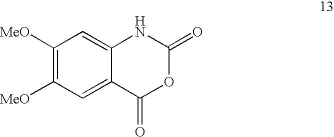
in the presence of an alkyl halogenide, preferably in a solvent of the type DMF in the presence of NaH, leads to an N-alkylated compound of general formula 14 in which R₁ is such as defined hereinabove

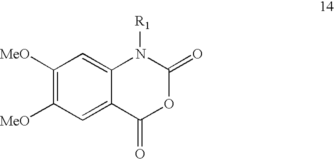

Heating the compound represented by general formula 14 under reflux in the presence of an α-aminoacid ester hydrochloride and pyridine, followed by ring formation in acidic medium, for example in the presence of acetic acid, at a temperature comprised between 100° C. and 150° C., leads to a comopund represented by general formula 15 in which R₁ is such as defined hereinabove.

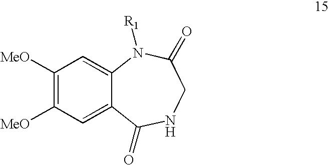

Reaction of the compound represented by general formula 15 in the presence of dimethylaniline (or dimethylaminopyridine) and phosphorus oxyhalogenide (preferably POCl₃), preferably at a temperature comprised between 80° C. and 150° C. in anhydrous CHCl₃ medium and in a sealed tube, leads to the formation of an iminochloride compound represented by general formula 16.

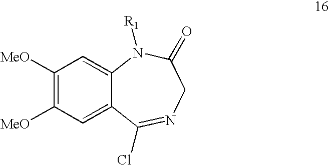

Couplings with a boronic acid of general formula R₅—B(OH)₂ in which R₅ is such as defined hereinabove, in the presence of a base of the type K₃PO₄, K₂CO₃ and a Pd(0) complex of the type Pd(PPh₃)₄, in a solvent of the type DMF, EtOH, leads to the formation of a compound represented by general formula 17.

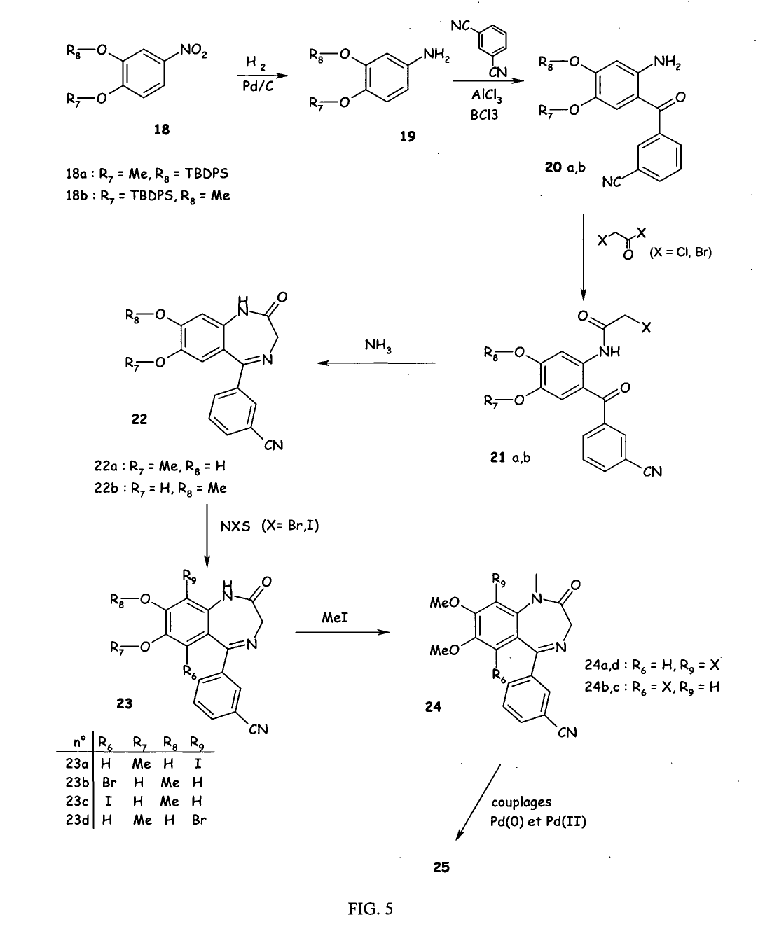

After catalytic hydrogenation of the suitably substituted or protected nitrocatechols 18, compounds 22, which correspond to general formula (I), are prepared according to the routes described earlier, with R₈ and R₇ being defined as in FIG. 5.

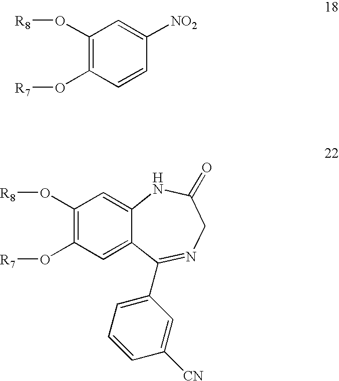

Reaction of a compound 22 in the presence of N-bromo or N-chloro or N-iodo succinimide, in a solvent of the type CH₂Cl₂, and an acid of the type acetic acid leads to a compound represented by general formula 23, with R₈ and R₇ which are defined as in FIG. 5 and in this example R₆ or R₉ represent a halogen atom.

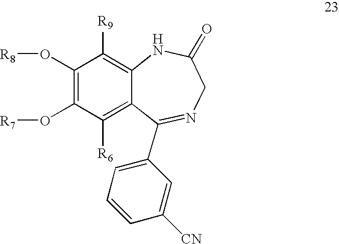

Reaction of compound 23 in the presence of iodomethane, preferably in a solvent of the type DMF in the presence of NaH, leads to the formation of a compound represented by general formula 24 in which R₆ or R₉ represent a halogen atom.

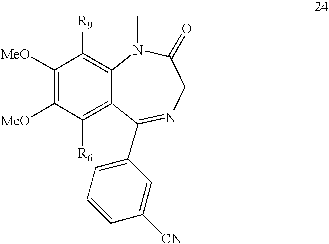

Palladium couplings in the presence of an aryl boronic acid, or a monosubstituted or monofunctionalized alkyne and a base K₃PO₄, K₂CO₃, triethylamine according to the reaction partners. The Pd(0) or Pd(II) complex of the type Pd(PPh₃)₄ or PdCl₂, in a solvent of the type DMF, EtOH leads to the formation of compounds represented by general formula 25.

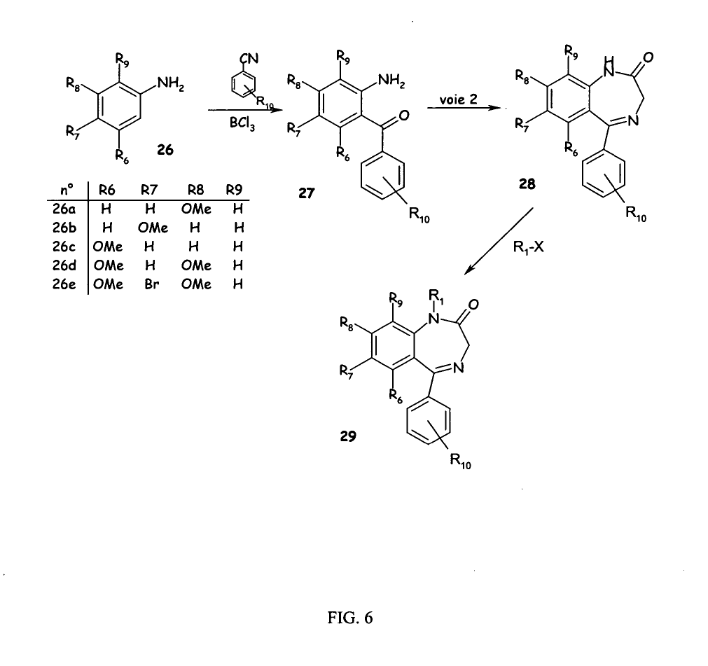

Compounds 29 corresponding to general formula I but with different substitutions or trisubstitutions on the benzodiazepine ring, were prepared according to a method described in FIG. 1 and as illustrated in FIG. 6.

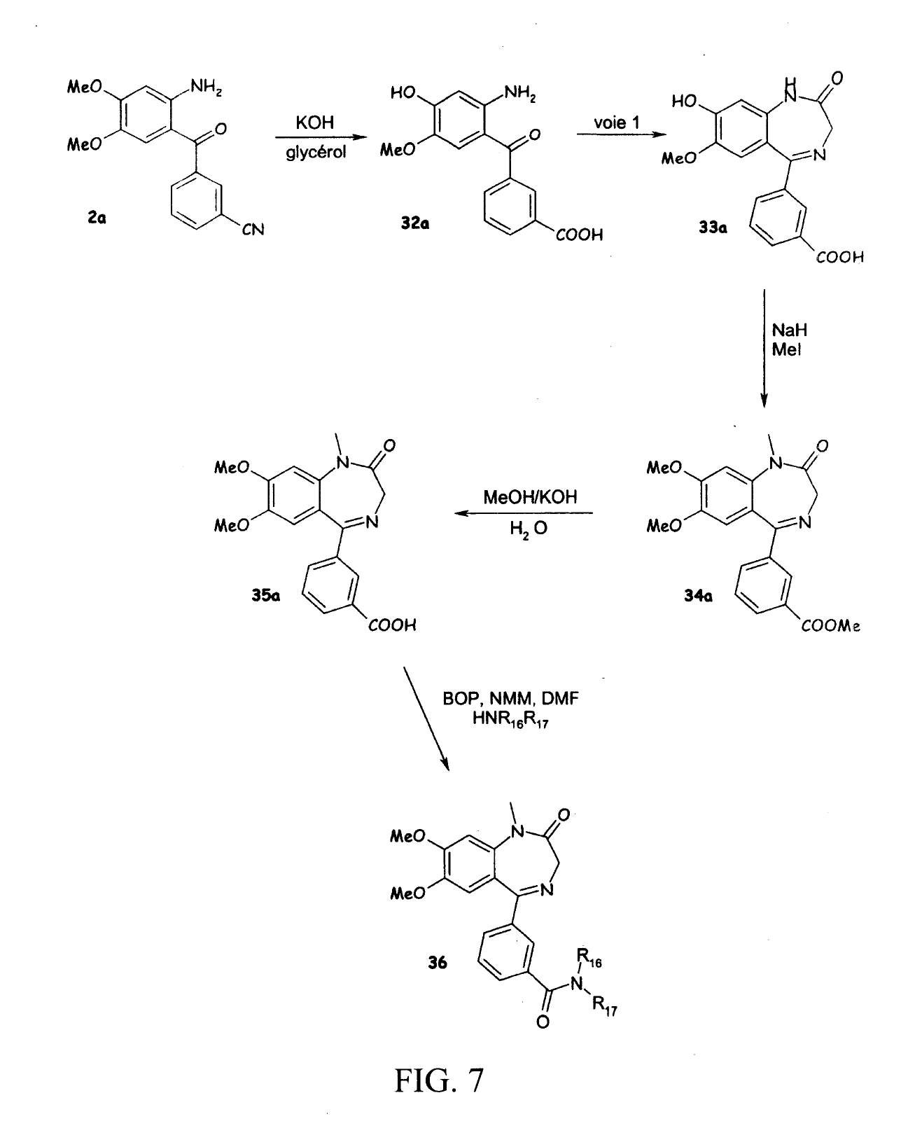

According to another embodiment (FIG. 7), compounds represented by general formula (I) according to the invention in which Z is an oxygen atom can be prepared from a compound 2a

Reaction of the compound 2a:

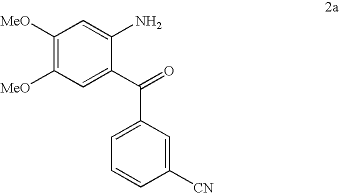

by heating in the presence of a base of the type NaOH, KOH, preferably in a solvent of the alcoholic type such as methanol, ethanol, glycerol, leads to the formation of compound 32a

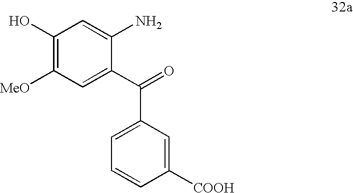

Heating the compound of general formula 32a under reflux in the presence of an α-aminoacid ester hydrochloride and pyridine, at a temperature preferably comprised between 100° C. and 150° C., leads to the formation of a compound 33a

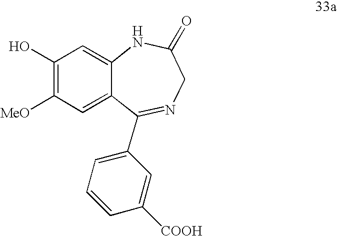

Reaction of a compound 33a in the presence of methyl iodide, preferably in a solvent of the type DMF in the presence of NaH, leads to the formation of a compound represented by general formula 34a

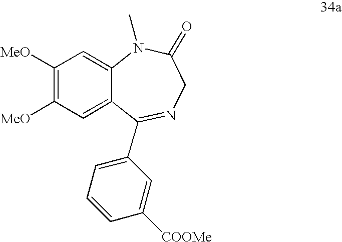

Reaction of the compound 34a by heating in the presence of a base of the type NaOH, KOH, preferably in a solvent of the alcoholic type such as methanol, ethanol, glycerol, leads to the formation of compound 35a

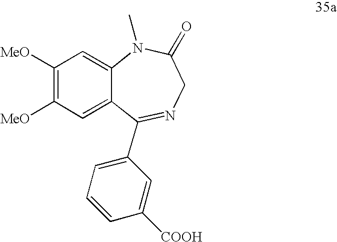

Reaction of compound 35a with a primary or secondary amine, in the presence of a base of the type N-methyl morpholine, BOP in a solvent of the type DMF leads to the formation of amides represented by general formula 36, with R₁₆ and R₁₇ which are defined hereinabove.

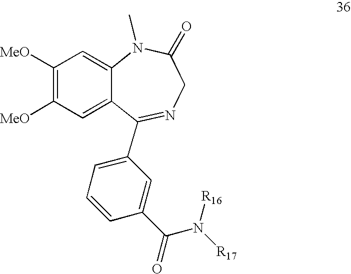

Another object of the invention is based on a method of treatment of a pathology related to a disorder of the central or peripheral nervous system, in particular central, comprising administering to an animal, preferably a human, a PDE2 inhibitor compound, preferably a selective PDE2 inhibitor compound, such as described hereinabove. In particular, the pathologies are those identified hereinabove. The PDE2 inhibitors are preferably 1,4-benzodiazepine derivatives and in particular compounds represented by formula (I).

The invention is illustrated by the following examples, which are given for purposes of illustration and not by way of limitation.

### EXAMPLES

### Example 1

### Synthesis of Compounds Represented by Formula (I)

Synthesis of benzophenones of type 2.

3-(2-amino-4,5-dimethoxybenzoyl)benzonitrile, 2a

At 0° C. under an inert atmosphere, 2.0 g (13.06 mmoles) of 3,4-dimethoxyaniline dissolved in 17 ml of 1,2-dichloroethane, 2.5 g (19.51 mmoles) of isophthalonitrile, and 1.92 g (14.40 mmoles) of AlCl₃ were added to a solution of 14.4 ml of borine tribromide (1M/CH₂Cl₂, 14.4 mmoles). The reaction was stirred at room temperature for 30 minutes, then the dichloromethane was evaporated. The reaction was heated under reflux for 16 hours, then cooled. 14 ml of 1 M HCl at 0° C. were added and the reaction was stirred at 80° C. for 2 hours. After adding 50 ml of water, the reaction was extracted with 3×100 ml of CH₂Cl₂. The organic phases were dried on Na₂SO₄, filtered, evaporated to dryness and purified by chromatography on silica gel (EtOAc/hexane, 1:3). Yield: 61%. ¹H NMR (CDCl₃, 300 MHz): d 3.66 (s, 3H, OCH₃), 3.92 (s, 3H, OCH₃), 6.17-6.48 (m, 3H, NH₂+1H Ar), 6.74 (s, 1H Ar), 7.56-7.91 (m, 4H Ar).

(2-amino-4,5-dimethoxyphenyl)(3-bromophenyl)methanone, 2b

By replacing isophthalonitrile in example 2a by 3-bromobenzonitrile and proceeding in the same manner, the above product was obtained. Yield: 50%. ¹H NMR (CDCl₃, 200 MHz): d 3.67 (s, 3H, OCH₃), 3.92 (s, 3H, OCH₃), 6.20-6.24 (m, 3H, NH₂+1H Ar), 6.86 (s, 1H Ar), 7.29-7.37 (m, 1H Ar), 7.51-7.55 (m, 1H Ar), 7.61-7.65 (m, 1H Ar), 7.75-7.78 (m, 1H Ar).

(2-amino-4,5-dimethoxyphenyl)(phenyl)methanone, 2c

By replacing isophthalonitrile in example 2a by benzonitrile and proceeding in the same manner, the above product was obtained. Yield: 57%. ¹H NMR (CDCl₃, 200 MHz): d 3.66 (s, 3H, OCH₃), 3.91 (s, 3H, OCH₃), 6.21 (m, 3H, NH₂+1H Ar), 6.94 (s, 1H Ar), 7.45-7.64 (m, 5H Ar).

(2-amino-4,5-dimethoxyphenyl)([3-(trifluoromethyl)phenyl]methanone, 2d

By replacing isophthalonitrile in example 2a by 3-(trifluoromethyl)benzonitrile and proceeding in the same manner, the above product was obtained. Yield: 60%. ¹H NMR (CDCl₃, 200 MHz): d 3.66 (s, 3H, OCH₃), 3.93 (s, 3H, OCH₃), 6.21-6.31 (m, 3H, NH₂+1H Ar), 6.74 (s, 1H Ar), 7.57-7.83 (m, 4H Ar).

(2-amino-4,5-diethoxyphenyl)(phenyl)methanone, 2e

By replacing isophthalonitrile in example 2a by benzonitrile, and 3,4-dimethoxyaniline by 3,4-diethoxyaniline and proceeding in the same manner, the above product was obtained. Yield: 35%. ¹H NMR (CDCl₃, 300 MHz): d 1.32 (t, 3H, —CH₃), 1.48 (t, 3H, —CH₃), 3.85 (q, 2H, OCH₂), 4.10 (q, 2H, OCH₂), 6.19 (s, 1H Ar), 6.23 (s, 2H exchangeable, —NH₂), 6.99 (s, 1H Ar), 7.42-7.62 (m, 5H Ar).

Formation of benzodiazepinones of type 3.

3-(7,8-dimethoxy-2-oxo-2,3-dihydro-1H-1,4-benzodiazepin-5-yl)benzonitrile, 3a

To a solution of 3-(2-amino-4,5-dimethoxybenzoyl)benzonitrile 2a (2.0 g, 7.09 mmoles) in dichloromethane (15 ml) at 0-5° C., bromoacetate bromide (0.76 ml, 8.72 mmoles) was added and then 10% Na₂CO₃ (8.5 ml) was added dropwise. The reaction was stirred for 1 hour at this temperature. The two phases were separated, the organic phase was washed with 10 ml of water, dried on Na₂SO₄, filtered, evaporated to dryness (2.8 g). At 0° C. with a CaCl₂ tube, the solid so obtained (2.8 g, 6.94 mmoles) was stirred in NH3 (7N)/MeOH (90 ml) for 3 hours then at room temperature for 1 hour. The mixture was heated under reflux for 3 hours and the precipitate filtered (1.78 g). Yield: 80%. ¹H NMR (CDCl₃, 300 MHz): d 3.75 (s, 3H, OCH₃), 3.98 (s, 3H, OCH₃), 4.50 (broad s, 2H; CH₂), 6.60 (s, 1H Ar), 6.65 (s, 1H Ar), 7.50-7.95 (m, 4H Ar), 9.04 (broad s, 1H, NH).

5-(3-bromophenyl)-7,8-dimethoxy-1,3-dihydro-2H-1,4-benzodiazepin-2-one, 3b

By replacing 3-(2-amino-4,5-dimethoxybenzoyl)benzonitrile 2a in example 3a by (2-amino-4,5-dimethoxyphenyl)(3-bromophenyl)methanone 2b and proceeding in the same manner, the above product was obtained. Yield: 70%. ¹H NMR (CDCl₃, 200 MHz): d 3.75 (s, 3H, OCH₃), 3.97 (s, 3H, OCH₃), 4.32 (broad s, 2H, CH₂), 6.61 (s, 1H, 1H Ar), 6.68 (s, 1H Ar), 7.22-7.30 (m, 1H Ar), 7.46-7.50 (m, 1H Ar), 7.57-7.61 (m, 1H Ar), 7.79-7.80 (m, 1H Ar), 8.83 (s, 1H, NH).

7,8-dimethoxy-5-phenyl-1,3-dihydro-2H-1,4-benzodiazepin-2-one, 3c

By replacing 3-(2-amino-4,5-dimethoxybenzoyl)benzonitrile 2a in example 3a by (2-amino-4,5-dimethoxyphenyl)(phenyl)methanone 2c and proceeding in the same manner, the above product was obtained. Yield: 85%. ¹H NMR (CDCl₃, 200 MHz): d 3.72 (s, 3H, OCH₃), 3.95 (s, 3H, OCH₃), 4.31 (broad s, 2H, CH₂), 6.64 (s, 1H, 1H Ar), 6.70 (s, 1H Ar), 7.37-7.59 (m, 5H Ar), 9.40 (s, 1H, NH).

7,8-dimethoxy-[5-(3-trifluoromethyl)phenyl]-1,3-dihydro-2H-1,4-benzodiazepin-2-one, 3d

By replacing 3-(2-amino-4,5-dimethoxybenzoyl)benzonitrile 2a in example 3a by (2-amino-4,5-dimethoxyphenyl)([3-(trifluoromethyl)phenyl]methanone 2d and proceeding in the same manner, the above product was obtained. Yield: 80%. ¹H NMR (CDCl₃, 300 MHz): d 3.73 (s, 3H, OCH₃), 3.98 (s, 3H, OCH₃), 4.35 (broad s, 2H, CH₂), 6.62 (s, 1H, 1H Ar), 6.67 (s, 1H Ar), 7.50-7.55 (m, 1H Ar), 7.71-7.73 (m, 1H Ar), 7.78-7.81 (m, 1H Ar), 7.91 (m, 1H Ar), 8.67 (s, 1H, NH).

7,8-diethoxy-5-phenyl-1,3-dihydro-2H-1,4-benzodiazepin-2-one, 3e

By replacing 3-(2-amino-4,5-dimethoxybenzoyl)benzonitrile 2a in example 3a by (2-amino-4,5-diethoxyphenyl)(phenyl)methanone 2e and proceeding in the same manner, the above product was obtained. Yield: 60%. MP: 233-236° C. ¹H NMR (CDCl₃, 200 MHz): d 1.39 (t, 3H, CH₃), 1.54 (t, 3H, CH₃), 3.94 (q, 2H, OCH₂), 4.18 (q, 2H, OCH₂), 4.35 (s, 2H, CH₂), 6.66 (s, 1H Ar), 6.74 (s, 1H Ar), 7.36-7.63 (m, 5H Ar), 9.51 (s, 1H exchangeable, —NH).

Alkylation of the nitrogen of type 4.

3-(7,8-dimethoxy-1-methyl-2-oxo-2,3-dihydro-1H-1,4-benzodiazepin-5-yl)-benzonitrile, 4a

Iodomethane (0.42 ml, 6.72 mmoles) was added to a cold mixture of toluene (10 ml) and Aliquat 336 (34 ml). Next, 3-(7,8-dimethoxy-2-oxo-2,3-dihydro-1H-1,4-benzodiazepin-5-yl)-benzonitrile 3a (1.13 g, 3.36 mmoles) and an aqueous solution of 50% sodium hydroxide (4 ml) were added with stirring. The reaction mixture was allowed to return to room temperature and stirred for 4 hours. The reaction was diluted with a 50:50 mixture of dichloromethane/water (200 ml). The phases were separated and the aqueous phase was extracted once with dichloromethane. The organic phases were pooled and dried on sodium sulfate, then filtered, evaporated to dryness and chromatographed. (eluent: CH₂Cl₂/Et₂O, 1:1). A white solid was obtained (1.08 g). Yield: 96%. ¹H NMR (DMSO-d₆, 200 MHz): d 3.41 (s, 3H, NCH₃), 3.76 (s, 3H, OCH₃), 3.99 (s, 3H, OCH₃), 4.30 (AB system, ?d=1.0, J_{AB}=10 Hz, 2H, CH₂), 6.60 (s, 1H Ar), 6.80 (s, 1H Ar), 7.49-7.96 (m, 4H Ar).

5-(3-bromophenyl)-7,8-dimethoxy-1-methyl-1,3-dihydro-2H-1,4-benzodiazepin-2-one, 4b

By replacing 3-(7,8-dimethoxy-2-oxo-2,3-dihydro-1H-1,4-benzodiazepin-5-yl)-benzonitrile 3a in example 4a by 5-(3-bromophenyl)-7,8-dimethoxy-1,3-dihydro-2H-1,4-benzodiazepin-2-one 3b and proceeding in the same manner, the above product was obtained. Yield: 70%. ¹H NMR (CDCl₃, 200 MHz): d 3.40 (s, 3H, NCH₃), 3.77 (s, 3H, OCH₃), 3.99 (s, 3H, OCH₃), 4.30 (AB system, ?d=1.1, J_{AB}=10 Hz, 2H, CH₂), 6.66 (s, 1H Ar), 6.78 (s, 1H Ar), 7.26 (m, 1H Ar), 7.55 (m, 2H Ar), 7.84 (m, 1H Ar).

3-[1-(4-chlorobenzyl)-7,8-dimethoxy-2-oxo-2,3-dihydro-1H-1,4-benzodiazepin-5-yl]-benzonitrile, 4c

By replacing iodomethane in example 4a by 4-chlorobenzyl bromide and proceeding in the same manner, the above product was obtained. Yield: 75%. ¹H NMR (CDC1₃, 300 MHz): d 3.71 (s, 3H, OCH₃), 3.88 (s, 3H, OCH₃), 4.40 (AB system, ?d=1.0, J_{AB}=10 Hz, 2H, CH₂), 5.10 (AB system, ?d=0.8, J_{AB}=15 Hz, 2H, NCH₂), 6.46 (s, 1H Ar), 6.82 (s, 1H Ar), 6.97-7.00 (m, 2H Ar), 7.10-7.13 (m, 2H Ar), 7.46-7.55 (m, 2H Ar), 7.73-7.79 (m, 2H Ar).

3-[1-(3,4-chlorobenzyl)-7,8-dimethoxy-2-oxo-2,3-dihydro-1H-1,4-benzodiazepin-5-yl]-benzonitrile, 4d

By replacing iodomethane in example 4a by 3,4-dichlorobenzyl chloride and proceeding in the same manner, the above product was obtained. Yield: 65%. ¹H NMR (CDCl₃, 300 MHz): d 3.73 (s, 3H, OCH₃), 3.90 (s, 3H, OCH₃), 4.40 (AB system, ?d=1.0, J_{AB}=10 Hz, 2H, CH₂), 5.10 (AB system, ?d=0.8, J_{AB}=15 Hz, 2H, NCH₂), 6.52 (s, 1H Ar), 6.80 (s, 1H Ar), 6.93-6.95 (d, 1H Ar), 7.09 (s, 1H Ar), 7.23-7.26 (d, 1H Ar), 7.51-7.53 (t, 1H Ar), 7.64-7.67 (d, 1H Ar), 7.75-7.77 (d, 1H Ar), 7.86 (s, 1H Ar).

3-[7,8-dimethoxy-l-(4-methoxybenzyl)-2-oxo-2,3-dihydro-1H-1,4-benzodiazepin-5-yl]-benzonitrile, 4e

By replacing iodomethane in example 4a by 4-methoxybenzyl chloride and proceeding in the same manner, the above product was obtained. Yield: 60%. ¹H NMR (CDCl₃, 300 MHz): d 3.70 (s, 3H, OCH₃), 3.74 (s, 3H, OCH₃), 4.40 (AB system, ?d=1.0, J_{AB}=10 Hz, 2H, CH₂), 3.90 (s, 3H, OCH₃), 5.10 (AB system, ?d=0.9, J_{AB}=15 Hz, 2H, NCH₂), 6.42 (s, 1H Ar), 6.65-6.68 (d, 2H Ar), 6.87 (s, 1H Ar), 6.95-6.97 (d, 2H Ar), 7.45-7.50 (t, 1H Ar), 7.62 (s, 1H Ar), 7.61-7.65 (d, 1H Ar), 7.68-7.70 (d, 1H Ar).

3-[1-(3-chlorobenzyl)-7,8-dimethoxy-2-oxo-2,3-dihydro-1H-1,4-benzodiazepin-5-yl]-benzonitrile, 4f

By replacing iodomethane in example 4a by 3-chlorobenzyl bromide and proceeding in the same manner, the above product was obtained. Yield: 70%. ¹H NMR (CDCl₃, 300 MHz): d 3.72 (s, 3H, OCH₃), 3.89 (s, 3H, OCH₃), 4.40 (AB system, ?d=1.0, J_{AB}=10 Hz, 2H, CH₂), 5.10 (AB system, ?d=0.9, J_{AB}=15 Hz, 2H, NCH₂), 6.49 (s, 1H Ar), 6.81 (s, 1H Ar), 6.99-7.09 (m, 2H Ar), 7.11-7.18 (t, 1H Ar), 7.20 (m, 1H Ar), 7.48-7.54 (t, 1H Ar), 7.69-7.74 (m, 3H Ar).

3-{7,8-dimethoxy-2-oxo-1-[3-(trifluoromethyl)benzyll-2,3-dihydro-1H-1,4-benzodiazepin-5-yl]-benzonitrile, 4g

By replacing iodomethane in example 4a by 3-trifluoromethyl)benzyl chloride and proceeding in the same manner, the above product was obtained. Yield: 70%. ¹H NMR (CDCl₃, 300 MHz): d 3.72 (s, 3H, OCH₃), 3.88 (s, 3H, OCH₃), 4.40 (AB system, ?d =1.0, J_{AB}=10 Hz, 2H, CH₂), 5.20 (AB system, ?d=0.8, J_{AB}=15 Hz, 2H, NCH₂), 6.50 (s, 1H Ar), 6.81 (s, 1H Ar), 7.29-7.31 (m, 3H Ar), 7.46-7.52 (m, 2H Ar), 7.69-7.75 (m, 3H Ar).

3-[1-(2-chlorobenzyl)-7,8-dimethoxy-2-oxo-2,3-dihydro-1H-1,4-benzodiazepin-5-y]-benzonitrile, 4h

By replacing iodomethane in example 4a by 2-chlorobenzyl bromide and proceeding in the same manner, the above product was obtained. Yield: 60%. ¹H NMR (CDCl₃, 300 MHz): d 3.70 (s, 3H, OCH₃), 3.92 (s, 3H, OCH₃), 4.40 (AB system, ?d=1.0, J_{AB}=10 Hz, 2H, CH₂), 5.30 (AB system, ?d=0.6, J_{AB}=15 Hz, 2H, NCH₂), 6.45 (s, 1H Ar), 6.88 (s, 1H Ar), 6.95-6.96 (m, 2H Ar), 7.13-7.18 (m, 1H Ar), 7.28-7.30 (m, 1H Ar), 7.48-7.51 (m, 1H Ar), 7.54 (s, 1H Ar), 7.66-7.74 (m, 2H Ar).

3-{7,8-dimethoxy-2-oxo-1-[4-(trifluoromethyl)benzyl]-2,3-dihydro-1H-1,4-benzodiazepin-5-yl}-benzonitrile, 4i

By replacing iodomethane in example 4a by (4-trifluoromethyl)benzyl bromide and proceeding in the same manner, the above product was obtained. Yield: 70%. ¹H NMR (CDCl₃, 300 MHz): d 3.47 (s, 3H, OCH₃), 3.71 (s, 3H, OCH₃), 4.40 (AB system, ?d =1.0, J_{AB}=10 Hz, 2H, CH₂), 5.20 (AB system, ?d=0.8, J_{AB}=15 Hz, 2H, NCH₂), 6.48 (s, 1H Ar), 6.82 (s, 1H Ar), 7.17-7.20 (m, 2H Ar), 7.40-7.47 (m, 4H Ar), 7.73 (m, 1H Ar), 7.87 (m, 1H Ar).

3-[7,8-dimethoxy-2-oxo-l-(2-phenylethyl)-2,3-dihydro-1H-1,4-benzodiazepin-5-yl]-benzonitrile, 4j

By replacing iodomethane in example 4a by benzyl 2-bromoethylbenzene and proceeding in the same manner, the above product was obtained. Yield: 65%. ¹H NMR (CDCl₃, 300 MHz): d 2.88-2.94 (m, 2H, Ph—CH₂), 3.74 (s, 3H, OCH₃), 3.92 (s, 3H, OCH₃), 4.15 (AB system, ?d=0.8, J_{AB}=15 Hz, 2H, NCH₂), 4.40 (AB system, ?d=0.8, J_{AB}=14 Hz, 2H, CH₂), 6.50 (s, 1H Ar), 6.75 (s, 1H Ar), 7.08-7.12 (m, 5H Ar), 7.47-7.50 (t, 1H Ar), 7.53 (s, 1H Ar), 7.67 (d, 1H Ar), 7.72 (d, 1H Ar).

3-(1-ethyl-7,8-dimethoxy-2-oxo-2,3-dihydro-1H-1,4-benzodiazepin-5-yl)benzonitrile, 4k

3-(7,8-dimethoxy-2-oxo-2,3-dihydro-1H-1,4-benzodiazepin-5-yl)-benzonitrile 3a (200 mg, 0.65 mmole) was added to a cold mixture of DMF (4 ml) and 60% NaH (29 mg, 0.71 mmole). The solution was stirred at room temperature for 30 minutes, then iodoethane (67 μL, 0.84 mmole) was added at 0° C. The solution was stirred at room temperature for 1 hour. 30 ml of a water/ice mixture was added and the aqueous phase was extracted with 3×30 ml of Et₂O. The organic phases were pooled, dried on sodium sulfate, filtered, evaporated to dryness and chromatographed on silica gel (eluent: EtOAc). A white solid was obtained (110 mg). Yield: 48%. ¹H NMR (CDCl₃, 200 MHz): d 1.13-1.20 (t, 3H, —CH₃), 3.66-3.78 (m, 1H, NCH₂), 3.80 (s, 3H, OCH₃), 4.02 (s, 3H, OCH₃), 4.29-4.40 (m, 1H, NCH₂), 4.34 (AB system, ?d=1.0, J_{AB}=10 Hz, 2H, CH2), 6.61 (s, 1H Ar), 6.90 (s, 1H Ar), 7.57-7.61 (t, 1H Ar), 7.77-7.80 (d, 1H Ar), 7.93-7.99 (m, 2H Ar).

3-(7,8-dimethoxy-1-propyl-2-oxo-2,3-dihydro-1H-1,4-benzodiazepin-5-yl)benzonitrile, 41 l

By replacing iodoethane in example 4k by iodopropane and proceeding in the same manner, the above product was obtained. Yield: 49%. 1H NMR (CDCl₃, 200 MHz): d 0.75-0.82 (t, 3H, —CH₃), 1.42-1.58 (m, 2H, CH₂—CH₃), 3.48-3.58 (m, 1H, NCH₂), 3.80 (s, 3H, OCH₃), 3.83 (AB system, ?d=1.0, J_{AB}=10 Hz, 2H, CH2), 4.02 (s, 3H, OCH₃), 4.32-4.46 (m, 1H, NCH₂), 4.83 (AB system, ?d=1.0, J_{AB}=10 Hz, 2H, CH2), 6.61 (s, 1H Ar), 6.89 (s, 1H Ar), 7.57-7.61 (t, 1H Ar), 7.77-7.80 (d, 1H Ar), 7.94-8.00 (m, 2H Ar).

3-(1-benzyl-7,8-dimethoxy-2-oxo-2,3-dihydro-1H-1,4-benzodiazepin-5-yl)benzonitrile, 4m

By replacing iodoethane in example 4k by benzyl bromide and proceeding in the same manner, the above product was obtained. Yield: 49%. ¹H NMR (CDCl₃, 200 MHz): d 3.69 (s, 3H, OCH₃), 3.88 (s, 3H, OCH₃), 4.40 (AB system, ?d=1.0, J_{AB}=10 Hz, 2H, CH2), 5.13 (AB system, ?d=0.85, J_{AB}=15 Hz, 2H, CH2), 6.43 (s, 1H Ar), 6.86 (s, 1H Ar), 7.01-7.26 (m, 4H Ar), 7.44-7.55 (m, 2H Ar), 7.66-7.71 (m, 2H Ar).

Ethyl[5-(3-cyanophenyl)-7,8-dimethoxy-2-oxo-2,3-dihydro-1H-1,4-benzodiazepin-1-yl]acetate, 4n

By replacing iodoethane in example 4k by ethyl bromoacetate and proceeding in the same manner, the above product was obtained. Yield: 55%. ¹H NMR (CDCl₃, 200 MHz): d 1.22 (t, 3H, CH₃), 3.75 (s, 3H, OCH₃), 3.94 (s, 3H, OCH₃), 4.18 (m , 2H, OCH₂), 4.36 (AB system, ?d=0.93, J_{AB}=10 Hz, 2H, CH2), 4.51 (s, 2H, NCH₂), 6.58 (s, 1H Ar), 6.83 (s, 1H Ar), 7.49-7.57 (t, 1H Ar), 7.72-7.76 (d, 1H Ar), 7.92-7.97 (m, 2H Ar).

7,8-dimethoxy-1-ethyl-5-phenyl-1,3-dihydro-2H-1,4-benzodiazepin-2-one, 4o

By replacing 3-(7,8-dimethoxy-2-oxo-2,3-dihydro-1H-1,4-benzodiazepin-5-yl)-benzonitrile 3a in example 4a by 7,8-dimethoxy-5-phenyl-1,3-dihydro-2H-1,4-benzodiazepin-2-one 3c and iodomethane by iodoethane, and proceeding in the same manner, the above product was obtained. Yield: 95%. ¹H NMR (CDCl₃, 200 MHz): d 1.11 (t, 3H CH₃), 3.58-3.70 (m, 1H NCH₂), 3.75 (s, 3H, OCH₃), 3.97 (s, 3H, OCH₃), 4.27 (AB system, ?d=0.98, J_{AB}=10 Hz, 2H, CH₂), 4.29-4.39 (m, 1H NCH₂), 6.68 (s, 1H Ar), 6.84 (s, 1H Ar), 7.39-7.45 (m, 3H Ar), 7.62-7.65 (m, 2H Ar).

7,8-dimethoxy-1-methyl-[5-(3-trifuoromethyl)phenyl]-1,3-dihydro-2H-1,4-benzodiazepin-2-one, 4p

By replacing 3-(7,8-dimethoxy-2-oxo-2,3-dihydro-1H-1,4-benzodiazepin-5-yl)-benzonitrile 3a in example 4a by 7,8-dimethoxy-[5-(3-trifluoromethyl)phenyl]-1,3-dihydro-2H-1,4-benzodiazepin-2-one 3d and proceeding in the same manner, the above product was obtained. Yield: 95%. ¹H NMR (CDCl₃, 200 MHz): d 3.42 (s, 3H, NCH₃), 3.81 (s, 3H, OCH₃), 4.00 (s, 3H, OCH₃), 4.30 (AB system, ?d=1.0, J_{AB}=10 Hz, 2H, CH₂), 6.65 (s, 1H Ar), 6.81 (s, 1H Ar), 7.52-7.57 (m, 1H Ar), 7.72-7.74 (m, 1H Ar), 7.88-7.91 (m, 1H Ar), 7.95 (s, 1H Ar).

7,8-dimethoxy-l-ethyl-5-[3-(trifluoromethyl)phenyl]-1,3-dihydro-2H-1,4-benzodiazepin-2-one, 4q

By replacing 3-(7,8-dimethoxy-2-oxo-2,3-dihydro-1H-1,4-benzodiazepin-5-yl)-benzonitrile 3a in example 4a by 7,8-dimethoxy-[5-(3-trifluoromethyl)phenyl]-1,3-dihydro-2H-1,4-benzodiazepin-2-one 3d, and iodomethane by iodoethane, and proceeding in the same manner, the above product was obtained. Yield: 71%. ¹H NMR (CDCl₃, 300MHz): d 1.14 (t, 3H, —CH₃), 3.76 (s, 3H, OCH₃), 3.99 (s, 3H, OCH₃), 4.00 (ABX system, ?d=0.61, J_{AX}=J_{BX}=13.9, 2H, —NCH₂), 4.31 (AB system, ?d=1.01, J_{AB}=10, 2H, CH₂), 6.62 (s, 1H Ar), 6.87 (s, 1H Ar), 7.51-7.57 (t, 1H Ar), 7.71-7.74 (d, 1H Ar), 7.80-7.85 (d, 1H Ar), 7.96 (s, 1H Ar).

5-[3-(trifluoromethyl)phenyl]-7,8-dimethoxy-1-n-propyl-1,3-dihydro-1,4-benzodiazepin-2-one, 4r

By replacing 3-(7,8-dimethoxy-2-oxo-2,3-dihydro-1H-1,4-benzodiazepin-5-yl)benzonitrile 3a in example 4a by 7,8-dimethoxy-5-(3-trifluoromethylphenyl)-1,3-dihydro-1,4-benzodiazepin-2-one 3d and iodomethane by bromopropane, and proceeding in the same manner, the above product was obtained. Yield: 75%. MP: 135-137° C. ¹H NMR (CDCl₃, 200 MHz): d 0.74-0.82 (m, 3H, CH₂CH₃), 1.49-1.63 (m, 2H, CH₂CH₃), 3.49-3.62 (m, 1H, CH), 3.78 (s, 3H, OCH₃), 4.01 (s, 3H, OCH₃), 4.34 (AB system, ?d=1.00, J_{AB}=10.0, 2H, CH₂), 4.31-4.42 (m, 1H, CH), 6.65 (s, 1H Ar), 7.89 (s, 1H Ar), 7.53-7.99 (m, 4H Ar).

1-benzyl-5-[3-(trifluoromethyl)phenyl]-7,8-dimethoxy-1,3-dihydro-1,4-benzodiazepin-2-one, 4s

By replacing 3-(7,8-dimethoxy-2-oxo-2,3-dihydro-1H-1,4-benzodiazepin-5-yl)benzonitrile 3a in example 4a by 7,8-dimethoxy-5-(3-trifluoromethylphenyl)-1,3-dihydro-1,4-benzodiazepin-2-one 3d and iodomethane by benzyl bromide, and proceeding in the same manner, the above product was obtained. Yield: 80%. MP: 175-178° C. ¹H NMR (CDCl₃, 200 MHz): d 3.71 (s, 3H, OCH₃), 3.90-3.98 (m, 4H, OCH₃+CH), 4.92-4.97 (m, 1H, CH), 5.19 (AB system, ?d=0.80, J_{AB}=15, 2H, CH₂), 6.51 (s, 1H Ar), 6.88 (s, 1H Ar), 7.08-7.76 (m, 9H Ar).

Oxidation of the nitrile function of type 5.

3-(7,8-dimethoxy-1-methyl-2-oxo-2,3-dihydro-1H-1,4-benzodiazepin-5-yl)-benzamide, 5a

Aqueous H₂O₂ (30% m/m in water, 7.6 ml) and NaOH (0.5 M, 10 ml) were added dropwise to a solution of 3-(7,8-dimethoxy-1-methyl-2-oxo-2,3-dihydro-1H-1,4-benzodiazepin-5-yl)-benzonitrile 4a (7.5 g, 22.4 mmoles) in ethanol (100 ml). The mixture was stirred at 60° C. for 2 hours then cooled to room temperature. A saturated Na₂S₂0₃ solution (10 ml) was then added and the mixture was stirred for 15 minutes. The ethanol was evaporated, the reaction medium was diluted with water (100 ml) and extracted with EtOAc (3×100 ml). The pooled organic phases were dried on Na₂SO₄, filtered and evaporated to dryness. Recrystallization was in ethanol. A white solid was obtained. Yield: 75%. ¹H NMR (CDCL₃, 300 MHz): □ 0 3.42 (s, 3H, NCH₃), 3.74 (s, 3H, OCH₃), 3.99 (s, 3H, OCH₃), 4.33 (AB system, □□=1.0, J_{AB}=10 Hz, 2H, CH₂), 5.89 (s, 1H, NHH), 6.39 (s, 1H, NHH), 6.65 (s, 1H Ar), 6.80 (s, 1H Ar), 7.47-7.50 (t, 1H Ar), 7.77 (d, 1H Ar), 7.95 (d, 1H Ar), 8.15 (s, 1H Ar).

3-(6-bromo-7,8-dimethoxy-1-methyl-2-oxo-2,3-dihydro-1H-1,4-benzodiazepin-5-yl)benzamide, 5b

By replacing 3-(7,8-dimethoxy-1-methyl-2-oxo-2,3-dihydro-1H-1,4-benzodiazepin-5-yl)benzonitrile 4a in example 5a by 3-(6-bromo-7,8-dimethoxy-1-methyl-2-oxo-2,3-dihydro-1H-1,4-benzodiazepin-5-yl)benzonitrile 24b and proceeding in the same manner, the above product was obtained. Yield: 65%. ¹H NMR (CDCl₃, 200 MHz): d 3.39 (s, 3H, NCH₃), 3.86 (s, 3H, OCH₃), 4.00 (s, 3H, OCH₃), 4.30 (AB. system, ?d =0.9, J_{AB}=10 Hz, 2H, NCH₂), 6.83 (s, 1H Ar), 7.44 (t, 1H Ar), 7.63 (d, 1H Ar), 7.86 (d, 1H Ar), 8.00 (s, 1H Ar).

3-(7,8-dimethoxy-1-methyl-2-oxo-6-phenyl-2,3-dihydro-1H-1,4-benzodiazepin-5-yl)benzamide, 5c

By replacing 3-(7,8-dimethoxy-1-methyl-2-oxo-2,3-dihydro-1H-1,4-benzodiazepin-5-yl)benzonitrile 4a in example 5a by 3-(7,8-dimethoxy-1-methyl-2-oxo-6-phenyl-2,3-dihydro-1H-1,4-benzodiazepin-5-yl)benzonitrile 25b and proceeding in the same manner, the above product was obtained. Yield: 95%. ¹H NMR (DMSO-d₆,300 MHz): d 3.39 (s, 3H, NCH₃), 3.46 (s, 3H, OCH₃), 4.02 (s, 3H, OCH₃), 4.40 (AB system, ?d=0.5, J_{AB}=10 Hz, 2H, NCH₂), 6.82-6.85 (m, 2H Ar), 7.00-7.15 (m, 4H Ar), 7.24-7.26 (m, 2H Ar), 7.45 (s, 1H Ar), 7.62-7.64 (d, 1H Ar), 10.21 (s, 2H, NH₂).

3-(9-bromo-7,8-dimethoxy-1-methyl-2-oxo-2,3-dihydro-1H-1,4-benzodiazepin-5-yl)benzamide, 5d

By replacing 3-(7,8-dimethoxy-1-methyl-2-oxo-2,3-dihydro-1H-1,4-benzodiazepin-5-yl)benzonitrile 4a in example 5a by 3-(9-bromo-7,8-dimethoxy-1-methyl-2-oxo-2,3-dihydro-1H-1,4-benzodiazepin-5-yl)benzonitrile 24d and proceeding in the same manner, the above product was obtained. Yield: 60%. ¹H NMR (DMSO-d₆, 200 MHz): d 3.18 (s, 3H, NCH₃), 3.74 (s, 3H, OCH₃), 3.89 (s, 3H, OCH₃), 4.20 (AB system, ?d=0.8, J_{AB}=11 Hz, 2H, NCH₂), 6.87 (s, 1H Ar), 7.46 (broad s, 1H, NH), 7.54-7.61 (t, 1H Ar), 7.85-7.89 (d, 1H Ar), 8.03-8.20 (m, 3H, 2H Ar and NH).

3-(7,8-dimethoxy-2-oxo-2,3-dihydro-1H-1,4-benzodiazepin-5-yl)benzamide, 5e

By replacing 3-(7,8-dimethoxy-1-methyl-2-oxo-2,3-dihydro-1H-1,4-benzodiazepin-5-yl)benzonitrile 4a in example 5a by 3-(7,8-dimethoxy-2-oxo-2,3-dihydro-1H-1,4-benzodiazepin-5-yl)-benzonitrile 3a and proceeding in the same manner, the above product was obtained. Yield: 67%. ¹H NMR (CDCl₃, 200 MHz): d 3.19 (s, 3H, OCH₃), 3.44 (s, 3H, OCH₃), 3.73 (s, 2H, CH₂), 6.14 (s, 1H Ar), 6.22 (broad s, 2H, NH₂), 6.34 (s, 1H Ar), 6.95-7.02 (t, 1H Ar), 7.20-7.24 (d, 1H Ar), 7.50-7.54 (d, 1H Ar), 7.62 (s, 1H Ar), 9.70 (s, 1H exchangeable, NH).

3-(7,8-dimethoxy-1-propyl-2-oxo-2,3-dihydro-1H-1,4-benzodiazepin-5-yl)benzamide, 5f

By replacing 3-(7,8-dimethoxy-1-methyl-2-oxo-2,3-dihydro-1H-1,4-benzodiazepin-5-yl)benzonitrile 4a in example 5a by 3-(7,8-dimethoxy-1-propyl-2-oxo-2,3-dihydro-1H-1,4-benzodiazepin-5-yl)benzonitrile 41 and proceeding in the same manner, the above product was obtained. Yield: 50%. ¹H NMR (DMSO-d₆, 200 MHz): d 0.61 (t, 3H, CH₃), 1.28-1.42 (m, 2H, CH₂CH3), 3.38-3.45 (m, 1H, NCH₂), 3.64 (s, 3H, OCH₃), 3.92 (s, 3H, OCH₃), 4.16 (AB system, ?d=0.79, J_{AB}=10 Hz, 2H, CH₂), 4.21-4.27 (m, 1H, NCH₂), 6.68 (s, 1H Ar), 7.19 (s, 1H Ar), 7.43-7.58 (m, 2H, 1H NH₂, 1H Ar), 7.69-7.74 (d, 1H Ar), 8.00-8.17 (m, 3H, 1H NH₂, 2H Ar).

3-(1-ethyl-7,8-dimethoxy-2-oxo-2,3-dihydro-1H-1,4-benzodiazepin-5-yl)benzamide, 5g

By replacing 3-(7,8-dimethoxy-1-methyl-2-oxo-2,3-dihydro-1H-1,4-benzodiazepin-5-yl)benzonitrile 4a in example 5a by 3-(7,8-dimethoxy-1-ethyl-2-oxo-2,3-dihydro-1H-1,4-benzodiazepin-5-yl)benzonitrile 4k and proceeding in the same manner, the above product was obtained. Yield: 53%. ¹H NMR (DMSO-d₆, 200 MHz): d 0.96 (t, 3H, CH₃), 3.33-3.42 (m, 1H, NCH₂), 3.64 (s, 3H, OCH₃), 3.92 (s, 3H, OCH₃), 4.14 (AB system, ?d=0.79, J_{AB}=10 Hz, 2H, CH₂), 4.21-4.28 (m, 1H, NCH₂), 6.67 (s, 1H Ar), 7.16 (s, 1H Ar), 7.43 (s, 1H NH₂ exchangeable), 7.50-7.58 (t, 1H Ar), 7.71-7.75 (d, 1H Ar), 8.00-8.14 (m, 2H, 1H NH₂, 1H Ar).

3-(1-benzyl-7,8-dimethoxy-2-oxo-2,3-dihydro-1H-1,4-benzodiazepin-5-yl)benzanide, 5h

By replacing 3-(7,8-dimethoxy-1-methyl-2-oxo-2,3-dihydro-1H-1,4-benzodiazepin-5-yl)benzonitrile 4a in example 5a by 3-(1-benzyl-7,8-dimethoxy-2-oxo-2,3-dihydro-1H-1,4-benzodiazepin-5-yl)benzonitrile 4 m and proceeding in the same manner, the above product was obtained. Yield: 38%.¹H NMR (CDCl₃, 300 MHz): d 3.68 (s, 3H, OCH₃), 3.87 (s, 3H, OCH₃), 4.42 (AB system, ?d=0.96, J_{AB}=10 Hz, 2H, CH₂), 5.16 (AB system, ?d=0.73 J_{AB}=15 Hz, 2H, NCH₂), 5.66 (s, 1H NH₂), 6.17 (s, 1H NH₂), 6.51 (s, 1H Ar), 7.09-7.19 (m, 6H Ar), 7.46-7.49 (d, 2H Ar), 7.89 (s, 1H Ar), 7.99-8.01 (d, 1H, 1H Ar).

Ethyl{5-[3-(aminocarbonyl)phenyl]-7,8-dimethoxy-2-oxo-2,3-dihydro-1H-1,4-benzodiazepin-1-yl}acetate, 5i

By replacing 3-(7,8-dimethoxy-1-methyl-2-oxo-2,3-dihydro-1H-1,4-benzodiazepin-5-yl)benzonitrile 4a in example 5a by ethyl[5-(3-cyanophenyl)-7,8-dimethoxy-2-oxo-2,3-dihydro-1H-1,4-benzodiazepin-1-yl]acetate 4n and proceeding in the same manner, the above product was obtained. Yield: 44%. ¹H NMR (CDCl₃, 300 MHz): d 1.22-1.27 (t, 3H CH₃), 3.75 (s, 3H, OCH₃), 3.96 (s, 3H, OCH₃), 4.15-4.24 (m, 2H CH₂—CH₃), 4.38 (AB system, ?d=0.90, J_{AB}=10 Hz, 2H, CH₂), 4.50-4.57 (m, 2H NCH₂), 5.70 (s, 1H NH₂), 6.65 (s, 1H NH₂), 6.85 (s, 1H Ar), 7.50-7.55 (t, 1H Ar), 7.80-7.82 (d, 1H Ar), 7.97-7.99 (d, 1H Ar), 8.17 (s, 1H Ar).

3-(7,8-dimethoxy-1,3-dimethyl-2-oxo-2,3-dihydro-1H-1,4-benzodiazepin-5-yl) benzamide, 5j

By replacing 3-(7,8-dimethoxy-1-methyl-2-oxo-2,3-dihydro-.1H-1,4-benzodiazepin-5-yl)benzonitrile 4a in example 5a by 3-(7,8-dimethoxy-1,3-dimethyl-2-oxo-2,3-dihydro-1H-1,4-benzodiazepin-5-yl)benzonitrile 7d and proceeding in the same manner, the above product was obtained. Yield: 44%. ¹H NMR (CDCl₃, 200 MHz): d 1.75-1.78 (d, 3H CH₃), 3.46 (s, 3H, NCH₃), 3.76-3.80 (m, 4H, CH and OCH₃), 4.01 (s, 3H OCH₃), 5.68 (s, 1H exchangeable NH), 7.18 (s, 1H exchangeable NH), 6.72 (s, 1H Ar), 6.82 (s, 1H Ar), 7.48-7.56 (t, 1H Ar), 7.79-7.81 (d, 1H Ar), 7.93-7.95 (d, 1H Ar), 8.15 (s, 1H Ar).

3-[3-(3,4-dichlorobenzyl)-7,8-dimethoxy-1-methyl-2-oxo-2,3-dihydro-1H-1,4-benzodiazepin-5-yl]benzaniide, 5k

By replacing 3-(7,8-dimethoxy-1-methyl-2-oxo-2,3-dihydro-1H-i ,4-benzodiazepin-5-yl)benzonitrile 4a in example Sa by 7,8-dimethoxy-1,3-dimethyl-5-(3-trifluoromethylphenyl)-1,3-dihydro-2H-1,4-benzodiazepin-2-one 7c and proceeding in the same manner, the above product was obtained. Yield: 58%. ¹H NMR (CDCl₃, 200 MHz): d 3.42 (s, 3H, NCH₃), 3.50-3.54 (m, 2H CH₂), 3.71 (s, 4H, CH and OCH₃), 3.97 (s, 3H OCH₃), 6.01 (s, 1H exchangeable NH), 6.15 (s, 1H exchangeable NH), 6.60 (s, 1H Ar), 6.77 (s, 1H Ar), 7.21-7.50 (m, 4H Ar), 7.76-7.80 (d, 1H Ar), 7.89-7.92 (d, 1H Ar), 8.03 (s, 1H Ar).

3-(8-methoxy-1-methyl-2-oxo-2,3-dihydro-1H-1,4-benzodiazepin-5-yl)benzamide, 51

By replacing 3-(7,8-dimethoxy-1-methyl-2-oxo-2,3-dihydro-1H-1,4-benzodiazepin-5-yl)benzonitrile 4a in example 5a by 3-(8-methoxy-1-methyl-2-oxo-2,3-dihydro-1H-1,4-benzodiazepin-5-yl)benzonitrile 29a and proceeding in the same manner, the above product was obtained. Yield: 60%. ¹H NMR (CDCl₃, 300 MHz): d 3.43 (s, 3H, NCH₃), 3.92 (s, 3H, OCH₃), 4.32 (AB system, ?d=0.99 J_{AB}=10 Hz, 2H, CH₂), 5.70 (s, 1H exchangeable NH), 6.28 (s, 1H exchangeable NH), 6.75-6.78 (d, 1H Ar), 6.84 (s, 1H Ar), 7.19-7.22 (d, 1H Ar), 7.47-7.52 (t, 1H Ar), 7.76-7.78 (d, 1H Ar), 7.94-7.96 (d, 1H Ar), 8.09 (s, 1H Ar).

3-(7,8-dimethoxy-1-methyl-2-oxo-9-phenyl-2,3-dihydro-1H-1,4-benzodiazepin-5-yl)benzamide, 5m

By replacing 3-(7,8-dimethoxy-1-methyl-2-oxo-2,3-dihydro-1H-1,4-benzodiazepin-5-yl)benzonitrile 4a in example 5a by 3-(7,8-dimethoxy-1-methyl-2-oxo-9-phenyl-2,3-dihydro-1H-1,4-benzodiazepin-5-yl)benzonitrile 25a and proceeding in the same manner, the above product was obtained. Yield: 75%. 1H NMR (CDCl₃, 200 MHz): d 2.44 (s, 3H, NCH₃), 3.62 (s, 3H, OCH₃), 3.77 (s, 3H, OCH₃),4.41 (AB system, ?d=0.5, J_{AB}=10 Hz, 2H, NCH₂), 6.74 (s, 1H Ar), 7.44-7.57 (m, 6H Ar), 7.88-7.98 (m, 2H Ar), 8.25 (s, 1H Ar), 10.19 (s, 2H, NH₂).

3-(6,8-dimethoxy-2-oxo-2,3-dihydro-1H-1,4-benzodiazepin-5-yl)benzamide, 5n

By replacing 3-(7,8-dimethoxy-1-methyl-2-oxo-2,3-dihydro-1H-1,4-benzodiazepin-5-yl)benzonitrile 4a in example 5a by 3-(6,8-dimethoxy-2-oxo-2,3-dihydro-1H-1,4-benzodiazepin-5-yl)benzonitrile 28g and proceeding in the same manner, the above product was obtained. Yield: 80%. ¹H NMR (DMSO-d₆, 300 MHz): d 3.42 (s, 3H, OCH₃), 3.85 (s, 3H, OCH₃),4.17 (AB system, ?d=0.7, J_{AB}=11 Hz, 2H, NCH₂), 6.44-6.45 (m, 2H Ar), 7.35-7.88 (m, 4H Ar), 8.03 (s, 2H, NH₂), 10.39 (s, 1H, NH).

3-(6,8-dimethoxy-1-methyl-2-oxo-2,3-dihydro-1H-1,4-benzodiazepin-5-yl)benzamide, 5o

By replacing 3-(7,8-dimethoxy-1-methyl-2-oxo-2,3-dihydro-1H-1,4-benzodiazepin-5-yl)benzonitrile 4a in example 5a by 3-(6,8-dimethoxy-1-methyl-2-oxo-2,3-dihydro-1H-1,4-benzodiazepin-5-yl)benzonitrile 29b and proceeding in the same manner, the above product was obtained. Yield: 73%. ¹H NMR (CDCl₃, 300 MHz): d 3.37 (s, 3H, NCH₃), 3.48 (s, 3H, OCH₃), 3.90 (s, 3H, OCH₃),4.28 (AB system, ?d=0.8, J_{AB}=9 Hz, 2H, NCH₂), 6.33 (s, 2H Ar), 6.44 (s, 2H Ar), 7.39-8.01 (m, 4H Ar).

Palladium couplings of type 6.

Tert-butyl-3-[3-(7,8-dimethoxy-1-methyl-2-oxo-2,3-dihydro-1H-1,4-benzodiazepin-5-yl)phenyl]propynylcarbamate, 6a

A mixture of 100 mg (0.257 mmole) of 5-(3-bromophenyl)-7,8-dimethoxy-1-methyl-1,3-dihydro-2H-1,4-benzodiazepin-2-one 4b, 200 mg (1.3 mmoles) of tert-butyl prop-2-ynylcarbamate, 9.0 mg of CuI, 5.0 mg of PdCl₂ 18.0 mg of PPh₃, 0.5 ml of TEA, 2 ml of CH₃CN was stirred for 20 hours in an inert atmosphere at 50° C. The mixture was evaporated to dryness and purified by chromatography on silica gel (Et2O/CH₂Cl₂, 1:1). Recrystallization was in Et₂O/pentane. Yield: 50%. ¹H NMR (CDCl₃, 200 MHz): d 1.47 (s, 9H, C(CH₃)₃), 3.41 (s, 3H, NCH₃), 3.76 (s, 3H, OCH₃), 3.99 (s, 3H, OCH₃), 4.20 (AB system, ?d=1.0, J_{AB}=10 Hz, 2H, NCH₂), 4.12-4.15 (d, 2H, CH₂), 6.65 (s, 1H Ar), 6.79 (s, 1H Ar), 7.47-7.73 (m, 4H Ar).

7,8-dimethoxy-5-(3 ′-hex-1-ynylphenyl)-1-N-methyl-1,3-dihydro-2H-1,4-benzodiazepin-2-one, 6b

By replacing tert-butyl prop-2-ynylcarbamate in example 6a by 1-hexyne and proceeding in the same manner, the above product was obtained. Yield: 89%. ¹H NMR (CDCl₃, 200 MHz): d 0.91-0.98 (t, 3H, CH₃), 1.40-1.60 (m, 4H, 2 CH₂), 2.36-2.43 (t, 2H, CH₂), 3.40 (s, 3H, NCH₃), 3.76 (s, 3H, OCH₃), 3.99 (s, 3H, OCH₃), 4.20 (AB system, ?d=1.0, J_{AB}=11 Hz, 2H, NCH₂), 6.67 (s, 1H Ar), 6.78 (s, 1H Ar), 7.29-7.36 (t, 1H Ar), 7.46-7.50 (d, 1H Ar), 7.54-7.58 (d, 1H Ar), 7.65 (d, 1H Ar).

7,8-dimethoxy-l-methyl-5-[3-(3-piperidin-1-ylprop-1-ynyl)phenyl]-1,3-dihydro-2H-1,4-benzodiazepin-2-one, 6c

By replacing tert-butyl prop-2-ynylcarbamate in example 6a by propargyl bromide, PdCl₂ by Pd(OAc)₂, TEA by piperidine and CH₃CN by THF, and proceeding in the same manner, the above product was obtained. Yield: 20%. ¹H NMR (CDCl₃, 200 MHz): d 1.48 (m, 2H, CH₂), 1.62-1.65 (m, 4H, 2 CH₂), 2.56 (m, 4H, 2 CH₂), 3.41 (s, 3H, NCH₃), 3.46 (s, 2H, CH₂), 3.76 (s, 3H, OCH₃), 3.99 (s, 3H, OCH₃), 4.20 (AB system, ?d=1.0, J_{AB}=11 Hz, 2H, NCH₂), 6.67 (s, 1H Ar), 6.78 (s, 1H Ar), 7.34-7.71 (m, 4H Ar).

6-[3-(7,8-dimethoxy-1-methyl-2-oxo-2,3-dihydro-1H-1,4-benzodiazepin-5-yl)phenyl]hex-5-ynenitrile, 6d

By replacing tert-butyl prop-2-ynylcarbamate in example 6a by 5-cyano-1-pentyne and proceeding in the same manner, the above product was obtained. Yield: 81%. ¹H NMR (CDCl₃, 300 MHz): d 1.94-2.01 (m, 2H, CH₂), 2.54-2.63 (m, 4H, 2 CH₂), 3.41 (s, 3H, NCH₃), 3.76 (s, 3H, OCH₃), 3.99 (s, 3H, OCH₃), 4.20 (AB system, ?d=1.0, J_{AB}=11 Hz, 2H, NCH₂), 6.66 (s, 1H Ar), 6.79 (s, 1H Ar), 7.32-7.37 (t, 1H Ar), 7.47-7.50 (d, 1H Ar), 7.56-7.59 (d, 1H Ar), 7.69 (d, 1H Ar).

7,8-dimethoxy-5-(3 ′-hexylphenyl)-1-N-methyl-1,3-dihydro-2H-1,4-benzodiazepin-2-one, 6e

A mixture of 68 mg (0.172 mmole) of 7,8-dimethoxy-5-(3′-hex-1-ynylphenyl)-1-N-methyl-1,3-dihydro-2H-1,4-benzodiazepin-2-one 6b, 20 mg of Pd/C 10% by weight in 10 ml of MeOH and 2 ml of CH₂Cl₂ was stirred under H₂ at atmospheric pressure for 3 hours. The suspension was filtered on celite, washed with 3×10 ml of CH₂CL₂/MeOH 8:2, evaporated to dryness and purified by silica gel chromatography (CH₂Cl₂/Et₂O, 1:1). Yield: 65%. ¹H NMR (CDCl₃, 300 MHz): d 0.88 (m, 3H, CH₃), 1.31 (m, 8H, 4 CH₂), 2.60-2.65 (t, 2H, CH₂), 3.40 (s, 3H, NCH₃), 3.74 (s, 3H, OCH₃), 3.98 (s, 3H, OCH₃), 4.20 (AB system, ?d=1.0, J_{AB}=11 Hz, 2H, NCH₂), 6.71 (s, 1H Ar), 6.78 (s, 1H Ar), 7.28-7.49 (m, 4H Ar).

Tert-butyl-3-[3-(7,8-dimethoxy-1-methyl-2-oxo-2,3-dihydro-1H-1,4-benzodiazepin-5-yl)phenyl]propylcarbamate, 6f

By replacing 7,8-dimethoxy-5-(3′-hex-1-ynylphenyl)-1-N-methyl-1,3-dihydro-2H-1,4-benzodiazepin-2-one 6b in example 6e by tert-butyl-3-[3-(7,8-dimethoxy-1-methyl-2-oxo-2,3-dihydro-1H-1,4-benzodiazepin-5-yl)phenyl]propynylcarbamate 6a and proceeding in the same manner, the above product was obtained. Yield: 58%. ¹H NMR (CDCl₃, 200 MHz): d 1.44 (s, 9H, C(CH₃)₃), 1.81 (m, 2H, CH₂), 2.63-2.70 (m, 2H, CH₂), 3.14-3.22 (m, 2H, CH₂), 3.41 (s, 3H, NCH₃), 3.75 (s, 3H, OCH₃), 3.99 (s, 3H, OCH₃), 4.30 (AB system, ?d=1.0, J_{AB}=10 Hz, 2H, NCH₂), 4.56 (broad s, 1H, NH), 6.70 (s, 1H Ar), 6.78 (s, 1H Ar), 7.26-7.69 (m, 4H Ar).

6-[3-(7,8-dimethoxy-1-methyl-2-oxo-2,3-dihydro-1H-1,4-benzodiazepin-5-yl)phenyl]hexanenitrile, 6g

By replacing 7,8-dimethoxy-5-(3′-hex-1-ynylphenyl)-1-N-methyl-1,3-dihydro-2H-1,4-benzodiazepin-2-one 6b in example 6e by 6-[3-(7,8-dimethoxy-1-methyl-2-oxo-2,3-dihydro-1H-1,4-benzodiazepin-5-yl)phenyl]hex-5-ynenitrile 6d and proceeding in the same manner, the above product was obtained. Yield: 60%. ¹H NMR (CDCl₃, 200 MHz): d 1.44-1.70 (m, 6H, 3 CH₂), 2.34 (m, 2H, CH₂), 2.67 (m, 2H, CH₂), 3.41 (s, 3H, NCH₃), 3.75 (s, 3H, OCH₃), 3.99 (s, 3H, OCH₃), 4.30 (AB system, ?d=1.0, J_{AB}=10 Hz, 2H, NCH₂), 6.71 (s, 1H Ar), 6.79 (s, 1H Ar), 7.28-7.53 (m, 4H Ar).

5-[3-(3-aniinopropyl)phenyl-7,8-dimethoxy-1-methyl-1,3-dihydro-2H-1,4-benzodiazepin-2-one trifluoroacetate, 6h

A mixture of 25 mg (0.05 mmole) of tert-butyl-3-[3-(7,8-dimethoxy-1-methyl-2-oxo-2,3-dihydro-1H-1,4-benzodiazepin-5-yl)phenyl]propylcarbamate 6f, trifluoroacetic acid (40 □1, 0.52 mmole) and CH₂Cl₂ was stirred for 3 hours under an inert atmosphere at room temperature, then evaporated to dryness. The product was crystallized in ether (13 mg). Yield: 50%. ¹H NMR (DMSO-d₆, 300 MHz): d 1.84 (m, 2H, CH₂), 2.67-2.69 (m, 2H, CH₂), 2.72-2.81 (m, 2H, CH₂), 3.34 (s, 3H, NCH₃), 3.68 (s, 3H, OCH₃), 3.93 (s, 3H, OCH₃), 4.15 (AB system, ?d=0.7, J_{AB}=11 Hz, 2H, NCH₂), 6.69 (s, 1H Ar), 7.13 (s, 1H Ar), 7.40-7.60 (m, 4H Ar), 7.83 (broad s, 3H, NH₃⁺).

6-[3-(7,8-dimethoxy-1-methyl-2-oxo-2,3-dihydro-1H-1,4-benzodiazepin-5-yl)phenyl]hexanamide, 6i

By replacing 3-(7,8-dimethoxy-1-methyl-2-oxo-2,3-dihydro-1H-1,4-benzodiazepin-5-yl)benzonitrile 4a in example 5a by 6-[3-(7,8-dimethoxy-1-methyl-2-oxo-2,3-dihydro-1H-1,4-benzodiazepin-5-yl)phenyl]hexanenitrile 6g and proceeding in the same manner, the above product was obtained. Yield: 50%. ¹H NMR (CDCl₃, 200 MHz): d 1.37 (m, 2H, CH₂), 1.62 (m, 4H, 2 CH₂), 2.17-2.21 (m, 2H, CH₂), 2.60-2.64 (m, 2H, CH₂), 3.40 (s, 3H, NCH₃), 3.74 (s, 3H, OCH₃), 3.98 (s, 3H, OCH₃), 4.30 (AB system, ?d=1.0, J_{AB}=11 Hz, 2H, NCH₂), 5.46 (broad s, 2H, NH₂), 6.70 (s, 1H Ar), 6.78 (s, 1H Ar), 7.28-7.50 (m, 4H Ar).

5-(4′-chloro-1,1 ′-biphenyl-3-yl)-7,8-dimethoxy-1-methyl-1,3-dihydro-2H-1,4-benzodiazepin-2-one, 6j

By replacing 5-chloro-7,8-dimethoxy-1-methyl-1,3-dihydro-1,4-benzodiazepin-2-one 16a in example 17a by 5-(3-bromophenyl)-7,8-dimethoxy-1-methyl-1,3-dihydro-2H-1,4-benzodiazepin-2-one 4b and 3-chlorobenzene boronic acid by 4-chlorobenzene boronic acid, and proceeding in the same manner, the above product was obtained. Yield: 27%. MP: 131° C. ¹H NMR (DMSO, 300 MHz): d 3.32 (s, 3H, NCH₃), 3.63 (s, 3H, OCH₃), 3.91 (s, 3H, OCH₃), 4.16 (AB system, ?d=1.0, J_{AB}=10, 2H, CH₂), 6.74 (s, 1H Ar), 7.10 (s, 1H Ar), 7.51-7.80 (m, 8H Ar).

5-{3-[3-(benzyloxy)prop-1-ynyl]phenyl}-1-ethyl-7,8-dimethoxy-1,3-dihydro-2H-1,4-benzodiazepin-2-one, 6k

By replacing tert-butyl prop-2-ynylcarbamate in example 6a by [(prop-2-ynyloxy)methyl]benzene and proceeding in the same manner, the above product was obtained. Yield: 24%. MP: ° C. ¹H NMR (CDCl₃, 200MHz): d 1.12 (s, 3H, CH₃), 3.50-3.72 (m, 7H, 1H NCH₂+2H OCH₂Ph+1H CH₂ +OCH₃), 3.97 (s, 3H, OCH₃), 4.24-4.38 (m, 2H, 1H =C—CH₂+1H NCH₂), 4.66 (s, 1H, =C—CH₂), 4.73-4.78 (m, 1H CH₂), 6.63 (s, 11H Ar), 6.84 (s. 1H Ar), 7.26-7.83 (m, 9H Ar).

3′-(7,8-dimethoxy-1-methyl-2-oxo-2,3-dihydro-1H-1,4-benzodiazepin-5-yl)-1,1′-biphenyl-3-carbonitrile, 6l

By replacing 5-chloro-7,8-dimethoxy-1-methyl-1,3-dihydro-1,4-benzodiazepin-2-one 16a in example 17a by 5-(3-bromophenyl)-7,8-dimethoxy-1-methyl-1,3-dihydro-2H-1,4-benzodiazepin-2-one 4b and 3-chlorobenzene boronic acid by 3-cyanobenzene boronic acid, and proceeding in the same manner, the above product was obtained. Yield: 54%. ¹H NMR (CDCl3, 200 MHz): d 3.43 (s, 3H, NCH₃), 3.76 (s, 3H, OCH₃), 4.00 (s, 3H, OCH₃), 4.33 (AB system, ?d=1.00, J_{AB}=10, 2H, CH₂), 6.72 (s, 1H Ar), 6.81 (s, 1H Ar), 7.47-8.00 (m, 8H Ar).

3′-(7,8-dimethoxy-1-methyl-2-oxo-2,3-dihydro-1H-1,4-benzodiazepin-5-yl)-1,1′-biphenyl-4-carbonitrile, 6m

By replacing 5-chloro-7,8-dimethoxy-1-methyl-1,3-dihydro-1,4-benzodiazepin-2-one 16a in example 17a by 5-(3-bromophenyl)-7,8-dimethoxy-1-methyl-1,3-dihydro-2H-1,4-benzodiazepin-2-one 4b and 3-chlorobenzene boronic acid by 4-cyanobenzene boronic acid, and proceeding in the same manner, the above product was obtained. Yield: 42%. ¹H NMR (CDCl3, 200 MHz): d 3.42 (s, 3H, NCH₃), 3.75 (s, 3H, OCH₃), 4.00 (s, 3H, OCH₃), 4.33 (AB system, ?d=1.00, J_{AB}=10.29, 2H, CH₂), 6.73 (s, 1H Ar), 6.80 (s, 1H Ar), 7.40-7.70 (m, 7H Ar), 7.97 (s, 1H Ar).

3′-(7,8-dimethoxy-1-methyl-2-oxo-2,3-dihydro-1H-1,4-benzodiazepin-5-yl)-1,1′-biphenyl4-carboxamide, 6n

By replacing 3-(7,8-dimethoxy-1-methyl-2-oxo-2,3-dihydro-1H-1,4-benzodiazepin-5-yl)benzonitrile 4a in example 5a by 3′-(7,8-dimethoxy-1-methyl-2-oxo-2,3-dihydro-1H-1,4-benzodiazepin-5-yl)-1,1′-biphenyl-3-carbonitrile 61 and proceeding in the same manner, the above product was obtained. Yield: 44%. ¹H NMR (CDCl₃, 300 MHz): d 3.44 (s, 3H, NCH₃), 3.77 (s, 3H, OCH₃), 4.01 (s, 3H, OCH₃), 4.35 (AB system, ?d =1.00, J_{AB}=10, 2H, CH₂), 5.66 (s, 1H exchangeable NH₂), 6.08 (s, 1H exchangeable NH₂), 6.76 (s, 1H Ar), 6.82 (s, 1H Ar), 7.48-7.74 (m, 5H Ar), 7.89-7.92 (d, 2H Ar), 7.99 (s, 1H Ar).

3′-(7,8-dimethoxy-1-methyl-2-oxo-2,3-dihydro-1H-1,4-benzodiazepin-5-yl)-1,1′-biphenyl-3-carboxamide, 6o

By replacing 3-(7,8-dimethoxy-1-methyl-2-oxo-2,3-dihydro-1H-1,4-benzodiazepin-5-yl)benzonitrile 4a in example 5a by 3′-(7,8-dimethoxy-1-methyl-2-oxo-2,3-dihydro-1H-1,4-benzodiazepin-5-yl)-1,1 ′-biphenyl-4-carbonitrile 6m and proceeding in the same manner, the above product was obtained. Yield: 44%. ¹H NMR (CDCl₃, 300 MHz): d 3.44 (s, 3H, NCH₃), 3.77 (s, 3H, OCH₃), 4.01 (s, 3H, OCH₃), 4.34 (AB system, ?d=1.00, J_{AB}=10, 2H, CH₂), 5.65 (s, 1H exchangeable NH₂), 6.19 (s, 1H exchangeable NH₂), 6.76 (s, 1H Ar), 6.82 (s, 1H Ar), 7.47-7.62 (m, 4H Ar), 7.71-7.80 (m, 3H Ar), 7.99 (s, 1H Ar).

Alkylation of carbon 3 of type 7.

3-(3,4-dichlorobenzyl)-l-ethyl-7,8-dimethoxy-5-phenyl-1,3-dihydro-2H-1,4-benzodiazepin-2-one, 7a

LDA (2 M, 0.31 ml, 0.62 mmole) was placed at −78° C. under argon and stirred, then 7,8-dimethoxy-1-ethyl-5-phenyl-1,3-dihydro-2H-1,4-benzodiazepin-2-one 4o (100 mg, 0.31 mmole) dissolved in THF (4 ml) was added dropwise. The reaction was returned to 0° C. for 30 minutes, then put back at −78° C. for addition of 3,4-dichlorobenzyl (81.5 mg, 0.35 mmole). The reaction was returned to room temperature and stirred overnight. It was quenched with saturated NaCl (15 ml) and extracted with 3×10 ml of dichloromethane. The organic phases were pooled and washed with 30 ml of water, dried on Na₂SO₄, filtered, evaporated to dryness and purified by silica gel chromatography (EtOAc/Hex, 1:1) to give the above product. Yield: 53%. ¹H NMR (CDCl₃, 200 MHz): d 1.03-1.11 (t, 3H, CH₃), 3.50-3.68 (m, 4H, CH, CH₂ and 1H NCH₂), 3.73 (s, 3H, OCH₃), 3.96 (s, 3H, OCH₃), 6.63 (s, 1H Ar), 6.82 (s, 1H Ar), 7.19-7.55 (m, 8H Ar).

3-[3-(3,4-dichlorobenzyl)-7,8-dimethoxy-1-methyl-2-oxo-2,3-dihydro-1H-1,4-benzodiazepin-5-yl]benzonitrile, 7b

By replacing 7,8-dimethoxy-1-ethyl-5-phenyl-1,3-dihydro-2H-1,4-benzodiazepin-2-one 4o in example 7a by 3-(7,8-dimethoxy-2-oxo-2,3-dihydro-1H-1,4-benzodiazepin-5-yl)-benzonitrile 3a and proceeding in the same manner, the above product was obtained. Yield: 53%. ¹H NMR (CDCl₃, 200 MHz): d 3.46 (s, 3H, NCH₃), 3.50-3.57 (m, 2H, CH and 1H CH₂), 3.72-3.78 (m, 4H, 1H CH₂ and OCH₃), 4.01 (s, 3H OCH₃), 6.60 (s, 1H Ar), 6.82 (s, 1H Ar), 7.21-7.26 (m, 1H Ar), 7.38-7.42 (m, 1H Ar), 7.50-7.60 (m, 2H Ar), 7.75-7.80 (d, 1H Ar), 7.88-7.91 (m, 2H Ar).

7,8-dimethoxy-1,3-dimethyl-5-(3-trifluoromethylphenyl)-1,3-dihydro-2H-1,4-benzodiazepin-2-one, 7c

By replacing 3,4-dichlorobenzyl in example 7a by iodomethane and proceeding in the same manner, the above product was obtained. Yield: 45%. ¹H NMR (CDCl₃, 200 MHz): d 1.62-1.79 (d, 3H CH₃), 3.40 (s, 3H, NCH₃), 3.78-3.80 (m, 4H, CH and OCH₃), 4.03 (s, 3H OCH₃), 6.68 (s, 1H Ar), 6.84 (s, 1H Ar), 7.52-7.60 (t, 1H Ar), 7.72-7.77 (d, 1H Ar), 7.90-7.95 (m, 2H Ar).

3-(7,8-dimethoxy-1,3-dimethyl-2-oxo-2,3-dihydro-1H-1,4-benzodiazepin-5-yl)benzonitrile, 7d

By replacing 7,8-dimethoxy-1-ethyl-5-phenyl-1,3-dihydro-2H-1,4-benzodiazepin-2-one 4o in example 7a by 3-(7,8-dimethoxy-2-oxo-2,3-dihydro-1H-1,4-benzodiazepin-5-yl)-benzonitrile 3a, and 3,4-dichlorobenzyl by iodomethane, and proceeding in the same manner, the above product was obtained. Yield: 53%. ¹H NMR (CDCl₃, 200 MHz): d 1.72-1.75 (d, 3H CH₃), 3.42 (s, 3H, NCH₃), 3.75-3.77 (m, 4H, CH and OCH₃), 3.99 (s, 3H OCH₃), 6.61 (s, 1H Ar), 6.80 (s, 1H Ar), 7.48-7.56 (t, 1H Ar), 7.71-7.75 (d, 1H Ar), 7.90-7.97 (m, 2H Ar).

Reduction of the nitrile of type 8.

5-[3-(aminomethyl)phenyl]-7,8-dimethoxy-1-methyl-1,3-dihydro-2H-1,4-benzodiazepin-2-one, 8a

3-(7,8-dimethoxy-1-methyl-2-oxo-2,3-dihydro-1H-1,4-benzodiazepin-5-yl)-benzamide 5a (100 mg, 0.30 mmole), Raney nickel (1 spatula tip), ammonia (30%, 1 ml) and methanol (10 ml) were placed overnight under hydrogen pressure, then filtered on celite and washed with 3×25 ml of methanol and evaporated. The residue was taken up in 20 ml of CH₂Cl₂. The organic phase was washed with 3×20 ml of 30% ammonia and 1×20 ml of water. The organic phase was dried on Na₂SO₄, filtered and evaporated to dryness to give the above product. Yield: 77%. ¹H NMR (CDCl₃, 200 MHz): d 1.28 (s, 2H exchangeable NH₂), 1.79 (s, 2H CH₂), 3.42 (s, 3H, NCH₃), 3.76 (s, 3H, OCH₃), 3.94 (s, 2H CH₂), 4.00 (s, 3H OCH₃), 4.31 (AB system, ?d=0.98, J_{AB}=10, 2H, CH₂), 6.72 (s, 1H Ar), 6.81 (s, 1H Ar), 7.36-7.56 (m, 3H Ar), 7.71 (s, 1H Ar).

N-[3-(7,8-dimethoxy-1-methyl-2-oxo-2,3-dihydro-1H-1,4-benzodiazepin-5-yl)benzyl]acetamide, 8b 5-[3-(aminomethyl)phenyl]-7,8-dimethoxy-1-methyl-1,3-dihydro-2H-1,4-benzodiazepin-2-one 8a (50 mg, 0.15 mmole), acetic anhydride (16 μl, 0.18 mmole), pyridine (29.6 ptl, 0.37 mmole), and 3 ml of CH₂Cl₂ were stirred overnight, evaporated to dryness and purified by chromatography on silica gel (CH₂Cl₂/MeOH, 9:1) to give the above product. Yield: 77%. ¹H NMR (CDCl₃, 200 MHz): d 2.00 (s, 3H CH₃), 3.38 (s, 3H, NCH₃), 3.76 (s, 3H, OCH₃), 3.94 (s, 3H OCH₃), 4.25 (AB system, ?d=0.98, J_{AB}=10, 2H, CH₂), 4.43 (s, 2H CH₂), 5.99 (s, 1H exchangeable NH), 6.65 (s, 1H Ar), 6.77 (s, 1H Ar), 7.26-7.40 (m, 3H Ar), 7.63 (s, 1H Ar).

Synthesis of thiobenzamides of type 9

3-(7,8-dimethoxy-1-methyl-2-oxo-2,3-dihydro-1H-1,4-benzodiazepin-5-yl)thiobenzamide, 9a

A mixture of 500 mg (1.41 mmoles) of 3-(7,8-dimethoxy-1-methyl-2,3-dihydro-1H-1,4-benzodiazepin-5-yl)benzamide 5a, 285 mg (0.71 mmole) of Lawesson reagent in 30 ml of toluene was heated overnight at 90° C. 150 ml of H₂O were added and the mixture was extracted with 4×100 ml of EtOAc, dried on MgSO₄, the EtOAc was evaporated and the product was purified by silica gel chromatography (EtOAc/CH₂Cl₂/EtOH, 5:4:1). Yield: 70%. ¹H NMR (CDCl₃, 300 MHz): d 3.41 (s, 3H, NCH₃), 3.77-3.84 (m, 4H, 1H CH₂+OCH₃), 3.99 (s, 3H, OCH₃), 4.81 (m, 1H, CH₂), 6.68 (s, 1H Ar), 6.79 (s, 1H Ar), 7.40-8.20 (m, 6H, NH₂+4H Ar). Mass: (M+H)⁺=370.09.

7,8-dimethoxy-1-methyl-5-[3-(4-phenyl-1,3-thiazol-2-yl)phenyl]-1,3-dihydro-2H-1,4-benzodiazepin-2-one, 9b

A mixture of 20 mg (0.05 mmole) of 3-(7,8-dimethoxy-1,3-dihydro-2H-1,4-benzodiazepin-5-yl)thiobenzamide 9a, 12 mg (0.06 mmole) of bromoacetophenone in 3 ml of EtOH was heated overnight at 100° C. 50 ml of H₂O were added and the mixture was extracted with 4×50 ml of EtOAc, dried on MgSO₄, the EtOAc was evaporated and the product was purified by silica gel chromatography (EtOAc/Hex, 1:1). Yield: 90%. ¹H NMR (CDCl₃, 300 MHz): d 3.45 (s, 3H, NCH₃), 3.77 (s, 3H, OCH₃), 4.01 (s, 3H, OCH₃), 4.36 (AB system, ?d=1,02, J_{AB}=10.5, 2H, CH₂), 6.76 (s, 1H Ar), 6.82 (s, 1H Ar), 7.44-8.00 (m, 9H Ar), 8.34 (s, 1H thiazol). Mass: (M+H)⁺=470.14.

Synthesis of benzodiazepine-thiones of type 10

7,8-dimethoxy-5-phenyl-1,3-dihydro-2H-1,4-benzodiazepine-2-thione, 10a

7,8-dimethoxy-5-phenyl-1,3-dihydro-2H-1,4-benzodiazepin-2-one 3c (400 mg, 1.35 mmoles), 600 mg of Lawesson reagent and 70 ml of anhydrous toluene were refluxed overnight, then evaporated to dryness and purified by chromatography on silica gel (EtOAc) to give the above product. Yield: 85%. ¹H NMR (CDCl₃, 200 MHz): d 3.68 (s, 3H, OCH₃), 3.98 (s, 3H OCH₃), 4.77 (s, 2H, CH₂), 6.46 (s, 1H Ar), 7.20 (s, 1H Ar), 7.36-7.40 (m, 2H Ar), 7.54-7.60 (m, 3H Ar), 13.02 (s, 1H exchangeable NH).

7,8-diethoxy-5-phenyl-1,3-dihydro-2H-1,4-benzodiazepine-2-thione, 10b

By replacing 7,8-dimethoxy-5-phenyl-1,3-dihydro-2H-1,4-benzodiazepin-2-one 3c in example 11a by 7,8-diethoxy-5-phenyl-1,3-dihydro-2H-1,4-benzodiazepin-2-one 3e and proceeding in the same manner, the above product was obtained. Yield: 53%. ¹H NMR (CDCl₃, 300 MHz): d 1.33-1.38 (t, 3H, CH₃), 1.50-1.54 (t, 3H, CH₃), 3.83-3.90 (q, 2H, CH₂), 4.15-4.22 (q, 2H, CH₂), 4.75 (s, 1H exchangeable NH), 6.47 (s, 1H Ar), 7.12 (s, 1H Ar), 7.36-7.39 (m, 2H Ar), 7.48-7.62 (m, 3H Ar).

1-ethyl-7,8-dimethoxy-5-phenyl-1,3-dihydro-2H-1,4-benzodiazepine-2-thione, 10c

By replacing 3-(7,8-dimethoxy-2-oxo-2,3-dihydro-1H-1,4-benzodiazepin-5-yl)-benzonitrile 3a in example 4a by 7,8-diethoxy-5-phenyl-1,3-dihydro-2H-1,4-benzodiazepine-2-thione 11b and iodomethane by iodoethane, and proceeding in the same manner, the above product was obtained. Yield: 75%. ¹H NMR (CDCl₃, 300 MHz): d 1.16-1.21 (t, 3H CH₃), 3.76 (s, 3H, OCH₃), 3.95-4.02 (m, 4H, OCH₃ and 1H NCH₂), 4.76 (AB system, ?d=1.22, J_{AB}=10 Hz, 2H, CH₂), 5.05-5.12 (m, 1H NCH₂), 6.68 (s, 1H Ar), 6.91 (s, 1H Ar), 7.42-7.48 (m, 3H Ar), 7.65-7.68 (m, 2H Ar).

5-(3-cyanophenyl)-7,8-dimethoxy-1,3-dihydro-2H-1,4-benzodiazepin-2-thione, 10d

A mixture of 200 mg (0.62 mmole) of 5-(3-cyanophenyl)-7,8-dimethoxy-1,3-dihydro-2H-1,4-benzodiazepin-2-one 3a, 150 mg (0.34 mmole) of P₂S₅ in 4 ml of pyridine was heated under reflux for 45 minutes, then cooled to 0° C. 100 ml of saturated NaCl was then added to the suspension, which was filtered, rinsed with cold water, vacuum dried, triturated in Et₂O, filtered and dried. Yield: 76%. ¹H NMR (CDCl₃, 300 MHz): d 3.76 (s, 3H, OCH₃), 3.99 (s, 3H, OCH₃), 4.78 (m, 2H, CH₂), 6.63 (s, 1H.Ar), 6.68 (s, 1H Ar), 7.50-7.92 (m, 3H, 4H Ar), 9.84 (s, 1H exchangeable, NH ). Mass: (M+H)⁺=338.10.

Nucleophilic substitution reaction (type 11 and 12) of amines on a thione.

3-(8,9-dimethoxy-4H-imidazo[1,2-a][1,4] benzodiazepin-6-yl)benzonitrile, 1 a

A mixture of 100 mg (0.30 mmole) of 5-(3-cyanophenyl)-7,8-dimethoxy-1,3-dihydro-2H-1,4-benzodiazepin-2-thione 10d, 40 mg (0.30 mmole) of aminoacetaldiethylacetal and 3 mg (0.015 mmole) of paratoluenesulfonic acid monohydrate in 5 ml of butanol was heated overnight under reflux. 55 mg (0.29 mmole) of paratoluenesulfonic acid monohydrate were added and the mixture was heated under reflux for 6 h. The butanol was evaporated to ⅔, 100 ml of ice H₂O was added and the solution was basified to pH 8.9 with 1N NaOH, then extracted with 3×100 ml of EtOAc and dried on MgSO₄. The EtOAc was evaporated and the product was purified by silica chromatography (EtOAc). Yield: 55%. ¹H NMR (CDCl₃, 200 MHz): d 3.77 (s, 3H, OCH₃), 4.02 (m, 4H, 1HCH₂+OCH₃), 5.33 (m, 1HCH₂), 6.68 (s, 1H Ar), 6.99 (s, 1H Ar), 7.13 (d, J₁₂=1.22, 1H-imidazol), 7.36 (d, J₂₁=1.22, 1H-imidazol), 7.48-7.84 (m, 3H, 4H Ar).

3-(8,9-dimethoxy-4H-imidazo[1,2-a] [1,4]benzodiazepin-6-yl)benzamide, 11b

By replacing 3-(7,8-dimethoxy-1-methyl-2-oxo-2,3-dihydro-1H-1,4-benzodiazepin-5-yl) benzonitrile 4a in example 5a by 3-(8,9-dimethoxy-4H-imidazo[1,2-a][1,4]benzodiazepin-6-yl)benzonitrile 11a and proceeding in the same manner, the above product was obtained. Yield: 70%. ¹H NMR (CDCl₃, 300 MHz): d 3.76 (s, 3H, OCH₃), 4.03 (m, 4H, 1HCH₂+OCH₃), 5.33 (m, ¹HCH₂), 6.53 (s, 1H Ar), 6.75 (s, 1H Ar), 6.99 (s, 1H-imidazol), 7.27 (s, 1H-imidazol), 7.42-8.09 (m, 3H, 4H Ar).

3-(7,8-dimethoxy-2-methylamino-1,3-dihydro-3H-1,4-benzodiazepin-5-yl)benzonitrile, 12a

1.5 ml of 2N methylamine in THF were added to a solution of 100 mg (0.30 mmole) of 5-(3-cyanophenyl)-7,8-dimethoxy-1,3-dihydro-2H-1,4-benzodiazepin-2-thione 10d in 3 ml of EtOH and 0.5 ml of DMSO, then heated overnight at 110° C. 100 ml of water H₂O were added and the mixture was extracted with 3×100 ml of CH₂Cl₂, dried on MgSO₄. The CH₂Cl₂ was evaporated and the product was purified by chromatography on silica gel (EtOAc; EtOAc/CH₂Cl₂/EtOH, 5:4:1). Yield: 30%. 1H NMR (DMSO, 300 MHz): d 2.96 (s, 3H, NCH₃), 3.59-3.71 (m, 4H, 1HCH₂ +OCH₃), 3.98 (s, 3H, OCH₃), 4.54 (m, 1HCH₂), 4.89 (broad s, 1H exchangeable, NH), 6.54 (s, 1H Ar), 6.81 (s, 1H Ar), 7.70-7.80 (m, 4H Ar). Mass: (M+H)⁺=335.1.

Synthesis of the iminochloride of type 16.

6,7-dimethoxy-2H-3,1-benzoxazine-2,4(1H)-dione, 13a

2-amino-4,5-dimethoxybenzoic acid (25 g, 0.13 mole) was added to THF (400 ml), then benzyl chloroformate (54 ml, 0.38 mole) was added with very vigorous stirring. The mixture was refluxed overnight, evaporated to dryness and the residue was vacuum evaporated. Ether (425 ml) was poured on the residue, PBr3 (11.88 ml, 0.13 mole) was added and the mixture was refluxed for 48 h. The reaction mixture was filtered and washed with 3×150 ml of ether. The residue was taken up in ether and stirred for 1 h, then filtered, washed and dried. The reaction produced 27 g of the above product in the form of a white powder. Yield: 96%. ¹H NMR (DMSO-d₆, 200 MHz): d 3.82 (s, 3H, OCH₃), 3.88 (s, 3H, OCH₃), 6.66 (s, 1H Ar), 7.27 (s, 1H Ar), 11.58 (s, 1H exchangeable NH).

6,7-dimethoxy-1-methyl-1,2-dihydro-4H-3,1-benzoxazine-2,4-dione, 14a Under an inert atmosphere, 134 mg (3.37 mmoles) of 60% NaH in oil were added to a solution of 500 mg (3.06 mmoles) of 6,7-dimethoxy-1,2-dihydro-4H-3,1-benzoxazine-2,4-dione 13a in 6 ml of anhydrous DMF. After 10 min at room temperature, 219 μl (3.52 mmoles) of Mel were added dropwise. The reaction was left at room temperature for 3 h, then 40 ml of a water-ice mixture were added. The precipitate was filtered and washed with 2×1 ml of EtOH and 3 ml of Et2O. The reaction produced 320 mg of the above product in the form of a white powder. Yield: 59%. ¹H NMR (CDCl3, 300 MHz): d 3.31 (s, 3H, —CH₃), 3.82 (s, 3H, OCH₃), 3.96 (s, 3H, OCH₃), 6.85 (s, 1H Ar), 7.32 (s, 1H Ar).

7,8-dimethoxy-1-methyl-3,4-dihydro-1H-1,4-benzodiazepine-2,5-dione, 15a

A mixture of 320 mg (1.35 mmoles) of 6,7-dimethoxy-1-methyl-1,2-dihydro-4H-3,1-benzoxazine-2,4-dione 14a, 452 mg (3.24 mmoles) of methyl glycinate hydrochloride in 4 ml of pyridine was heated under reflux for 6 h. 3 ml of AcOH were added and the reaction was heated at 130° C. for 12 h. After evaporation to dryness, 10 ml of a water/ice mixture were added. The mixture was allowed to crystallize for 30 minutes at 0° C., then filtered and washed with 2×2 ml of water, 2×1 ml of EtOH and 2×5 ml of Et2O. Recrystallization was in EtOH. The reaction produced 240 mg of the above product as colorless crystals. Yield: 71%. MP: 260-263° C. ¹H NMR (CDCl3, 300 MHz): d 3.42 (s, 3H, NCH₃), 3.75-3.92 (m, 2H, CH₂), 3.98 (s, 6H, 2 ×OCH₃), 6.39 (s, 1H exchangeable, NH), 6.69 (s, 1H Ar), 7.37 (s, 1H Ar).

5-chloro-7,8-dimethoxy-1-methyl-1,3-dihydro-1,4-benzodiazepin-2-one, 16a

A solution of 100 mg (0.40 mmole) of 7,8-dimethoxy-1-methyl-3,4-dihydro-1H-1,4-benzodiazepine-2,5-dione 15a, 280 μl of dimethylaniline, 800 μl of POCl3, in 10 ml of anhydrous CHCl3 was heated in a sealed tube at 125° C. for ¾ hour, then cooled to room temperature. 3 g of silica and 5 ml of CH2Cl2 were added. At 0° C., 1 ml of triethylamine was added. After evaporation to dryness, purification was by chromatography (EtOAc/Hexane 1:1, then EtOAc). The purified fraction was pulverized in 1 ml of Et2O, filtered and washed with 2×2 ml of pentane. The reaction produced 93 mg of the above product in the form of a white powder. Yield: 87%. ¹H NMR (CDCl3, 200 MHz): d 3.42 (s, 3H, NCH₃), 3.77 (broad s, 1H of CH₂), 3.99 (s, 3H, OCH₃), 4.00. (s, 3H, OCH₃), 4.65 (broad s, 1H of CH₂), 6.71 (s. 1H Ar), 7.22 (s, 1H Ar).

Palladium couplings, on the iminochloride of type 17.

5-(3-chlorophenyl)-7,8-dimethoxy-1-methyl-1,3-dihydro-1,4-benzodiazepin-2-one, 17a A mixture of 5-chloro-7,8-dimethoxy-1-methyl-1,3-dihydro-1,4-benzodiazepin-2-one 16a (130 mg, 0.48 mmole), 3-chlorobenzene boronic acid (90.8 mg, 0.58 mmole), K₃PO₄ (118 mg, 0.56 mmole), tetrakis(triphenylphosphine) Pd (0) (15 mg, 0.01 mmole) in 3 ml of DMF was heated at 115° C. for 12 h under an inert atmosphere, then cooled to room temperature. 30 ml of water were added and the mixture was extracted with 3×30 ml of Et₂O. The organic fractions were dried Na₂SO₄, evaporated to dryness, purified by chromatography (EtOAc). Recrystallization was in EtOH. The reaction produced 103 mg of the above product in the form of white crystals. Yield: 62%. MP: 109-111° C. ¹H NMR (CDCl₃, 200 MHz): d 3.41 (s, 3H, NCH₃), 3.77 (s, 3H, OCH₃), 3.99 (s, 3H, OCH₃), 4.34 (AB system, ?d=1.02, J_{AB}=10, 2H, CH₂), 6.67 (s, 1H Ar), 6.79 (s, 1H Ar), 7.19-7.69 (m, 4H Ar).

7,8-dimethoxy-1-methyl-5-(3-pyridyl)-1,3-dihydro-1,4-benzodiazepin-2-one, 17b

By replacing 3-chlorobenzene boronic acid in example 17a by pyridine-3-boronic-1,3-propanediol cyclic acid ester and proceeding in the same manner, the above product was obtained. Yield: 58%. MP: 177-179° C. ¹H NMR (CDCl₃, 200 MHz): d 3.42 (s, 3H, NCH₃), 3.76 (s, 3H, OCH₃), 3.99 (s, 3H, OCH₃), 4.32 (AB system, ?d=1.03, J_{AB}=10, 2H, CH₂), 6.67 (s, 1H Ar), 6.80 (s, 1H Ar), 7.34-7.40 (m, 1H Ar), 8.03-8.09 (m, 1H Ar), 8.68-8.71 (m, 2H Ar).

7,8-dimethoxy-1-methyl-5-(3-nitrophenyl)-1,3-dihydro-1,4-benzodiazepin-2-one, 17c

By replacing 3-chlorobenzene boronic acid in example 17a by 3-nitrobenzene boronic acid and proceeding in the same manner, the above product was obtained. Yield: 43%. MP: 152-155° C. ¹H NMR (CDCl₃, 300 MHz): d-3.43 (s, 3H, NCH₃), 3.76 (s, 3H, OCH₃), 4.01 (s, 3H, OCH₃), 4.35 (AB system, ?d=1.03, J_{AB}=10, 2H, CH₂), 6.64 (s, 1H Ar), 6.82 (s, 1H Ar), 7.59-7.64 (m, 1H Ar), 8.08-8.12 (m, 1H Ar), 8.31-8.35 (m, 1H Ar), 8.49-8.51 (m, 1H Ar).

5-(7,8-dimethoxy-1-methyl-2-oxo-2,3-dihydro-1H-1,4-benzodiazepin-5-yl)-2benzonitrile, 17d

By replacing 3-chlorobenzene boronic acid in example 17a by 3-cyano-4-[(4-methoxybenzyl)oxyl]phenylboronic acid 37c and proceeding in the same manner, the above product was obtained. Yield: 20%. ¹H NMR (DMSO, 300 MHz): d 3.32 (s, 3H, NCH₃), 3.65 (s, 3H, OCH₃), 3.70-3.77 (m, 4H, 1HCH₂+BnOCH₃) 3.90 (s, 3H, OCH₃), 4.51 (m, 1HCH₂), 5.26 (s, 2H, PhCH₂), 6.70 (s, 1H Ar), 6.98 (m, 2HBn), 7.09 (s, 1H Ar), 7.43 (m, 2HBn), 7.55-7.87 (m, 3H Ar).

5-(3-acetylphenyl)-7,8-dimethoxy-1-methyl-1,3-dihydro-1,4-benzodiazepin-2-one, 17e

By replacing 3-chlorobenzene boronic acid in example 17a by 3-acetylbenzene boronic acid and proceeding in the same manner, the above product was obtained. Yield: 43%. MP: 148-150° C. ¹H NMR (CDCl₃, 200 MHz): d 2.64 (s, 3H, CH₃CO), 3.42 (s, 3H, NCH₃), 3.74 (s, 3H, OCH₃), 4.00 (s, 3H, OCH₃), 4.30 (AB system, ?d=1.03, J_{AB}=10, 2H, CH₂), 6.65 (s, 1H Ar), 6.81 (s, 1H Ar), 7.48-8.23 (m, 4H Ar).

5-(4-isoquinolinyl)-7,8-dimethoxy-1-methyl-1,3-dihydro-1,4-benzodiazepin-2-one, 17f

By replacing 3-chlorobenzene boronic acid in example 17a by 2-(isoquinolin-4-yl)-4,4,5,5-tetramethyl-1,3-dioxaborolane and proceeding in the same manner, the above product was obtained. Yield: 34%. MP: 131-135° C. ¹H NMR (CDCl₃, 300 MHz): d 3.53 (s, 3H, NCH₃), 3.55 (s, 3H, OCH₃), 4.00 (s, 3H, OCH₃), 4.50 (AB system, ?d =1,00, J_{AB}=10.5, 2H, CH₂), 6.45 (s, 1H Ar), 6.84 (s, 1H Ar), 7.60-7.69 (m, 2H Ar), 7.89-8.08 (m, 2H Ar), 8.55 (s, 1H Ar), 9.32 (s, 1H Ar).

7,8-dimethoxy-5-(3-hydroxymethylphenyl)-1-methyl-1,3-dihydro-2H-1,4-benzodiazepin-2-one, 17g

By replacing 3-chlorobenzene boronic acid in example 17a by 3-hydroxymethylbenzene boronic acid and proceeding in the same manner, the above product was obtained. Yield: 25%. MP: 143-146° C. ¹H NMR (DMSO, 300 MHz): d 3.44 (s, 3H, NCH₃), 3.63 (s, 3H, OCH₃), 4.01 (s, 3H, OCH₃), 4.35 (AB system, ?d=0.25, J_{AB}=12.8, 2H, CH₂), 4.60 (s, 2H, CH₂OH) 6.69 (s, 1H Ar), 6.91 (s, 1H Ar), 7.61-7.74 (m, 4H Ar).

7,8-dimethoxy-5-(3-hydroxymethylphenyl)-1-methyl-3-propyl-1,3-dihydro-2H-1,4-benzodiazepin-2-one, 17h

21 mg (0.52 mmole) of 60% NaH in oil were added at 0° C. and under an inert atmosphere to a solution of 140 mg (0.47 mmole) of 7,8-dimethoxy-5-(3-hydroxymethylphenyl)-1-methyl-1,3-dihydro-1,4-benzodiazepin-2-one 17g in 5 ml of DMF. The mixture was stirred at room temperature for 1 h. At 0° C., 50 μl of bromopropane were added dropwise. The mixture was stirred overnight at room temperature. 50 ml of H₂O were added and the mixture was extracted wtih 3×50 ml of EtOAc and dried on MgSO₄. The EtOAc was evaporated and the product was purified by chromatography on silica gel (EtOAc/Hexane 1:1 then EtOAc). Recrystallization was in CHCl₃/cHex. Yield: 11%. MP: 134-136° C. ¹H NMR (DMSO, 200 MHz): d 0.85-0.93 (m, 3H, CH₃), 1.35-1.55 (m, 2H, CH₂CH₃), 1.97-2.01 (m, 2H, CHCH₂), 3.31 (s, 3H, NCH₃), 3.42-3.47 (m, 1H, CHCH₂), 3.62 (s, 3H, OCH₃), 3.88 (s, 3H, OCH₃), 4.50 (d, J=5.6, 2H, CH₂OH), 5.21 (t, J=5.6, 1H, OH), 6.66 (s, 1H Ar), 7.08 (s, 1H Ar), 7.38-7.53 (m, 4H Ar). Mass: (M+H)⁺=383.27.

5-(3-aminophenyl)-7,8-dimethoxy-1-methyl-1,3-dihydro-2H-1,4-benzodiazepin-2-one, 17i 7,8-dimethoxy-1-methyl-5-(3-nitrophenyl)-1,3-dihydro-1,4-benzodiazepin-2-one 17c (100 mg, 0.28 mmole), 10 mg of Pd/C (10%), in 2. ml of methanol were stirred. under hydrogen pressure for 8 h, then filtered on cellite, washed with 3×25 ml of methanol, evaporated to dryness and purified by chromatography (EtOAc) to give the above product in the form of white crystals. Yield: 77%. ¹H NMR (CDCl₃, 300 MHz): d 1.60 (s, 2H exchangeable NH₂), 3.40 (s, 3H, NCH₃), 3.78 (s, 3H, OCH₃), 3.99 (s, 3H, OCH₃), 4.38 (AB system, ?d=1.03, J_{AB}=10, 2H, CH₂), 6.76 (m, 3H Ar), 6.89-6.91 (d, 1H Ar), 7.10 (s, 1H Ar), 7.14-7.22 (t, 1H Ar).

5-(3,4-dichlorophenyl)-7,8-dimethoxy-1-methyl-1,3-dihydro-1,4-benzodiazepin-2-one, 17j

By replacing 3-chlorobenzene boronic acid in example 17a by 3,4-dichlorophenyl boronic acid and proceeding in the same manner, the above product was obtained. Yield: 25%. MP: 174-177° C. ¹H NMR (CDCl₃, 200 MHz): d 3.44 (s, 3H, NCH₃), 3.81 (s, 3H, OCH₃), 4.02 (s, 3H, OCH₃), 4.50 (AB system, ?d=1.00, J_{AB}=10.5, 2H, CH₂), 6.67 (s, 1H Ar), 6.82 (s, 1H Ar), 7.52 (s, 2H Ar), 7.83 (s, 1H Ar).

7,8-dimethoxy-1-methyl-5-(3-methylphenyl)-1,3-dihydro-1,4-benzodiazepin-2-one, 17k

By replacing 3-chlorobenzene boronic acid in example 17a by 3-methylbenzene boronic acid and proceeding in the same manner, the above product was obtained. Yield: 20%. MP: 119-121° C. ¹H NMR (CDCl₃, 300 MHz): d 2.39 (s, 3H, CH₃), 3.40 (s, 3H, NCH₃), 3.75 (s, 3H, OCH₃), 3.98 (s, 3H, OCH₃), 4.31 (AB system, ?d=0.99, J_{AB}=10.3, 2H, CH₂), 6.71 (s, 1H Ar), 6.78 (s, 1H Ar), 7.27-7.28 (m, 2H Ar), 7.35-7.38 (m, 1H Ar), 7.53 (s, 1H Ar).

5-(3-formylphenyl)-7,8-dimethoxy-1-methyl-1,3-dihydro-1,4-benzodiazepin-2-one, 171

By replacing 3-chlorobenzene boronic acid in example 17a by 3-formylbenzene boronic acid and proceeding in the same manner, the above product was obtained. Yield: 25%. MP: 175-178° C. ¹H NMR (CDCl₃, 200 MHz): d 3.45 (s, 3H, NCH₃), 3.77 (s, 3H, OCH₃), 4.03 (s, 3H, OCH₃), 4.39 (AB system, ?d=1.01, J_{AB}=10.5, 2H, CH₂), 6.68 (s, 1H Ar), 6.84 (s, 1H Ar), 7.60-7.67 (m, 1H Ar), 8.01-8.05 (m, 2H Ar), 8.18 (s, 1H Ar), 10.09 (s, 1H, CHO).

5-[3-(benzylaminomethyl)phenyl]-7,8-dimethoxy-1-methyl-1,3-dihydro-2H-1,4-benzodiazepin-2-one hydrochloride, 17m

A mixture of 200 mg (0.53 mmole) of 5-(3-formylphenyl)-7,8-dimethoxy-1-methyl-1,3-dihydro-1,4-benzodiazepin-2-one 17IIy, 60 μl (0.56 mmole) of benzylamine in 5 ml of MeOH was heated overnight under reflux. At 0° C., 53 mg (1.4 mmoles) of NaBH₄ was added in small portions. The reaction was stirred at room temperature for 1 h. The methanol was evaporated. 30 ml of H₂O were added and the mixture was extracted with 3×50 ml of EtOAc. 10 ml of CH₂C₁₂ were added and HCl was bubbled in at 0° C. until the solution became saturated. The solvent was evaporated and the residue was triturated in Et₂O, discarding the supernatant several times. Recrystallization was in a mixture of EtOH/Et₂O. The crystals were filtered and dried. Yield: 75%. MP: 88-90° C. ¹H NMR (DMSO, 200 MHz): d 3.41 (s, 3H, NCH₃), 3.50-3.69 (m, 4H, 1HCH₂ +OCH₃), 3.98 (s, 3H, OCH₃), 4.13-4.23 (m, 4H, 2CH₂Ph), 4.47-4.54 (m, 1H, 1HCH₂), 6.70 (s, 1H Ar), 7.23 (s, 1H Ar), 7.38-7.98 (m, 9H Ar). Mass: (M+H)⁺⁺=430.18.

N-[3-(7,8-dimethoxy-1-methyl-2-oxo-2,3-dihydro-1H-1,4-benzodiazepin-5-yl) phenyl]acetamide, 17n

By replacing 3-chlorobenzene boronic acid in example 17a by 3-acetamidobenzene boronic acid and proceeding in the same manner, the above product was obtained. Yield: 15%. ¹H NMR (DMSO, 300 MHz): d 2.01 (s, 3H, CH₃CO), 3.31 (s, 3H, NCH₃), 3.66 (s, 3H, OCH₃), 3.90 (s, 3H, OCH₃), 4.12 (AB system, ?d=0.78, J_{AB}=10.17, 2H, CH₂), 6.71 (s, 1H Ar), 7.08 (s, 1H Ar), 7.29-7.78 (m, 4H Ar), 10.01 (s, 1H, NHAc).

7,8-dimethoxy-1-methyl-5-(3,5-methylenedioxyphenyl)-1,3-dihydro-2H-1,4-benzodiazepin-2-one, 17o

By replacing 3-chlorobenzene boronic acid in example 17a by 3,5-methylenedioxybenzene boronic acid and proceeding in the same manner, the above product was obtained. Yield: 55%. MP: 161-162° C. 1H NMR (CDCl₃, 300 MHz): d 3.39 (s, 3H, NCH₃), 3.74-3.79 (m, 4H, 1HCH₂+OCH₃), 3.98 (s, 3H, OCH₃), 4.72-4.76 (m, 1HCH₂), 6.03 (s, 2H, CH₂O₂), 6.75-7.28 (m, 5H Ar).

Regioselective halogenation and synthesis of benzodiazepines of type 24

Tert-butyl(2-methoxy-5-nitrophenoxy)diphenylsilane, 18a

At 0° C. under an inert atmosphere, a solution of 7 g (41 mmoles) of 3-hydroxy-4-methoxynitrobenzene dissolved in 50 ml of DMF was added to a solution of 2 g (50 mmoles) of sodium hydride dissolved in 50 ml of DMF. After 30 minutes at room temperature, 12.9 ml (50 mmoles) of tert-butylchlorodiphenyl silane were added dropwise at 0C. The reaction was stirred at room temperature for 12 hours, then diluted with 10 volumes of water and extracted with 3×50 ml of Et₂O. The organic phase was washed with 100 ml of 1 M HCl and NaCl (sat), dried on Na₂SO₄, filtered and evaporated to dryness to give the above product. Yield: 82%. ¹H NMR (CDCl₃, 200 MHz): d 1.18 (s, 9H, C(CH₃)₃), 3.61 (s, 3H, OCH₃), 6.74-6.78 (d, 1H Ar), 7.34-7.82 (m, 12H Ar).

Tert-butyl(2-methoxy-4-nitrophenoxy)diphenylsilane, 18b

At 0° C. under an inert atmosphere, a solution of 1 g (6.45 mmoles) of 4-nitrocatechol dissolved in 5 ml of DMF was added to a solution of 480 mg (7.10 mmoles) of imidazole dissolved in 5 ml of DMF. After 30 minutes at room temperature, 1.77 ml (6.77 mmoles) of tert-butylchlorodiphenyl silane were added dropwise at 0° C. The reaction was stirred at room temperature for 12 hours, then diluted with 10 volumes of water and extracted with 3×50 ml of Et₂O. The organic phase was washed with 100 ml of 1 M HCl and NaCl (sat), dried on Na₂SO₄, filtered, and evaporated to dryness to yield a brown oil. Said oil was dissolved in 30 ml of DMF, then 2.86 g (20.69 mmoles) of K₂CO₃ were added and the mixture was stirred for 30 minutes. lodomethane (1.37 ml, 22.04 mmoles) was added and the reaction left for 2 hours, then diluted with 10 volumes of water and extracted with 3×100 ml of Et₂O. The organic phase was. washed with 100 ml of 10% NaOH, dried on Na₂SO₄, filtered, evaporated to dryness and purified by chromatography on silica gel (hexane/CH₂Cl₂, 2:1 ). Yield: 57%. ¹H NMR (CDCl₃, 200 MHz): d 1.15 (s, 9H, C(CH₃)₃), 3.58 (s, 3H, OCH₃), 6.71-6.76 (d, 1H Ar), 7.36-7.82 (m, 12H Ar).

3-{[tert-butyl(diphenyl)silyl]oxy}-4-methoxyaniline, 19a

By replacing 7,8-dimethoxy-5-(3 ′-hex-1-ynylphenyl)-1-N-methyl-1,3-dihydro-2H-1,4-benzodiazepin-2-one 6b in example 6e by tert-butyl(2-methoxy-5-nitrophenoxy)diphenylsilane 18a and proceeding in the same manner, the above product was obtained. Yield: 98%. ¹H NMR (CDCl₃, 300 MHz): d 1.12 (s, 9H, C(CH₃)₃), 3.59 (s, 3H, OCH₃), 6.23 (s, 1H Ar), 6.24-6.30 (d, 1H Ar), 6.62-6.65 (d, 1H Ar), 7.28-7.75 (m, 10H Ar).

4-{[tert-butyl(diphenyl)silyl]oxy}-3-methoxyaniline, 19b

By replacing 7,8-dimethoxy-5-(3 ′-hex-1-ynylphenyl)-1-N-methyl-1,3-dihydro-2H-1,4-benzodiazepin-2-one 6b in example 6e by tert-butyl(2-methoxy-4-nitrophenoxy)diphenylsilane 18b and proceeding in the same manner, the above product was obtained. Yield: 95%. ¹H NMR (CDCl₃, 200 MHz): d 1.08 (s, 9H, C(CH₃)₃), 3.53 (s, 3H, OCH₃), 5.98-6.02 (m, 1H Ar), 6.18 (s, 1H Ar), 6.50-6.54 (m, 1H Ar), 7.37-7.72 (m, 10H Ar).

3-(2-amino-5-{[tert-butyl(diphenyl)silyl]oxy}-4-methoxybenzoyl)benzonitrile, 20b

At 0° C. under an inert atmosphere, 3.5 g (9.22 mmoles) of 4-{[tert-butyl(diphenyl)silyl]oxy}-3-methoxyaniline 19b dissolved in 10 ml of 1,2-dichloroethane, 2.35 g (18.36 mmoles) of isophthalonitrile, and 1.34 g (10.00 mmoles) of AlCl₃ were added to a solution of 10 ml of boron tribromide (1M/CH₂Cl₂, 10 mmoles) and stirred at room temperature for 30 minutes. The dichloromethane was evaporated. The mixture was heated under reflux for 12 hours, then cooled. 10 ml of 1 M HCl were added at 0° C. and the mixture was stirred at 75° C. for 1 hour. After adding 50 ml of water, the mixture was extracted with 3×100 ml of CH₂Cl₂. The organic fractions were dried on Na₂SO₄, filtered, evaporated to dryness and purified by chromatography on silica gel (EtOAc/hexane, 1:3 ). Yield: 30%. ¹H NMR (CDCl₃, 300 MHz): d 1.06 (s, 9H, C(CH₃)₃), 3.77 (s, 3H, OCH₃), 6.09 (s, 1H Ar), 6.17 (broad s, 2H, NH₂), 6.47 (s, 1H Ar), 7.37-7.86 (m, 14H Ar).

3-(2-amino-4-{[tert-butyl(diphenyl)silyl]oxy}-5-methoxybenzoyl)benzonitrile, 20a

By replacing 4-{[tert-butyl(diphenyl)silyl]oxy}-3-methoxyaniline 19b in example 20b by 3-{[tert-butyl(diphenyl)silyl]oxy}-4-methoxyaniline 19a and proceeding in the same manner, the above product was obtained. Yield: 38%. ¹H NMR (CDCl₃, 300 MHz): d 1.11 (s, 9H, C(CH₃)₃), 3.41 (s, 3H, OCH₃), 5.90 (broad s, 2H, NH₂), 6.01 (s, 1H Ar), 6.67 (s, 1H Ar), 7.39-7.96 (m, 14H Ar).

2-bromo-N-[5-{[tert-butyl(diphenyl)silyl]oxy}-2-(3-cyanobenzoyl)-4-methoxyphenyl]acetamide, 21b

To a solution of 3-(2-amino-5-{[tert-butyl(diphenyl)silyl]oxy}-4-methoxybenzoyl)benzonitrile 20b (0.4 g, 0.79 mmole) in 5 ml of dichloromethane at 0-5° C., bromoacetate bromide (82 ??, 0.94 mmole) was added and then 10% Na₂CO₃ (2.4 ml) was added dropwise. The reaction was stirred at this temperature for 1 hour. The two phases were separated and the organic phase was washed with 10 ml of water, dried on Na₂SO₄, filtered and evaporated to dryness (455 mg). Yield: 92%. ¹H NMR (CDCl₃, 200 MHz): d 1.15 (s, 9H, C(CH₃)₃), 3.29 (s, 3H, OCH₃), 3.95 (s, 2H; CH₂), 6.75 (s, 1H Ar), 7.37-7.74 (m, 14H Ar), 8.23 (s, 1H Ar), 11.55 (broad s, 1H, NH).

2-bromo-N-[4-{[tert-butyl(diphenyl)silyl]oxy}-2-(3-cyanobenzoyl)-5-methoxyphenyllacetamide, 21a

By replacing 3-(2-amino-5-{[tert-butyl(diphenyl)silyl]oxy}-4-methoxybenzoyl)benzonitrile 20b in example 21b by 3-(2-amino-4-{[tert-turyl(diphenyl)silyl]oxy}-5-methoxybenzoyl)benzonitrile 20a and proceeding in the same manner, the above product was obtained. Yield: 90%. ¹H NMR (CDCl₃, 300 MHz): d 1.14 (s, 9H, C(CH₃)₃), 3.28 (s, 3H, OCH₃), 3.94 (s, 2H; CH₂), 6.74 (s, 1H Ar), 7.37-7.87 (m, 14H Ar), 8.23 (s, 1H Ar), 11.52 (broad s, 1H, NH).

3-(7-hydroxy-8-methoxy-2-oxo-2,3-dihydro-1H-1,4-benzodiazepin-5-yl)benzonitrile, 22b

2-bromo-N-[5-{[tert-butyl(diphenyl)silyl]oxy}-2-(3-cyanobenzoyl)-4-methoxyphenyl]acetamide 21b (0.5 g, 0.79 mmole) in solution in NH3 (7N)/MeOH (10 ml) was stirred in a CaCl₂ tube for 30 minutes at 0° C. then for 30 minutes at room temperature. It was then heated under reflux for 2 hours, evaporated to dryness and purified by chromatography on silica gel (MeOH/CH₂Cl₂, 1:9). Yield: 95%. ¹H NMR (DMSO-d₆, 300 MHz): d 3.63 (s, 3H, OCH₃), 4.12 (broad s, 2H; CH₂), 6.65 (s, 1H Ar), 6.72 (s, 1H Ar), 7.65-7.99 (m, 4H Ar), 10.14 (broad s, 1H, NH), 10.33 (broad s, 1H, OH).

3-(8-hydroxy-7-methoxy-2-oxo-2,3-dihydro-1H-1,4-benzodiazepin-5-yl)benzonitrile, 22a

By replacing 2-bromo-N-[5-{[tert-butyl(diphenyl)silyl]oxy}-2-(3-cyanobenzoyl)-4-methoxyphenyl]acetamide 21b in example 22b by 2-bromo-N-[4-{[tert-butyl(diphenyl)silyl]oxy}-2-(3-cyanobenzoyl)-5-methoxyphenyl]acetamide 21a and proceeding in the same manner, the above product was obtained. Yield: 30%. 1H NMR (DMSO-d₆, 200 MHz): d 3.64 (s, 3H, OCH₃), 4.13 (broad s, 2H; CH₂), 6.66 (s, 1H Ar), 6.72 (s, 1H Ar), 7.61-2.25 (m, 4H Ar), 10.13 (broad s, 1H, NH), 10.33 (broad s, 1H, OH).

3-(6-bromo-7-hydroxy-8-methoxy-2-oxo-2,3-dihydro-1H-1,4-benzodiazepin-5-yl)benzonitrile, 23b

A mixture of 3-(7-hydroxy-8-methoxy-2-oxo-2,3-dihydro-1H-1,4-benzodiazepin-5-yl)benzonitrile 22b (150 mg, 0.49 mmole) and N-bromosuccinimide (90 mg, 0.51 mmole) in acetic acid was heated at 60° C. for 2 hours, evaporated to dryness and crystallized in Et₂O. Yield: 70%. ¹H NMR (DMSO-d₆, 300 MHz): d 3.92 (s, 3H, OCH₃), 4.20 (AB system, ?d=0.6, J_{AB}=10 Hz, 2H, NCH₂), 6.91 (s, 1H Ar), 7.61-7.93 (m, 4H Ar +OH), 10.36 (broad s, 1H, NH).

3-(9-iodo-8-hydroxy-7-methoxy-2-oxo-2,3-dihydro-1H-1,4-benzodiazepin-5-yl)benzonitrile, 23a

By replacing 3-(7-hydroxy-8-methoxy-2-oxo-2,3-dihydro-1H-1,4-benzodiazepin-5-yl)benzonitrile 22b in example 23b by 3-(8-hydroxy-7-methoxy-2-oxo-2,3-dihydro-1H-1,4-benzodiazepin-5-yl)benzonitrile 22a and N-bromosuccinimide by N-iodosuccinimide and proceeding in the same manner, the above product was obtained. Yield: 83%. ¹H NMR (DMSO-d₆, 200 MHz): d 3.72 (s, 3H, OCH₃), 4.08 (m 2H, NCH₂), 6.78 (s, 1H Ar), 7.61-7.93 (m, 4H Ar ), 9.22 (broad s, 1H, OH), 10.61 (broad s, 1H, NH).

3-(6-iodo-7-hydroxy-8-methoxy-2-oxo-2,3-dihydro-1H-1,4-benzodiazepin-5-yl)benzonitrile, 23c

By replacing N-bromosuccinimide in example 23b by N-iodosuccinimide and proceeding in the same manner, the above product was obtained. Yield: 65%. ¹H NMR (DMSO-d₆, 300 MHz): d 3.72 (s, 3H, OCH₃), 4.11 (m, 2H, NCH₂), 6.77 (s, 1H Ar), 7.65-8.03(m, 4H Ar), 9.27 (broad s, 1H, OH), 11.13 (broad s, 1H, NH).

3-(9-bromo-8-hydroxy-7-methoxy-2-oxo-2,3-dihydro-1H-1,4-benzodiazepin-5-yl)benzonitrile, 23d

By replacing 3-(7-hydroxy-8-methoxy-2-oxo-2,3-dihydro-1H-1,4-benzodiazepin-5-yl)benzonitrile 22b in example 23b by 3-(8-hydroxy-7-methoxy-2-oxo-2,3-dihydro-1H-1,4-benzodiazepin-5-yl)benzonitrile 22a and proceeding in the same manner, the above product was obtained. Yield: 86%. ¹H NMR (DMSO-d₆, 200 MHz): d 3.92 (s, 3H, OCH₃), 4.18 (m 2H, NCH₂), 6.84 (s, 1H Ar), 7.61-7.93 (m, 4H Ar ), 9.22 (broad s, 1H, OH), 11.05 (broad s, 1H, NH).

3-(6-bromo-7,8-dimethoxy-1-methyl-2-oxo-2,3-dihydro-1H-1,4-benzodiazepin-5-yl)benzonitrile, 24b

3-(6-bromo-7-hydroxy-8-methoxy-2-oxo-2,3-dihydro-1H-1,4-benzodiazepin-5-yl)benzonitrile 23b (120 mg, 0.31 mmole) was dissolved in 2 ml of DMF, then 131 mg (0.95 mmole) of K₂CO₃ were added and the reaction was stirred for 30 minutes. Iodomethane (44 □L, 0.71 mmole) was added and allowed to react for 6 hours. A water/ice mixture was added and the precipitate was filtered. Yield: 70%. ¹H NMR (CDCl₃, 300 MHz): d 3.39 (s, 3H, NCH₃), 3.88 (s, 3H, OCH₃), 4.00 (s, 3H, OCH₃), 4.30 (AB system, ?d=0.9, J_{AB}=10 Hz, 2H, NCH₂), 6.84 (s, 1H Ar), 7.49-7.51 (t, 1H Ar), 7.67-7.70 (d, 1H Ar), 7.74 (s, 1H Ar), 7.81-7.83 (d, 1H Ar).

3-(9-iodo-7,8-dimethoxy-1-methyl-2-oxo-2,3-dihydro-1H-1,4-benzodiazepin-5-yl)benzonitrile, 24a

By replacing 3-(6-bromo-7-hydroxy-8-methoxy-2-oxo-2,3-dihydro-1H-1,4-benzodiazepin-5-yl)benzonitrile 23b in example 24b by 3-(9-iodoo-8-hydroxy-7-methoxy-2-oxo-2,3-dihydro-1H-1,4-benzodiazepin-5-yl)benzonitrile 23a and proceeding in the same manner, the above product was obtained. Yield: 91%. ¹H NMR (CDCl₃, 200 MHz): d 3.31 (s, 3H, NCH₃), 3.77 (s, 3H, OCH₃), 3.97 (s, 3H, OCH₃), 4.31 (AB system, ?d=1.0, J_{AB}=10 Hz, 2H, NCH₂), 6.67 (s, 1H Ar), 7.53-8.03 (m, 4H Ar).

3-(6-iodo-7,8-dimethoxy-1-methyl-2-oxo-2,3-dihydro-1H-1,4-benzodiazepin-5-yl)benzonitrile, 24c

By replacing 3-(6-bromo-7-hydroxy-8-methoxy-2-oxo-2,3-dihydro-1H-1,4-benzodiazepin-5-yl)benzonitrile 23b in example 24b by 3-(6-iodoo-7-hydroxy-8-methoxy-2-oxo-2,3-dihydro-1H-1,4-benzodiazepin-5-yl)benzonitrile 23c and proceeding in the same manner, the above product was obtained. Yield: 58%. ¹H NMR (CDCl₃, 200 MHz): d 3.31 (s, 3H, NCH₃), 3.78 (s, 3H, OCH₃), 3.96 (s, 3H, OCH₃), 4.28 (AB system, ?d=1.0, J_{AB}=10 Hz, 2H, NCH₂), 6.67 (s, 1H Ar), 7.53-8.03 (m, 4H Ar).

3-(9-bromo-7,8-dimethoxy-1-methyl-2-oxo-2,3-dihydro-1H-1,4-benzodiazepin-5-yl)benzonitrile, 24d

By replacing 3-(6-bromo-7-hydroxy-8-methoxy-2-oxo-2,3-dihydro-1H-1,4-benzodiazepin-5-yl)benzonitrile 23b in example 24b by 3-(9-bromo-7-hydroxy-8-methoxy-2-oxo-2,3-dihydro-1H-1,4-benzodiazepin-5-yl)benzonitrile 23d and proceeding in the same manner, the above product was obtained. Yield: 70%. ¹H NMR (CDCl₃, 300 MHz): d 3.31 (s, 3H, NCH₃), 3.79 (s, 3H, OCH₃), 3.99 (s, 3H, OCH₃),4.30 (AB system, ?d=1.0, J_{AB}=10 Hz, 2H, NCH₂), 6.65 (s, 1H Ar), 7.57-8.02 (m, 4H Ar).

Palladium couplings, on benzodiazepinones of type 25.

3-(7,8-dimethoxy-1-methyl-2-oxo-6-phenyl-2,3-dihydro-1H-1,4-benzodiazepin-5-yl)benzonitrile, 25b

A mixture of 67 mg (0.162 mmole) of 3-(6-bromo-7,8-dimethoxy-1-methyl-2-oxo-2,3-dihydro-1H-1,4-benzodiazepin-5-yl)benzonitrile 24b, 39 mg (0.32 mmole) of benzene boronic acid, 63 mg (0.30 mmole) of K₃PO₄, 19 mg (0.016 mmole) of tetrakis(triphenylphosphine) Pd(0) in 1 ml of DMF was heated at 115° C. for 16 hours, then cooled to room temperature, diluted with 10 volumes of water and extracted with 3×100 ml of Et₂O. The organic phase was dried on Na₂SO₄, filtered, evaporated to dryness and purified by chromatography on silica gel (CH₂Cl₂/Et₂O, 1:1). Yield: 40%. ¹H NMR (CDCl₃, 200 MHz): d 3.45 (s, 3H, NCH₃), 3.50 (s, 3H, OCH₃), 4.03 (s, 3H, OCH₃),4.50 (AB system, ?d=0.8, J_{AB}=10 Hz, 2H, NCH₂), 6.87-7.51 (m, 7H Ar), 7.33-7.80 (m, 3H Ar).

3-(7,8-dimethoxy-1-methyl-2-oxo-9-phenyl-2,3-dihydro-1H-1,4-benzodiazepin-5-yl)benzonitrile, 25a

By replacing 3-(6-bromo-7,8-dimethoxy-1-methyl-2-oxo-2,3-dihydro-1H-1,4-benzodiazepin-5-yl)benzonitrile 24b in example 25b by 3-(9-iodo-7,8-dimethoxy-1-methyl-2-oxo-2,3-dihydro-1H-1,4-benzodiazepin-5-yl)benzonitrile 24d and proceeding in the same manner, the above product was obtained. Yield: 51%. ¹H NMR (CDCl₃, 300 MHz): d 2.44 (s, 3H, NCH₃), 3.63 (s, 3H, OCH₃), 3.80 (s, 3H, OCH₃), 4.45 (AB system, ?d=1.0, J_{AB}=10 Hz, 2H, NCH₂), 6.69 (s, 1H Ar), 7.41-8.27 (m, 9H Ar).

Tert-butyl-3-[5-(cyanophenyl)-(7,8-dimethoxy-1-methyl-2-oxo-2,3-dihydro-1H-1,4-benzodiazepin-9-yl)phenyl]prop-2-ynylcarbamate, 25c

By replacing 5-(3-bromophenyl)-7,8-dimethoxy-1-methyl-1,3-dihydro-2H-1,4-benzodiazepin-2-one 4b in example 6a by 3-(9-iodo-7,8-dimethoxy-1-methyl-2-oxo-2,3-dihydro-1H-1,4-benzodiazepin-5-yl)benzonitrile 24a and proceeding in the same manner, the above product was obtained. Yield: 55%. ¹H NMR (CDCl₃, 200 MHz): d 1.48 (s, 9H, C(CH₃)₃), 3.36 (s, 3H, NCH₃), 3.75 (s, 3H, OCH₃), 4.03 (s, 3H, OCH₃), 4.23-4.26 (d, 2H, NHCH₂), 4.32 (AB system, ?d=1.0, J_{AB}=10 Hz, 2H, CH₂), 6.61 (s, 1H Ar), 7.51-7.99 (m, 4H Ar).

Methyl (2E)-3-[5-(cyanophenyl)-7,8-dimethoxy-1-methyl-2-oxo-2,3-dihydro-1H-1,4-benzodiazepin-9-yl)phenyl]acrylate, 25d

In a tube sealed under argon, 92 mg (0.2 mmole) of 3-(9-iodo-7,8-dimethoxy-1-methyl-2-oxo-2,3-dihydro-1H-1,4-benzodiazepin-5-yl)benzonitrile 24a was dissolved in 0.5 ml of DMF. One mg (4.4 μmoles) of palladium acetate, 23 □l (0.25 mmole) of methyl acrylate and 53 μl (0.22 mmole) of tributylamine were added. The solution was placed in a microwave at 200 watts for 10 minutes. Water was added, the mixture was extracted with EtOAc, dried on Na₂SO₄, evaporated to dryness, and purified by chromatography on silica gel (CH₂Cl₂/EtOAc, 2:8). Yield: 27%. ¹H NMR (CDCl₃, 200 MHz): d 3.13 (s, 3H, NCH₃), 3.78 (s, 3H, OCH₃), 3.84 (s, 3H, OCH₃), 3.93 (s, 3H, OCH₃), 4.37 (AB system, ?d=1.0, J_{AB}=10 Hz, 2H, CH₂), 6.65-6.73 (m, 2H Ar), 7.51-7.99 (m, 5H Ar).

Tert-butyl-3-[S-(cyanophenyl)-(7,8-dimethoxy-1-methyl-2-oxo-2,3-dihydro-1H-1,4-benzodiazepin-6-yl)phenyl]prop-2-ynylcarbamate, 25e

By replacing 5-(3-bromophenyl)-7,8-dimethoxy-1-methyl-1,3-dihydro-2H-1,4-benzodiazepin-2-one 4b in example 6a by 3-(6-iodo-7,8-dimethoxy-1-methyl-2-oxo-2,3-dihydro-1H-1,4-benzodiazepin-5-yl)benzonitrile 24c and proceeding in the same manner, the above product was obtained. Yield: 59%. ¹H NMR (CDCl₃, 200 MHz): d 1.48 (s, 9H, C(CH₃)₃), 3.36 (s, 3H, NCH₃), 3.75 (s, 3H, OCH₃), 4.02 (s, 3H, OCH₃), 4.23-4.26 (d, 2H, NHCH₂), 4.30 (AB system, ?d=1.0, J_{AB}=10 Hz, 2H, CH₂), 6.61 (s, 1H Ar), 7.55-8.01 (m, 4H Ar).

[9-(3-aminoethynyl)-7,8-dimethoxy-1-methyl-2-oxo-2,3-dihydro-1H-1,4-benzodiazepin-5-yl]benzonitrile, 25f

A mixture of 25 mg (0.05 rnmole) of tert-butyl 3-[5-(cyanophenyl)-(7,8-dimethoxy-1-methyl-2-oxo-2,3-dihydro-1H-1,4-benzodiazepin-9-yl)phenyl]prop-2-ynylcarbamate 25c, (40 □L, 0.52 mmole) of trifluoroacetic acid and 2 ml of CH₂Cl₂ was stirred under an inert atmosphere at room temperature for 2 hours, then evaporated to dryness. The product was crystallized in EtOAc/hexane. Yield: 97%. ¹H NMR (DMSO-d₆, 200 MHz): d 3.29 (s, 3H, NCH₃), 3.74 (s, 3H, OCH₃), 3.95 (s, 3H, OCH₃), 4.17 (m, 2H, CH₂), 4.26 (AB system, ?d=0.7, J_{AB}=11 Hz, 2H, NCH₂), 6.90 (s, 1H Ar), 7.66-8.07 (m, 4H Ar), 8.41 (broad s, 3H, NH₂).

[6-(3-aminoethynyl)-7,8-dimethoxy-1-methyl-2-oxo-2,3-dihydro-1H-1,4-benzodiazepin-5-yl]benzonitrile, 25g

By replacing tert-butyl-3-[5-(cyanophenyl)-(7,8-dimethoxy-1-methyl-2-oxo-2,3-dihydro-1H-1,4-benzodiazepin-9-yl)phenyl]prop-2-ynylcarbamate 25c in example 25f by tert-butyl-3-[5-(cyanophenyl)-(7,8-dimethoxy-1-methyl-2-oxo-2,3-dihydro-1H-1,4-benzodiazepin-6-yl)phenyl]prop-2-ynylcarbamate 25e and proceeding in the same manner, the above product was obtained. Yield: 96%. ¹H NMR (DMSO-d₆, 200 MHz): d 3.33 (s, 3H, NCH₃), 3.75 (s, 3H, OCH₃), 4.01(s, 3H, OCH₃), 4.20 (m, 2H, CH₂), 4.25 (AB system, ?d=0.7, J_{AB}=11 Hz, 2H, NCH₂), 6.63 (s, 1H Ar), 7.53-7.99 (m, 4H Ar), 8.52 (broad s, 3H, NH₂).

Synthesis of benzodiazepinones of type 29

4-bromo-3,5-dimethoxyaniline, 26a

3.06 g (20 mmoles) of 3,5-dimethoxyaniline were dissolved in 50 ml of CH₂C1₂. The mixture was cooled to −10° C. and 8.19 g (20 mmoles) of 2,4,4,6-tetrabromocyclohexa-2,5-dienone were added one spatula at a time, without allowing the temperature to rise above −5° C. The reaction was then returned to room temperature and stirred for 3 hours, evaporated to dryness, triturated in ether, filtered and washed with ether. The reaction produced 3.20 g (13.8 mmoles) of the above product. Yield: 69%. ¹H NMR (CDCl₃, 300 MHz): d 3.74 (s, 2H, NH₂), 3.84 (s, 6H, 2 x OCH₃), 5.96 (s, 2H Ar).

3-(2-amino4-methoxybenzoyl)benzonitrile, 27a

By replacing 3,4-dimethoxyaniline in example 2a by 3-methoxyaniline and proceeding in the same manner, the above product was obtained. Yield: 43%. ¹H NMR (CDCl₃, 300 MHz): d 3.82 (s, 3H, OCH₃), 6.17 (s, 1H Ar), 6.46 (s, 2H exchangeable NH₂), 6.21-6.24 (d, 1H Ar), 7.54-7.59 (m, 1H Ar), 7.75-7.95 (m, 5H Ar).

3-(2-amino-5-methoxybenzoyl)benzonitrile, 27b

By replacing 3,4-dimethoxyaniline in example 2a by 4-methoxyaniline and proceeding in the same manner, the above product was obtained. Yield: 48%. ¹H NMR (CDCl₃, 200 MHz): d 3.70 (s, 3H, OCH₃), 5.88 (s, 2H exchangeable NH₂), 6.75-6.84 (m, 2H Ar), 7.05-7.11 (m, 1H Ar), 7.59-7.73 (m, 2H Ar), 7.83-7.99 (m, 2H Ar).

3-(2-amino-6-methoxybenzoyl)benzonitrile, 27c

This product was obtained at the same time as 3-(2-amino-4-methoxybenzoyl)benzonitrile 27a. Yield: 22%. ¹H NMR (CDCl₃, 300 MHz): d 3.49 (s, 3H, OCH₃), 5.05 (s, 2H exchangeable NH₂), 6.23-6.25 (d, 1H Ar), 6.38-6.42 (d, 1H Ar), 7.20-7.28 (m, 1H Ar), 7.50-7.55 (m, 2H Ar), 7.74-7.77 (m, 1H Ar), 7.90-7.96 (m, 1H Ar).

(2-amino-4-methoxyphenyl)(phenyl)methanone, 27d

By replacing isophthalonitrile in example 2a by benzonitrile, and 3,4-dimethoxyaniline by 3-methoxyaniline, and proceeding in the same manner, the above product was obtained. Yield: 68%. ¹H NMR (CDCl₃, 300 MHz): d 3.82 (s, 3H, OCH₃), 6.15-6.19 (m, 2H Ar), 6.36 (s, 2H exchangeable NH₂), 7.38-7.50 (m, 4H Ar), 7.57-7.62 (m, 2H Ar).

(2-amino-6-methoxyphenyl)(phenyl)methanone, 27e

This product was obtained at the same time as (2-amino-4-methoxyphenyl)(phenyl)methanone 27d. Yield: 16%. ¹H NMR (CDCl₃, 300 MHz): d 3.53 (s, 3H, OCH₃), 4.61 (s, 2H exchangeable NH₂), 6.27-6.30 (d, 1H Ar), 6.37-6.40 (d, 1H Ar), 7.16-7.22 (t, 1H Ar), 7.38-7.43 (m, 2H Ar), 7.49-7.53 (m, 1H Ar), 7.74-7.77 (m, 2H Ar).

(2-amino-3-bromo-4.5-dimethoxyphenyl)(phenyl)methanone, 27f

15 g of 40% HBr by weight in water were added dropwise at 0° C. to a solution of 900 mg (3.5 mmoles) of 2-amino-4,5-dimethoxybenzophenone 2c in 60 ml of DMSO. The reaction was heated at 60° C. for 24 hours. 400 ml of water were added and the mixture was extracted with 4×200 ml of EtOAc and dried on MgSO₄. The EtOAc was evaporated and the product was purified by chromatography on silica gel (EtOAc/hexane, 1:4). Yield: 65%. ¹H NMR (CDCl₃, 300 MHz): d 3.67 (s, 3H, OCH₃), 3.97 (s, 3H, OCH₃), 6.59 (s, 2H, NH₂), 7.06 (s, 1H Ar), 7.47-7.65 (m, 5H Ar). Mass: (M +H)⁺=335.98+337.98.

3-(2-amino-4,6-dimethoxybenzoyl)benzonitrile, 27g

By replacing 3,4-dimethoxyaniline in example 2a by 3,5-dimethoxyaniline and proceeding in the same manner, the above product was obtained. Yield: 55%. ¹H NMR (CDCl₃, 300 MHz): d 3.39 (s, 3H, OCH₃), 3.78 (s, 3H, OCH₃), 5.87(s, 1H Ar), 6.08 (s, 1H Ar), 6.36 (s, 2H exchangeable NH₂), 7.61-7.97 (m, 4H Ar).

3-(6-amino-3-bromo-2,4-dimethoxybenzoyl)benzonitrile, 27h

By replacing 3,4-dimethoxyaniline in example 2a by 4-bromo-3,5-dimethoxyaniline 26a and proceeding in the same manner, the above product was obtained. Yield: 59%. ¹H NMR (CDCl₃, 200 MHz): d 3.49 (s, 3H, OCH₃), 3.97 (s, 3H, OCH₃), 5.89 (s, 1H Ar), 6.09 (s, 2H exchangeable NH₂), 7.51-7.96 (m, 4H Ar).

3-(8-methoxy-2-oxo-2,3-dihydro-1H-1,4-benzodiazepin-5-yl)benzonitrile, 28a

A mixture of 3-(2-amino-4-methoxybenzoyl)benzonitrile 27a (4 g, 15.9 mmoles), ethyl glycinate.HCl (4 g, 28.6 mmoles), and 40 ml of anhydrous pyridine was heated under reflux in an inert atmosphere for 36 hours. Two 2 g fractions (14.3 mmoles) of ethyl glycinate.HCl, were added every 10 hours. After returning to room temperature, the mixture was evaporated to dryness. 200 ml of water were added and the mixture was extracted with 3×200 ml of dichloromethane. The organic phases were dried on Na₂SO₄, purified by chromatography (EtOAc) and recrystallinzed in EtOH/EtO₂ to give the above product in the form of colorless crystals. Yield: 18%. ¹H NMR (CDCl₃, 300 MHz): d 3.90 (s, 3H, OCH₃), 4.35 (s 2H, CH₂), 6.65 (m, 1H Ar), 6.73-6.77 (m, 1H Ar), 7.15-7.18 (d, 1H Ar), 7.48-7.54 (t, 1H Ar), 7.72-7.75 (m, 1H Ar), 7.80-7.84 (m, 1H Ar), 7.86 (m, 1H Ar), 9.08 (s, 1H exchangeable, —NH).

3-(6-methoxy-2-oxo-2,3-dihydro-1H-1,4-benzodiazepin-5-yl)benzonitrile, 28b

By replacing 3-(2-amino-4-methoxybenzoyl)benzonitrile 27a in example 28a by 3-(2-amino-6-methoxybenzoyl)benzonitrile 27c and proceeding in the same manner, the above product was obtained. Yield: 12%. ¹H NMR (CDCl₃, 300 MHz): d 3.53 (s, 3H, OCH₃), 4.37 (AB system, ?d=0.87, J_{AB}=11 Hz, 2H, CH₂), 6.73-6.80 (t, 2H Ar), 7.42-7.53 (m, 2H Ar), 7.64-7.68 (m, 1H Ar), 7.72-7.75 (m, 2H Ar), 8.42 (s, 1H exchangeable, —NH).

3-(7-methoxy-2-oxo-2,3-dihydro-1H-1,4-benzodiazepin-5-yl)benzonitrile, 28c

By replacing 3-(2-amino-4-methoxybenzoyl)benzonitrile 27a in example 28a by 3-(2-amino-5-methoxybenzoyl)benzonitrile 27b and proceeding in the same manner, the above product was obtained. Yield: 35%. ¹H NMR (CDCl₃, 300 MHz): d 3.76 (s, 3H, OCH₃), 4.35 (s, 2H, CH₂), 6.70 (s, 1H Ar), 7.12-7.13 (m, 1H Ar), 7.50-7.55 (t, 1H Ar), 7.73-7.76 (m, 1H Ar), 7.84-7.87 (m, 1H Ar), 7.92 (s, 1H Ar), 8.51 (s, 1H exchangeable, —NH).

6-methoxy-5-phenyl-1,3-dihydro-2H-1,4-benzodiazepin-2-one, 28d

By replacing 3-(2-amino-4-methoxybenzoyl)benzonitrile 27a in example 28a by (2-amino-6-methoxyphenyl)(phenyl)methanone 27e and proceeding in the same manner, the above product was obtained. Yield: 68%. ¹H NMR (CDCl₃, 200 MHz): d 3.48 (s, 3H, OCH₃), 4.34 (AB system, ?d=0.85, J_{AB}=11 Hz, 2H, CH₂), 6.69-6.80 (m, 2H Ar), 7.26-7.48 (m, 6H Ar), 8.76 (s, 1H exchangeable, —NH).

7-methoxy-5-phenyl-1,3-dihydro-2H-1,4-benzodiazepin-2-one, 28e

By replacing 3-(2-amino-4-methoxybenzoyl)benzonitrile 27a in example 28a by (2-amino-4-methoxyphenyl)(phenyl)methanone 27d and proceeding in the same manner, the above product was obtained. Yield: 32%. 1H NMR (CDCl₃, 200 MHz): d 3.72 (s, 3H, OCH₃), 4.32 (s, 2H, CH₂), 6.78 (s, 1H Ar), 7.08 (m, 2H Ar), 7.33-7.48 (m, 3H Ar), 7.55-7.60 (m, 2H Ar), 8.86 (s, 1H exchangeable, —NH).

9-bromo-7,8-dimethoxy-5-phenyl-1,3-dihydro-2H-1,4-benzodiazepin-2-one, 28f

By replacing 3-(2-amino-4-methoxybenzoyl)benzonitrile 27a in example 28a by (2-amino-3-bromo-4,5-dimethoxyphenyl)(phenyl)methanone 27f, and ethyl glycinate by methyl glycinate, and proceeding in the same manner, the above product was obtained. Yield: 40%. ¹H NMR (CDCl₃, 300 MHz): d 3.74 (s, 3H, OCH₃), 3.98 (s, 3H, OCH₃), 4.32 (m, 2H, CH₂), 6.78 (s, 1H Ar), 7.41-7.67 (m, 6H, 1NH +5H Ar). Mass: (M+H)⁺=375.05+377.03.

3-(6,8-dimethoxy-2-oxo-2,3-dihydro-1H-1,4-benzodiazepin-5-yl)benzonitrfle, 28g

By replacing 3-(2-amino-4,5-dimethoxybenzoyl)benzonitrile 2a in example 3a by 3-(2-amino-4,6-dimethoxybenzoyl)benzonitrile 27g and proceeding in the same manner, the above product was obtained. Yield: 42%. ¹H NMR (DMSO, 300 MHz): d 3.46 (s, 3H, OCH₃), 3.86 (s, 3H, OCH₃), 4.18 (AB system, ?d=0.6, J_{AB}=10 Hz, 2H, NCH₂), 6.45 (s, 1H, 1H Ar), 6.49 (s, 1H Ar), 7.58-7.89 (m, 4H Ar), 10.44 (s, 1H, NH).

3-(7-bromo-6,8-dimethoxy-2-oxo-2,3-dihydro-1H-1,4-benzodiazepin-5-yl)benzonitrile, 28h

By replacing 3-(2-amino-4,5-dimethoxybenzoyl)benzonitrile 2a in example 3a by 3-(6-amino-3-bromo-2,4-dimethoxybenzoyl)benzonitrile 27h and proceeding in the same manner, the above product was obtained. Yield: 24%. ¹H NMR (DMSO, 200 MHz): d 3.32 (s, 2H, NCH₂), 3.61 (s, 3H, OCH₃), 3.98 (s, 3H, OCH₃), 6.41 (s, 1H, 1H Ar), 7.49-8.16 (m, 4H Ar), 9.80 (s, 1H, NH).

3-(8-methoxy-1-methyl-2-oxo-2,3-dihydro-1H-1,4-benzodiazepin-5-yl)benzonitrie, 29a

By replacing 3-(7,8-dimethoxy-2-oxo-2,3-dihydro-1H-1,4-benzodiazepin-5-yl)-benzonitrile 3a in example 4k by 3-(8-methoxy-2-oxo-2,3-dihydro-1H-1,4-benzodiazepin-5-yl)benzonitrile 28a and proceeding in the same manner, the above product was obtained. Yield: 57%. ¹H NMR (CDCl₃, 200 MHz): d 3.40 (s, 3H, NCH₃), 3.91 (s, 3H, OCH₃), 4.30 (AB system, ?d=1.00 J_{AB}=16 Hz, 2H, CH₂), 6.74-6.84 (m, 2H Ar), 7.13-7.18 (d, 1H Ar), 7.48-7.53 (t, 1H Ar), 7.70-7.74 (d, 1H Ar), 7.88 (m, 2H Ar).

3-(6,8-dimethoxy-1-methyl-2-oxo-2,3-dihydro-1H-1,4-benzodiazepin-5-yl)benzonitrile, 29b

By replacing 3-(7,8-dimethoxy-2-oxo-2,3-dihydro-1H-1,4-benzodiazepin-5-yl)-benzonitrile 3a in example 4a by 3-(6,8-dimethoxy-2-oxo-2,3-dihydro-1H-1,4-benzodiazepin-5-yl)benzonitrile 28g and proceeding in the same manner, the above product was obtained. Yield: 77%. ¹H NMR (CDCl₃, 300 MHz): d 3.38 (s, 3H, NCH₃), 3.53 (s, 3H, OCH₃), 3.93 (s, 3H, OCH₃), 4.35 (AB system, ?d=1.00 J_{AB}=15 Hz, 2H, CH₂), 6.34 (s, 1H Ar), 6.47 (s, 1H Ar), 7.44-7.86 (m, 1H Ar).

3-(7-bromo-6,8-dimethoxy-1-methyl-2-oxo-2,3-dihydro-1H-1,4-benzodiazepin-5-yl)benzonitrile, 29c

By replacing 3-(7,8-dimethoxy-2-oxo-2,3-dihydro-1H-1,4-benzodiazepin-5-yl)-benzonitrile 3a in example 4a by 3-(7-bromo-6,8-dimethoxy-2-oxo-2,3-dihydrp-1H-1,4-benzodiazepin-5-yl)benzonitrile 28h and proceeding in the same manner, the above product was obtained. Yield: 20%. ¹H NMR (CDCl₃, 300 MHz): d 3.29 (s, 3H, NCH₃), 3.49 (s, 3H, OCH₃), 3.94 (s, 3H, OCH₃), 4.37 (broad s, 2H, CH₂), 6.25 (s, 1H Ar), 7.44-7.75 (m, 4H Ar).

Synthetic route of substituted phenyl meta carboxamides of type 36.

3-(2-amino-4-hydroxy-5-methoxybenzoyl)benzoic acid, 32a

A mixture of 1.5 g (5.31 mmoles) of (2-amino-3,4-dimethoxybenzoyl)benzonitrile 2a, 3.13 g (55.8 mmoles) of KOH in 25 ml of ethylene glycol was heated overnight at 140° C., then 150 ml of ice water were added. 0.1 N HCl was added to obtain pH 3-4. The mixture was extracted with 4×150 ml of EtOAc and dried on MgSO₄. The EtOAc was evaporated and the product was purified by chromatography on silica gel (EtOAc). Yield: 70%. ¹H NMR (CDCl₃, 300 MHz): d 3.68 (s, 3H, OCH₃), 6.31 (s, 1H Ar), 6.82 (s, 1H Ar), 7.57-8.34 (m, 4H Ar). Mass: (M +H)⁺=288.07.

3-(7-methoxy-8-hydroxy-2-oxo-2,3-dihydro-1H-1,4-benzodiazepin-5-yl)benzoic acid, 33a

A mixture of 300 mg (1 mmole) of 3-(2-amino-4-hydroxy-5-methoxybenzoyl)benzoic acid 32a, 150 mg (2 mmoles) of ethyl glycinate.HCl, and 5 ml of anhydrous pyridine was heated under reflux under an inert atmosphere for 36 hours. Four 100 mg fractions (0.79 mmol) of ethyl glycinate.HCl were added every 6 hours. The reaction was brought to room temperature and evaporated to dryness. 100 ml of ice water were added and the solution was filtered then washed with cold water, with EtOH, then with Et₂O, and dried. Yield: 45%. The product was used in the next reaction without further purification.

3-(7,8-dimethoxy-1-methyl-2-oxo-2,3-dihydro-1H-1,4-benzodiazepin-5-yl)methyl benzoate, 34a

390 mg (9.6 mmoles) of 60% NaH in oil were added at 0° C. in an inert atmosphere to a solution of 1 g (3.2 mmoles) of 3-(7-methoxy-8-hydroxy-2-oxo-2,3-dihydro-1H-1,4-benzodiazepin-5-yl) benzoic acid 33a in 10 ml of DMF and stirred at room temperature for 1 hour. At 0° C., 600 μl of iodomethane were added dropwise and the reaction was stirred overnight at room temperature. 200 ml of water were then added and the reaction was extracted with 3×200 ml of EtOAc and dried on MgSO₄. The EtOAc was evaporated and the product was purified by chromatography on silica gel (EtOAc, then EtOAc/CH₂C1₂/EtOH, 5:4:1). Yield: 50%. ¹H NMR (CDCl₃, 300 MHz): d 3.42 (s, 3H, NCH₃), 3.74 (s, 3H, OCH₃), 3.93 (s, 3H, COOCH₃), 4.00 (s, 3H, OCH₃), 4.33 (AB system, ?d=1.01, J_{AB}=8.3, 2H, CH₂), 6.65 (s, 1H Ar), 6.81 (s, 1H Ar), 7.49-8.27 (m, 4H Ar). Mass: (M +H)⁺=369.1.

3-(7,8-dimethoxy-1-methyl-2-oxo-2,3-dihydro-1H-1,4-benzodiazeepin-5-yl)benzoic acid, 35a

300 mg of KOH pellets were added at 0° C. to a solution of 1.8 g (4.8 mmoles) of 3-(7,8-dimethoxy-1-methyl-2-oxo-2,3-dihydro-1H-1,4-benzodiazepin-5-yl)methyl benzoate 34a in 75 ml of MeOH and 25 ml of water. The solution was heated at 60° C. for 1 hour. The methanol was evaporated, 100 ml of ice water were added and the solution was then acidified to pH 2-3 by dropwise addition of IN HCl. The solution was extracted with 4×150 ml of EtOAc and dried on MgSO₄. The EtOAc was evaporated. Yield 50%. ¹H NMR (CDCl₃, 300 MHz): d 3.44 (s, 3H, NCH₃), 3.75 (s, 3H, OCH₃), 4.00 (s, 3H, OCH₃), 4.37 (AB system, ?d=1.07, J_{AB}=10.9, 2H, CH₂), 6.66 (s, 1H Ar), 6.81 (s, 1H Ar), 7.50-8.39 (m, 4H Ar). Mass: (M +H)+=369.1.

3-(7,8-dimethoxy-1-methyl-2-oxo-2,3-dihydro-1H-1,4-benzodiazepin-5-yl)N-isopropylbenzamide, 36a

To a solution of 100 mg (0.28 mmole) of 3-(7,8-dimethoxy-1-methyl-1,3-dihydro-2H-1,4-benzodiazepin-5-yl)benzoic acid 35a and 17 mg (0.29 mmole) of isopropylamine in 4 ml of DMF, 117 mg (1.17 mmoles) of N-methylmorpholine were added followed by 192 mg (0.43 mmole) of BOP. The mixture was stirred overnight at room temperature. 100 ml of water were added and the solution was extracted with 3×100 ml of CH₂Cl₂ and dried on MgSO₄. The CH₂C₁₂ was evaporated and the product was purified by chromatography on silica gel (EtOAc/hexane, 3:1 followed by EtOAc). Yield: 75%. 1H NMR (DMSO, 300 MHz): d 1.27 (d, J₁₂=6.5, 6H, CH(CH₃)₂), 3.40 (s, 3H, NCH₃), 3.72 (s, 3H, OCH₃), 3.98 (s, 3H, OCH₃),4.10 (m, J₂₁=6.5, J₂₃=7.2, 1H, CH), 4.32 (AB system, ?d=0.99, J_{AB}=10.3, 2H, CH₂), 6.20 (d, J₃₂ =7.2, 1H exchangeable, iprNH), 6.63 (s, 1H Ar), 6.78 (s, 1H Ar), 7.42-8.08 (m, 4H Ar). Mass: (M+H)⁺=396.16.

N-benzyl-3-(7,8-dimethoxy-1-methyl-2-oxo-2,3-dihydro-1H-1,4-benzodiazepin-5-yl) benzamide, 36b

By replacing isopropylamine in example 36a by benzylamine and proceeding in the same manner, the above product was obtained. Yield: 80%. ¹H NMR (CDCl₃, 300 MHz): d 3.35 (s, 3H, NCH₃), 3.69 (s, 3H, OCH₃), 3.95 (s, 3H, OCH₃), 4.23 (AB system, ?d=0.97, J_{AB}=10.3, 2H, CH₂), 4.60 (m, 2H, PhCH₂), 6.60 (s, 1H Ar), 6.76 (s, 1H Ar), 7.15 (m, 1H exchangeable, BnNH), 7.27-8.12 (m, 9H Ar). Mass: (M+H)⁺=444.20.

N-(6-amino-6-oxohexyl)-3-(7,8-dimethoxy-1-methyl-2-oxo-2,3-dihydro-1H-1,4-benzodiazepin-5-yl)benzamide, 36c

To a solution of 100 mg (0.28 mmole) of 3-(7,8-dimethoxy-1-methyl-1,3-dihydro-2H-1,4-benzodiazepin-5-yl)benzoic acid 35a and 48 mg (0.29 mmole) of 5-aminopentylcarboxamide hydrochloride in 4 ml of DMF, 30 mg of triethylamine, 117 mg (1.17 mmoles) of N-methylmorpholine were added followed by 192 mg (0.43 mmole) of BOP. The reaction was stirred overnight at room temperature. 100 ml of water were added and the reaction was extracted with 3×100 ml of CH₂Cl₂, then dried on MgSO₄. The CH₂Cl₂ was evaporated and the product was purified by chromatography on silica gel (EtOAc/CH₂Cl₂/EtOH, 5:4:1).Yield: 75%. ¹H NMR (CDCl₃, 200 MHz): d 1.66-1.73 (m, 6H, (CH₂)₃), 2.28 (m, 2H, COCH₂), 3.44-3.51 (m, 5H, NHCH₂ +NCH₃), 3.77 (s, 3H, OCH₃), 4.02 (s, 3H, OCH₃), 4.33 (AB system, ?d=0.97, J_{AB}=10.5, 2H, CH₂), 5.70 (broad s, 2H exchangeable, CONH₂), 6.69 (m, 2H, CONH +1H Ar), 6.82 (s, 1H Ar), 7.45-8.20 (m, 4H Ar). Mass: (M+H)⁺=467.2.

3-(7,8-dimethoxy-1-methyl-2-oxo-2,3-dihydro-1H-1,4-benzodiazepin-5-yl)-N,N-dimethylbenzamide, 36d

By replacing isopropylamine in example 36a by dimethylamine and proceeding in the same manner, the above product was obtained. Yield: 90%. ¹H NMR (CDCl₃, 300 MHz): d 3.01 (s, 3H, N(CH₃)₂), 3.13 (s, 3H, N(CH₃)₂), 3.43 (s, 3H, NCH₃), 3.77-3.85 (m, 4H, 1HCH₂ +OCH₃), 4.01 (s, 3H, OCH₃), 4.82 (m, 1HCH₂), 6.70 (s, 1H Ar), 6.81 (s, 1H Ar), 7.46-7.77 (m, 4H Ar). Mass: (M+H)⁺=382.20.

5-{3-[(4-benzylpyperazin-1-yl)carbonyl]phenyl}7,8-dimethoxy-1-methy1-2-oxo-2,3-dihydro-1H-1,4-benzodiazepin-2-one, 36e

By replacing isopropylamine in example 36a by N-benzylpiperazine and proceeding in the same manner, the above product was obtained. Yield: 70%. ¹H NMR (CDCl₃, 300 MHz): d 2.43-2.71 (m, 4H, 2CH₂pyp), 3.43-3.57 (m, 9H, PhCH₂+2CH₂pyp +NCH₃), 3.77-3.85 (m, 4H, 1HCH₂+OCH₃), 4.01 (s, 3H, OCH₃), 4.83 (m, 1HCH₂), 6.70 (s, 1H Ar), 6.81 (s, 1H Ar), 7.30-7.77 (m, 9H Ar). Mass: (M+H)⁺=513.20.

3-(7,8-dimethoxy-1-methyl-2-oxo-2,3-dihydro-1H-1,4-benzodiazepin-5-yl)-N-(3-phenylpropyl)benzamide, 36f

By replacing isopropylamine in example 36a by 3-phenylpropylamine and proceeding in the same manner, the above product was obtained. Yield: 95%. ¹H NMR (CDCl₃, 300 MHz): d 1.97-2.04 (m, 2H, PhCH₂), 2.72-2.80 (m, 2H, CH₂), 3.44-3.53 (m, SH, NHCH₂+NCH₃), 3.76-3.86 (m, 4H, 1HCH₂ +OCH₃), 4.01 (s, 3H, OCH₃), 4.83 (m, 1HCH₂), 6.28 (broad s, 1H exchangeable, CONH), 6.66 (s, 1H Ar), 6.82 (s, 1H Ar), 7.26-8.06 (m, 4H Ar). Mass: (M+H)⁺=472.20.

Synthesis of boronics 37 not commercially available

2-hydroxy-5-iodobenzonitrile, 37a

To a solution of 2 g (16.8 mmoles) of 2-hydroxybenzonitrile in 50 ml of acetonitrile under an inert atmosphere at -20° C., 1.65 ml of trifluoromethane sulfonic acid were added followed by incremental addition of 4.5 g (20.2 mmoles) of N-iodosuccinimide. The reaction was stirred at room temperature for 12 hours, then 200 ml of water were added and the reaction was extracted with 3×150 ml of CH₂Cl₂ and dried on MgSO₄. The CH₂Cl₂ was evaporated and the product was purified by chromatography on silica gel (EtOAc/hexane, 1:4). Yield: 85%. ¹H NMR (CDCl₃, 200 MHz): d 6.78 (m, 1H Ar), 7.70-7.79 (m, 2H Ar), 8.17 (m, 1H exchangeable, OH).

5-iodo-2-[(4-methoxybenzyl)oxylbenzonitrile, 37b

A mixture of 2 g (8.16 mmoles) of 2-hydroxy-5-iodobenzonitrile 37a, 4.5 g (32.64 mmoles) of K₂CO₃, 295 mg (0.8 mmole) of tetra n-butyl ammonium iodide, 1.4 g (8.98 mmoles) of 4-methoxybenzylchloride in 75 ml of anhydrous acetone was stirred at room temperature under an inert atmosphere for 12 hours. The acetone was evaporated, 150 ml of water were added and the solution was extracted with 3×150 ml of CH₂C₁₂ and dried on MgSO₄. The CH₂Cl₂ was evaporated, the residue was triturated in 20 ml of EtOAc, filtered, rinsed with a minimum of EtOAc, and dried. Yield: 75%. ¹H NMR (CDCl₃, 300 MHz): d 3.81 (s, 3H, OCH₃), 5.13 (s, 2H, CH₂Ph), 6.79 (m, 1H Ar), 6.92 (m, 2HBn), 7.35 (m, 2HBn), 7.72-7.82 (m, 2H Ar).

3-cyano-4-[(4-methoxybenzyl)oxylphenylboronic acid, 37c

1 ml of 1.7 M terBuLi in pentane cooled to -78° C. was added by cannulation at −78° C. under an inert atmosphere to a solution of 300 mg (0.82 mmole) of 5-iodo-2-[(4-methoxybenzyl)oxy]benzonitrile 37b in 10 ml of anhydrous THF. The reaction was stirred for 30 minutes, then a solution of 930 μl of trimethylborate in 10 ml of anhydrous THF cooled to −78° C. was added by cannulation. The solution was allowed to return to room temperature overnight. 50 ml of ice water were added and the solution was extracted with 4×50 ml of EtOAc and dried on MgSO₄. The EtOAc was evaporated and the product purified by flash chromatography on silica (EtOAc, then EtOAc/CH₂Cl₂/EtOH 5:4:1). Yield: 65%.

### Example 2

### Pharmacological Activity: Inhibition of Phosphodiesterases

2.1. Isolation of phosphodiesterases from smooth muscle

A 3 g segment of bovine aortic media cut into pieces with scissors was homogenized with an ultra-turrax then a potter glass/glass homogenizer in 7 volumes by weight of buffer A containing a protease inhibitor cocktail (20 mM Tris-HCl, 0.25 M saccharose, 2 mM magnesium acetate, 1 mM dithiothreitol, 5 mM EGTA, 2000 U/ml aprotinin, 10 mg/l leupeptin and 10 mg/l soya trypsic inhibitor). The homogenizate was centrifuged at 105,000 g for 1 hour. The supernatant was loaded on a DEAE-Sephacel column (15 x 1.6 cm) pre-equilibrated with buffer B (buffer A without the saccharose, EGTA and protease inhibitors). The column was washed until there was no detectable absorption at 280 nm, then eluted with a linear gradient of NaCl (0-0.5 M) in buffer B. 3-ml fractions were collected and enzyme activity was determined under the conditions described hereinbelow to localize the different enzymes PDE1, PDE3, PDE4 and PDE5 which were aliquoted and frozen at −80° C. (Lugnier et al., Biochem. Phamacol., 1986, 35: 1746-1751). PDE2 was prepared from bovine endothelial cells by the same methods (Lugnier and Schini, Biochem. Pharmacol., 1990, 39: 75-84).

2.2. Protocol for measurinp, hosphodiesterase activity

Cyclic nucleotide phosphodiesterase activity was determined by a radioenzymatic method using tritium-labelled cyclic GMP or AMP (1 μM) as substrate (Lugnier et al., 1986). ³H-labelled adenosine or guanosine monophosphate formed by hydrolysis of the radiolabelled cyclic nucleotide was then converted to ³H-labelled adenosine or guanosine in a second reaction with one nucleotidase in excess. The nucleoside formed was separated from the nucleotides by anion exchange chromatography. Nucleoside radioactivity was determined by liquid scintillation counting. Enzymatic incubations were carried out under conditions allowing no more than 15% hydrolysis of the substrate; each point was performed in duplicate.

2.2.1. Determination of inhibition of PDE2.

The concentration of substance which inhibits enzymatic activity by 50% (IC₅₀) at 1 μM cyclic AMP was calculated by nonlinear regression from the experimental values of hydrolysis rate (Prism, GraphPad).

2.2.2. Selectivity

The activity of the compounds was evaluated on other phosphodiesterase isoforms, particularly basal state or calmodulin-activated PDEl from vascular smooth muscle, PDE3, PDE4 and PDE5 from vascular smooth muscle.

The results obtained are presented in Tables 1 and 2 hereinbelow and are expressed as the percentage inhibition of enzymatic activity produced by 10 μmol of the test compound.

TABLE 1
Compound represented by formula (I)

| | PDE2 IC₅₀ (μM) or | | PDE2 IC₅₀ (μM) or |
|---|---|---|---|
| | percentage | | percentage |
| Compound | inhibition at 10 μM | Compound | inhibition at 10 μM |
| 3a | 22 | 6j | 0.71 |
| 3d | 22% | 6k | 69.7% |
| 4a | 6.7 | 6l | 77.5% |
| 4c | 35.3% | 6m | 82.3% |
| 4d | 47.6% | 6n | 84.6% |
| 4e | 13.9% | 6o | 79.3% |
| 4f | 17.1% | | |
| 4g | 14.3% | 7b | 5.5 |
| 4h | 16% | 7c | 41% |
| 4i | 15.7% | 7d | 33.8% |
| 4j | 5.6% | 8a | 9 |
| 4k | 75.9% | 8b | 27.2% |
| 4l | 72.1% | 9a | 85% |
| 4m | 1.5 | 9b | 91.4% |
| 4n | 3.7 | | |
| 4p | 1.8 | | |
| 4q | 32% | | |
| 4r | 34% | 10d | 5.5% |
| 4s | 14% | 11a | 43% |
| 5a | 1.5 | 11b | 69.3% |
| 5b | 2.1 | 12a | 16% |
| 5c | 53.3% | | |
| 5d | 19.2% | 17b | 1.5 |
| 5e | 6.6 | 17c | 6.1 |
| 5f | 12.6 | 17d | |
| 5g | 24.6 | 17e | 6.7 |
| 5h | 0% | 17f | 4.7 |
| 5i | 0% | | |
| 5j | 4.5 | 17h | 9.2% |
| 5k | 67.8% | 17i | 40% |
| 5l | 14 | 17j | 7.0 |
| 5m | 5.9% | 17k | 3.7 |
| 5n | | 17l | 5.0 |
| 5o | | 17m | 4.8 |
| 6a | 8.4 | 17n | 38% |
| 6b | 1.06 | 17o | 7.8 |
| 6c | 4.3 | 22b | 3.8% |
| 6d | 2.4 | 23b | 20.1% |
| 6e | 0.36 | 23d | |
| 6h | | 24b | 3.3 |
| 6i | 5.6 | 25a | 0% |
| 25b | 22.9% | 28h | |
| 25c | 3.4 | 29a | 35 |
| 25d | 2.4% | 29b | |
| 25e | 5.0 | 29c | |
| 25f | 0% | 34a | 2.6 |
| 25g | 14.5% | 35a | 26% |
| 28a | 52.5% = 65 | 36a | 75.9% |
| 28b | 34.5% = 88 | 36b | 3.1 |
| 28c | 46.5% | 36c | 46% |
| 28d | 4.7% | 36d | 17% |
| 28e | 8.8% | 36e | 27% |
| 28f | 13.5% | 36f | 2.8 |
| 28g | | | |

TABLE 2
Selectivity

| | IC₅₀ (μM) or percentage | | | | |
|---|---|---|---|---|---|
| | inhibition at 10 μM | | | | |
| Compound | PDE1 | PDE2 | PDE3 | PDE4 | PDE5 |
| 4m | | 1.5 | | | |
| 4p | 25% | 1.8 | 58% | 19% | 26% |
| 5a | 13.2% | 1.5 | 5% | 16.2% | 17.6% |
| 5b | | | 2.1 | | 21.6% |
| 6b | | 1.06 | | | |
| 6d | | 2.4 | | 55.7% | |
| 6e | | 0.36 | | | |
| 6j | | 0.71 | 5 | 2.8 | |
| 6m | | 82.3% | | 37.3% | |
| 6n | | 84.6% | | 58.7% | |
| 7a | | 3.13 | | 6.52 | |
| 9b | | 91.4% | | | |
| 17b | 10% | 1.5 | 36% | 8% | 14% |

All the compounds tested showed potent inhibition of PDE2. The preferred compounds according to the invention have an excellent potency and selectivity profile for phosphodiesterase 2, in so far as said compounds are weaker inhibitors of the other PDEs, particularly PDE3.

### Example 3

### Bhavioral Tests

Compound 5a was evaluated in different behavioral tests

3.1 Elevated plus maze test

This test was validated in the rat by Pellow in 1986 and in the mouse by Lister in 1987. It is based on an aversion to open spaces: the open arms elicit anxiety in the animals while the closed arms represent safety. By recording the frequency of entry into each arm, this test evaluates the anxiolytic effect of a molecule in comparison with a reference compound such as buspirone.

Ten to eleven-week old Balb/c or Swiss mice were used for the test. Mice were randomly divided into a control group (treated with the vehicle) and other groups treated with the test compounds.

The test apparatus was a PVC maze with a plexiglass lid, divided into four equivalent exploration arms (45×10 cm), all interconnected by a small platform (10×10 cm). The apparatus was placed 66 crn above the floor. Two arms were opened and the other two closed with a wall (height: 30 cm).

After administration, the mouse was placed on the platform opposite the closed arm. The number of entries and the time spent in each open arm were recorded over 8 minutes.

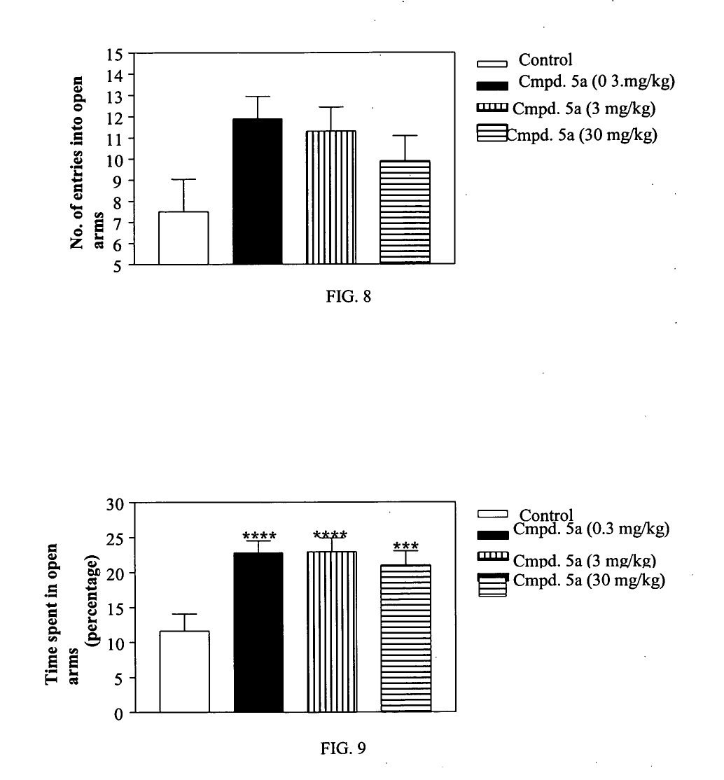

The treatment was administered 1 hour before the test. The compounds were given orally at different doses. The results are shown in FIGS. 8 and 9. N=10; *** p<0.005 and **** p<0.001 (versus control; Dunnett test).

A significant difference between the groups was observed, in particular with regard to the percentage of time spent in the open arms. Mice treated With compound 5a spent more time in the open arms, and this at all doses tested.

3.2 Swim test

This test is based on the induction of alternative behavior in rodents subjected to an acute stress. In this model, the rat or mouse placed in a water-filled cylinder adopts a particular state of immobility. Onset of said immobility is delayed by antidepressants administered acutely or in repeated doses.

Wistar rats or Swiss mice were used. Animals were isolated for one week with a reverse light/dark cycle, then placed in the water-filled cylinder for 6 minutes. The total immobility time was recorded during the last 4 minutes. The treatment was administered 20 minutes before the test. Four groups were used to test three different doses: one control group treated with the vehicle, and one group for the three doses.

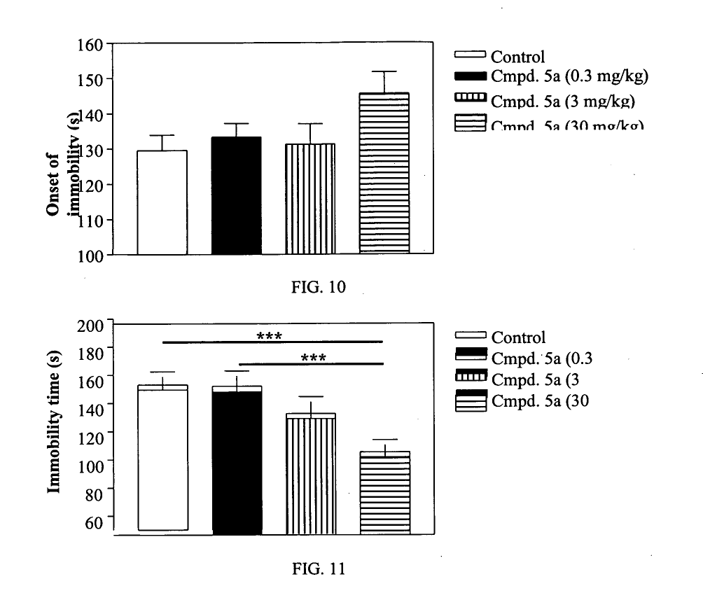

The results are shown in FIGS. 10 and 11. Two parameters were recorded: onset of shorter immobility time. N=10; *** p<0.005 (Dunnett test).

The statistical analyses showed a significant difference between the groups for total immobility time (p=0.007). Mice treated with compound 5a at 3 and 30 mg/kg had a shorter immobility time than the control group and the group treated with compound 5a at 0.3 mg/kg.

3.3 Light/dark test

This light/dark test is based on the natural tendency of rodents to prefer a dark environment and thereby evaluates the emotional responses of the animals in a situation of bright light. This procedure is suitable for assessing the state of anxiety elicited by anxiety stimuli (Lister's group, 1990). Mice placed in the apparatus, while showing a preference for the dark zone, nonetheless explore the light zone. This procedure was validated in 1990 by Misslin et al., who demonstrated that the anxiolytic and angiogenic properties of different substances act on the serotoninergic system or on benzodiazepine GABA receptor complexes.

Ten to eleven-week old Balb/c or Swiss mice were used. Mice were randomly divided into a control group (treated with the vehicle) and other groups treated with the test compounds.

The test apparatus consisted of two PVC compartments (20×20×14 cm). with a plexiglass lid. One of the compartments was dark. A 100W light bulb was placed 15 cm above the other compartment, emitting the only light in the room (approximately 4400 lux). An opaque plastic tunnel separated the light and dark compartments.

The animal was placed in the light compartment with its head pointed towards the tunnel. The time spent in the light compartment and the number of entries into the light compartment were recorded for 5 minutes after the first entry into the dark zone. The test compound or the control treatment were administered orally 1 hour before the test.

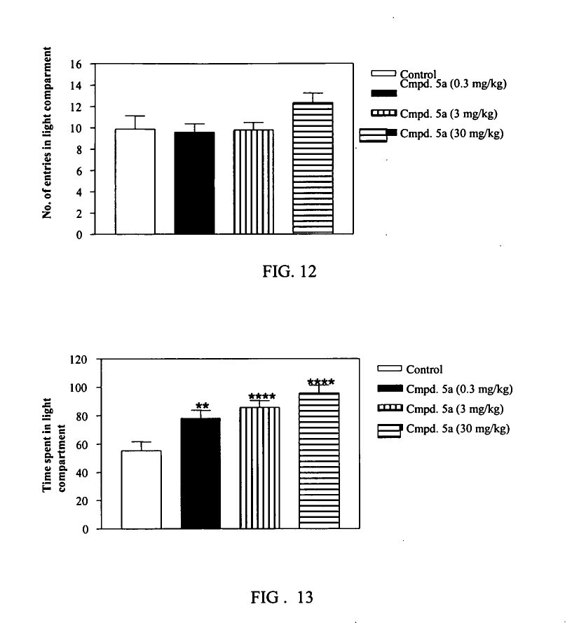

The results are shown in FIGS. 12 and 13. N=10; ** p<0.01; **** p<0.001 (Dunnett test versus control).

A significant difference was observed between the groups for the time spent in the light compartment (p<0.001). Mice treated with compound 5a at 0.3, 3 and 30 mg/kg spent significantly more time in the light compartment as compared with controls (p<0.01, control versus Dunnett test).

Together these results confirm the anxiolytic and antidepressant effect of the inventive compounds and in particular of compound Sa, in particular at the doses tested.

1-28. (canceled)

29. A method for treating pathologies related to the nervous system (central and peripheral), in particular central, by administering to a subject in need of such treatment at least one phosphodiesterase 2 inhibitor.

30. The method according to claim 29, wherein the pathologies are due to a deregulation of the function of a neurotransmitter or to a deficiency in the release of a neurotransmitter, in particular of dopamine.

31. The method according to claim 29, wherein the pathologies are selected in the group consisting of depression, schizophrenia, anxiety, bipolar disorder, attention deficit disorders, sleep disorders, obsessive compulsive disorder, fibromyalgia, Tourette's syndrome, pharmacodependence, epilepsy, Alzheimer's disease, Parkinson's disease, amyotrophic lateral sclerosis, multiple sclerosis, obesity and Lewy body dementia.

32. The method according to claim 29, wherein the pathologies involve the peripheral nervous system and the peripheral organs, in particular pathologies of the type reduced natriuria, acute renal failure, hepatic dysfunction, acute hepatic failure, in particular due to age.

33. The method according to claim 29, wherein the pathologies are due to or involve dysfunctions of prolactin secretion, in particular selected in the group consisting of restless legs syndrome, rheumatismal, allergic or auto-inflammatory disorders, more particularly selected in the group consisting of rheumatoid arthritis, rhinitis or asthma.

34. The method according to claim 29, wherein the pathologies are selected in the group consisting of anxiety, depression and schizophrenia.

35. The method according to claim 29, wherein the PDE2 inhibitors are selected in the group consisting of 1,4-benzodiazepine derivatives.

36. A compound represented by general formula (I) 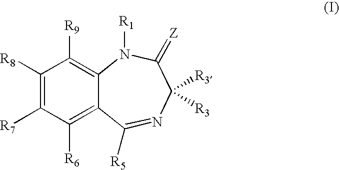
in which:
Z represents an oxygen or sulfur atom or a NR₂ group,
R₁ is the hydrogen atom, a (C₁-C₆) alkyl group, a (C₆-C₁₈)aryl group or a (C₁-C₆)alkyl(C₆-C₁₈)aryl or (C₆-C₁₈)aryl(C₁-C₄)alkyl group,
R₂ is a hydrogen atom, a (C₁-C₆) alkyl group, a (C₆-C₁₈)aryl group or a (C₁-C₆)alkyl(C₆-C₁₈)aryl or (C₆-C₁₈)aryl(C₁-C₄)alkyl group,
R₁ and R₂ taken together can optionally form a linear or branched hydrocarbon chain having from 2 to 6 carbon atoms, possibly containing one or several other double bonds and/or possibly interrupted by an oxygen, sulfur or nitrogen atom,
R₃ and R_{3′}, which are the same or different, represent the hydrogen atom, a (C₁-C₁₂) alkyl, (C₃-C₆) cycloalkyl, (C₆-C₁₈) aryl, (C₆-C₁₈)aryl(C₁-C₄)alkyl, (C₁-C₁₂)alkyl(C₆-C₁₈)aryl group or a (C₅-C₁₈) heterocycle, aromatic or not, containing 1 to 3 heteroatoms, a NO₂, CF₃, CN, NR′R″, SR′, OR′, COOR′, CONR′R″ or NHCOR′R″ group, R′ and R″, independently of each another, being selected in the group consisting of the hydrogen atom, a (C₁-C₆) alkyl, (C₃-C₆) cycloalkyl, (C₆-C₁₂) aryl group and a (C₅-C₁₂) heterocycle, aromatic or not, containing 1 to 3 heteroatoms;
R₅ represents a phenyl group substituted at least in position 3, a naphthyl group, a (C₅-C₁₈) heterocycle, aromatic or not, containing 1 to 3 heteroatoms, selected in the group consisting of the pyridyl, isoquinolyl, quinolyl and piperazinyl group, provided that when R₅ is a naphthyl group substituted in position 6, then the latter is not attached to the rest of the molecule in position 2, or when R₅ is a pyridyl group, then it is not attached to the rest of the molecule in position 4, or when R₅ is a tetrahydro 1,2,3,4-isoquinolyl group, then it is not attached to the rest of the molecule in position 2,
when R₅ represents a phenyl group substituted at least in position 3, said substituent being selected in the group consisting of: an alkyl, halogenoalkyl, cycloalkyl, alkenyl, alkynyl, aralkyl, aryl, heterocycle, heterocycloalkyl group, a OH, ═O, NO₂, NH₂, CN, CF₃, COR′, COOR′, (C₁-C₆)alkoxy, (di)(C₁-C₆)alkylamino, NHCOR′, CONR′R″ group, in which R′ and R″ are such as defined hereinabove, CHO, CONH2, phenyl optionally substituted, in particular by an acetyl group, by a halogen atom (Cl), by a CONH2 group or by a CN, prop-1-ynyl optionally substituted, in particular by a benzyloxy or tert-butyl carbamate, hex-1-ynyl optionally substituted, in particular by a CN or NH2, pentyl optionally substituted, in particular by a CONH2, hexyl, piperidinyl group optionally substituted, in particuar by a prop-1-ynyl, benzylaminomethyl, acetamide (CH3CONH), aminomethyl, NH2CS—, 4-phenyl-1, 3-thiazol-2-yl, —CONHBenzyl, —COOEthyl, —CONHiPropyl, —CONH—(CH2)n—CONH2 (n representing a whole number from 1 to 6), —CONR′R″ group, with R′ and R″, which are the same or different, representing a C₁-C₆ alkyl group or a hydrogen atom, -(4-benzylpyperazin-1-yl)carbonyl, —CONH—(CH2)n-phenyl (n representing a whole number from 1 to 6), imidazolyl, piperazinyl optionally substituted, in particular by a phenyl group,
R₇ and R₈, independently of each other, are selected in the group consisting of the hydrogen atom, a halogen atom or a OR₁₀, group in which R₁₀ represents a hydrogen atom, a (C₁-C₆) alkyl, (C₃-C₆) cycloalkyl, (C₆-Cl₂) aryl group, or a (C₅-C₁₂) heterocycle, aromatic or not, containing 1 to 3 heteroatoms, at least one of the groups R₇ and R₈ representing a OR₁₀ group such as defined hereinabove,
R₆ and R₉, independently of each other, are selected in the group consisting of the hydrogen atom, a halogen atom, an alkyl, cycloalkyl, alkenyl, alkynyl group, an aryl, aralkyl, heterocycle, heterocycloalkyl group and a OR₁₀ group, R₁₀ being such as defined hereinabove,
the alkyl, cycloalkyl, alkenyl, alkynyl, aralkyl, aryl, phenyl, naphthyl, heterocycle, heterocycloalkyl group or the hydrocarbon chain defined hereinabove being optionally substituted by one or more substituents, which are the same or different, preferably selected in the group consisting of a halogen atom, an alkyl, halogenoalkyl, cycloalkyl, alkenyl, alkynyl, aralkyl, aryl, heterocycle, heterocycloalkyl group, a OH, ═O, NO₂, NH₂, CN, CF₃, COR′, COOR′, (C₁-C₆)alkoxy, (di)(C₁-C₆)alkylamino, NHCOR′ and CONR′R″ group, in which R′ and R″ are such as defined hereinabove, the substitutents also being optionally substituted,
and one of the salts thereof,
with the exception of compounds represented by formula (I) in which
R1 l is an alkyl group, R3 and R′3 are hydrogen atoms, R6 and R9 are hydrogen atoms, R5 is a phenyl group substituted at least in position 3 by a methoxy group,
R1 is an alkyl group or a hydrogen atom, R3 and R′3 are hydrogen atoms, R6 and R9 are hydrogen atoms, R5 is a phenyl group substituted only in position 3 by a chlorine or bromine atom,
R1 is an alkyl group, R3 and R′3 are hydrogen atoms, R6 and R9 are hydrogen atoms, R5 is a phenyl group substituted at least in position 3 by a CH2OH group,
R1 is a hydrogen atom, R3 and R′3 are hydrogen atoms, R6 and R9 are hydrogen atoms, R5 is a phenyl group substituted only in position 3 by a CF3 group,
R1 is an alkyl group, R3 and R′3 are hydrogen atoms, R6 and R9 are hydrogen atoms, R5 is a phenyl group substituted in positions 3 and 5 by a CF3 group,
R1 is an alkyl group, R3 and R′3 are hydrogen atoms, R6 and R9 are hydrogen atoms, R7 and R8 are methoxy groups, R5 is a phenyl group substituted in positions 3 by a phenyl group,
R1 is an alkyl group, R3 and R′3 are hydrogen atoms, R6 and R9 are hydrogen atoms, R7 and R8 are methoxy groups, R5 is a phenyl group substituted in positions 3 by a phenylethynyl group.

37. The compound represented by formula (I) according to claim 36, in which R₅ is a phenyl group substituted at least in position 3.

38. The compound represented by formula (I) according to claim 36, wherein R₅ is a phenyl group substituted at least in position 3 and wherein the substituent is selected in the group consisting of: CHO, CN, CONH2, NO2, CF3, NH2, halogen atom (Cl), (C1-C6) alkyl, phenyl optionally substituted, in particular by an acetyl group, by a halogen atom (Cl), by a CONH2 group or by a CN, prop-1-ynyl group optionally substituted, in particular by a benzyloxy or tert-butyl carbamate, hex-1-ynyl group optionally substituted, in particular by a CN or NH2 group, pentyl optionally substituted, in particular by a CONH2, hexyl, piperidinyl optionally substituted, in particular by a prop-1-ynyl, benzylaminomethyl, acetamide (CH3CONH), aminomethyl, NH2CS—, 4-phenyl-1,3-thiazol-2-yl, —CONHBenzyl, —COOEthyl, —CONHiPropyl, —CONH—(CH2)n—CONH2 (n representing a whole number from 1 to 6), —CONR′R″ group, with R′ and R″, which are the same or different, representing a C1 to C6 alkyl group or a hydrogen atom, -(4-benzylpyperazin-1-yl)carbonyl, —CONH—(CH2)n-phenyl (n representing a whole number from 1 to 6), imidazolyl, piperazinyl group optionally substituted, in particular by a phenyl group.

39. The compound represented by formula (I) according to claim 36, in which R5 is a phenyl group substituted in positions 3 and 4, in particular by a hydrocarbon chain possibly containing at least one heteroatom, such as oxygen, like the methylenedioxy (—O—CH2—O—) chain forming a ring with the phenyl group to which it is attached.

40. The compound represented by formula (I) according to claim 36, in which R5 is the 3-pyridyl, 4-isoquinolyl, piperazinyl group optionally substituted, in particular in position 4 by an aryl group, such as phenyl.

41. The compound represented by formula (I) according to claim 36, in which Z represents a sulfur atom or —NR2, with in particular R2 forming a ring of the imidazole type with R1.

42. The compound represented by formula (I) according to claim 36, in which
Z is the oxygen atom, and/or
R₇ and R₈, independently of each other, represent a OR₁₀ group in which R₁₀ is a (C₁-C₆) alkyl group, preferably an ethyl or methyl group, advantageously methyl, and/or
R₇ and R₈ each represent an ethoxy or methoxy group, advantageously methoxy, or one represents a hydrogen atom and the other an ethoxy or methoxy group, advantageously methoxy, and/or
R₆ and R₉, which are the same or different, represent the hydrogen atom, a halogen atom, a phenyl group, a (C₁-C₆) alkyl group or a OR₁₀ group in which R₁₀ is a (C₁-C₆) alkyl group, preferably an ethyl or methyl group, and/or
R₃ and R₃′, which are the same or different, represent a hydrogen atom, and/or
R₁ is a (C1-C6) alkyl, (C₆-C₁₈) aryl group, such as phenyl, (C₆-C₁₈)aryl(C₁-C₄)alkyl group such as benzyl optionally substituted, or (C₁-C₁₂)alkyl(C₆-C₁₈)aryl group.

43. The compound represented by formula (I) according to claim 36, in which R₁ represents a hydrogen atom or a (C₁-C₃) alkyl, (C₆-C₁₈) aryl (for example: phenyl), (C₆-C₁₈)aryl(C₁-C₄)alkyl (for example: benzyl), (C₁-C₁₂)alkyl(C₆-C₁₈)aryl group, said group being optionally substituted.

44. The compound represented by formula (I) according to claim 36, in which R₅ is a phenyl group substituted by:
(a) one or more OR′ groups, in particular methoxy or ethoxy, or
(b) a COR′ group, in particular acetyl or aldehyde, or
(c) a CONR′R″ group, in particular CONH2, or
(d) a CN group, or
(e) a trifluoromethyl group, or
(f) an alkyl group, for example methyl, or alkynyl group, for example hexynyl or propynyl, or
(g) an aryl or heterocycle group, in particular phenyl, furyl, pyridyl, piperidine, thiazole or thienyl, said aryl or heterocycle itself being optionally substituted by one or more groups preferably selected from groups (a)-(g).

45. The compound represented by formula (I) according to claim 36, in which at least one group R₇ or R₈ represents OR₁₀ where R₁₀ represents a (C₁-C₆) alkyl or (C₃-C₆) cycloalkyl group.

46. The compound represented by formula (I) according to claim 36, in which at least one group R₇ or R₈ represents OR₁₀ where R₁₀ represents a (C₁-C₆) alkyl or (C₃-C₆) cycloalkyl group and at least one of the groups R7 and R8, advantageously both, represents a methoxy group.

47. The compound according to claim 36, wherein said compound is selected from the following compounds:
3-(7,8-dimethoxy-2-oxo-2,3-dihydro-1H-1,4-benzodiazepin-5-yl)benzonitrile, 3a
7,8-dimethoxy-[5-(3-trifluoromethyl)phenyl]-1,3-dihydro-2H-1,4-benzodiazepin-2-one, 3d
3-(7,8-dimethoxy-1-methyl-2-oxo-2,3-dihydro-1H-1,4-benzodiazepin-5-yl)-benzonitrile, 4a
3-[1-(4-chlorobenzyl)-7,8-dimethoxy-2-oxo-2,3-dihydro-1H-1,4-benzodiazepin-5-yl]-benzonitrile, 4c
3-[1-(3,4-chlorobenzyl)-7,8-dimethoxy-2-oxo-2,3-dihydro-1H-1,4-benzodiazepin-5-yl]-benzonitrile, 4d
3-[7,8-dimethoxy-1-(4-methoxybenzyl)-2-oxo-2,3-dihydro-1H-1,4-benzodiazepin-5-yl]-benzonitrile, 4e
3-[1-(3-chlorobenzyl)-7,8-dimethoxy-2-oxo-2,3-dihydro-1H-1,4-benzodiazepin-5-yl]-benzonitrile, 4f
3-{7,8-dimethoxy-2-oxo-1-[3-(trifluoromethyl)benzyl]-2,3-dihydro-1H-1,4-benzodiazepin-5-yI]-benzonitrile, 4g
3-[1-(2-chlorobenzyl)-7,8-dimethoxy-2-oxo-2,3-dihydro-1H-1,4-benzodiazepin-5-yl]-benzonitrile, 4h
3-{7,8-dimethoxy-2-oxo-1-[4-(trifluoromethyl)benzyl]-2,3-dihydro-1H-1,4-benzodiazepin-5-yl]-benzonitrile, 4i
3-[7,8-dimethoxy-2-oxo-1-(2-phenylethyl)-2,3-dihydro-1H-1,4-benzodiazepin-5-yl]-benzonitrile, 4j
3-(i -ethyl-7,8-dimethoxy-2-oxo-2,3-dihydro-1H-1,4-benzodiazepin-5-yl)benzonitrile, 4k
3-(7,8-dimethoxy-1-propyl-2-oxo-2,3-dihydro-1H-1,4-benzodiazepin-5-yl)benzonitrile, 4l
3-(i -benzyl-7,8-dimethoxy-2-oxo-2,3-dihydro-1H-1,4-benzodiazepin-5-yl)benzonitrile, 4m
ethyl[5-(3-cyanophenyl)-7,8-dimethoxy-2-oxo-2,3-dihydro-i H-1,4-benzodiazepin-1-yl]acetate, 4n
7,8-dimethoxy-i -methyl-[5-(3-trifluoromethyl)phenyl]-1,3-dihydro-2H-1,4-benzodiazepin-2-one, 4p
7,8-dimethoxy-1-ethyl-5-[3-(trifluoromethyl)phenyl]-1,3-dihydro-2H-1,4-benzodiazepin-2-one, 4q
5-[3-(trifluoromethyl)phenyl]-7,8-dimethoxy-1-n-propyl-1,3-dihydro-1,4-benzodiazepin-2-one, 4r
1-benzyl-5-[3-(trifluoromethyl)phenyl]-7,8-dimethoxy-1,3-dihydro-1,4-benzodiazepin-2-one, 4s
3-(7,8-dimethoxy-1-methyl-2-oxo-2,3-dihydro-1H-1,4-benzodiazepin-5-yl)-benzamide 5a
3-(6-bromo-7,8-dimethoxy-1-methyl-2-oxo-2,3-dihydro-1H-1,4-benzodiazepin-5-yl)benzamide, 5b
3-(7,8-dimethoxy-1-methyl-2-oxo-6-phenyl-2,3-dihydro-1H-1,4-benzodiazepin-5-yl)benzamide, 5c
3-(9-bromo-7,8-dimethoxy-1-methyl-2-oxo-2,3-dihydro-1H-1,4-benzodiazepin-5-yl)benzamide, 5d
3-(7,8-dimethoxy-2-oxo-2,3-dihydro-1H-1,4-benzodiazepin-5-yl)benzamide, 5e
3-(7,8-dimethoxy-1-propyl-2-oxo-2,3-dihydro-1H-1,4-benzodiazepin-5-yl)benzamide, 5f
3-(1-ethyl-7,8-dimethoxy-2-oxo-2,3-dihydro-1H-1,4-benzodiazepin-5-yI)benzamide, 5g
3-(1-benzyl-7,8-dimethoxy-2-oxo-2,3-dihydro-1H-1,4-benzodiazepin-5-yl)benzamide, 5h
ethyl{5-[3-(aminocarbonyl)phenyl]-7,8-dimethoxy-2-oxo-2,3-dihydro-1H-1,4-benzodiazepin-1-yl}acetate, 5i
3-(7,8-dimethoxy-1,3-dimethyl-2-oxo-2,3-dihydro-1H-1,4-benzodiazepin-5-yl) benzamide, 5j
3-[3-(3,4-dichlorobenzyl)-7,8-dimethoxy-1-methyl-2-oxo-2,3-dihydro-1H-1,4-benzodiazepin-5-yl]benzamide, 5k
3-(8-methoxy-1-methyl-2-oxo-2,3-dihydro-1H-1,4-benzodiazepin-5-yl)benzamide, 5l
3-(7,8-dimethoxy-1-methyl-2-oxo-9-phenyl-2,3-dihydro-1H-1,4-benzodiazepin-5-yl)benzamide, 5m
3-(6,8-dimethoxy-2-oxo-2,3-dihydro-1H-1,4-benzodiazepin-5-yl)benzamide, 5n
3-(6,8-dimethoxy-1-methyl-2-oxo-2,3-dihydro-1H-1,4-benzodiazepin-5-yl)benzamide, 5o
tert-butyl-3-[3-(7,8-dimethoxy-1-methyl-2-oxo-2,3-dihydro-1H-1,4-benzodiazepin-5-yl)phenyl]propynylcarbamate, 6a
7,8-dimethoxy-5-(3′-hex-1-ynylphenyl)-l -N-methyl-1,3-dihydro-2H-1,4-benzodiazepin-2-one, 6b
7,8-dimethoxy-1-methyl-5-[3-(3-piperidin-1-ylprop-1-ynyl)phenyl]-1,3-dihydro-2H-1,4-benzodiazepin-2-one, 6c
6-[3-(7,8-dimethoxy-1-methyl-2-oxo-2,3-dihydro-1H-1,4-benzodiazepin-5-yl)phenyl]hex-5-ynenitrile, 6d
7,8-dimethoxy-5-(3′-hexylphenyl)-1-N-methyl-1,3-dihydro-2H-1,4-benzodiazepin-2-one, 6e
5-[3-(3-aminopropyl)phenyl-7,8-dimethoxy-1-methyl-1,3-dihydro-2H-1,4-benzodiazepin-2-one trifluoro6acetate, 6h
6-[3-(7,8-dimethoxy-1-methyl-2-oxo-2,3-dihydro-1H-1,4-benzodiazepin-5-yl)phenyl]hexanamide, 6i
5-(4′-chloro-1,1′-biphenyl-3-yl)-7,8-dimethoxy-i -methyl-1,3-dihydro-2H-1,4-benzodiazepin-2-one, 6j
5-{3-[3-(benzyloxy)prop-1-ynyl]phenyl}-1-ethyl-7,8-dimethoxy-1,3-dihydro-2H-1,4-benzodiazepin-2-one, 6k
3′-(7,8-dimethoxy-1-methyl-2-oxo-2,3-dihydro-1H-1,4-benzodiazepin-5-yl)-1,1′-biphenyl-3-carbonitrile, 6l
3′-(7,8-dimethoxy-1-methyl-2-oxo-2,3-dihydro-1H-1,4-benzodiazepin-5-yl)-1,1′-biphenyl-4-carbonitrile, 6m
3′-(7,8-dimethoxy-1-methyl-2-oxo-2,3-dihydro-1H-1,4-benzodiazepin-5-yl)-1,1′-biphenyl-4-carboxamide, 6n
3′-(7,8-dimethoxy-1-methyl-2-oxo-2,3-dihydro-1H-1,4-benzodiazepin-5-yl)-1,1′-biphenyl-3-carboxamide, 6o
3-[3-(3,4-dichlorobenzyl)-7,8-dimethoxy-1-methyl-2-oxo-2,3-dihydro-1H-1,4-benzodiazepin-5-yl]benzonitrile, 7b
7,8-dimethoxy-1,3-dimethyl-5-(3-trifluoromethylphenyl)-1,3-dihydro-2H-1,4-benzodiazepin-2-one, 7c
3-(7,8-dimethoxy-1,3-dimethyl-2-oxo-2,3-dihydro-1H-1,4-benzodiazepin-5-yl)benzonitrile, 7d
5-[3-(aminomethyl)phenyl]-7,8-dimethoxy-1-methyl-1,3-dihydro-2H-1,4-benzodiazepin-2-one, 8a
N-[3-(7,8-dimethoxy-1-methyl-2-oxo-2,3-dihydro-1H-1,4-benzodiazepin-5-yl)benzyl]acetamide, 8b
3-(7,8-dimethoxy-1-methyl-2-oxo-2,3-dihydro-1H-1,4-benzodiazepin-5-yl)thiobenzamide, 9a
7,8-dimethoxy-1-methyl-5-[3-(4-phenyl-1,3-thiazol-2-yl)phenyl]-1,3-dihydro-2H-1,4-benzodiazepin-2-one, 9b
5-(3-cyanophenyl)-7,8-dimethoxy-1,3-dihydro-2H-1,4-benzodiazepin-2-thione, 10d
3-(8,9-dimethoxy-4H-imidazo[1,2-a][1,4]benzodiazepin-6-yl)benzonitrile, 11 a
3-(8,9-dimethoxy-4H-imidazo[1,2-a][1,4]benzodiazepin-6-yl)benzamide, 11b
3-(7,8-dimethoxy-2-methylamino-1,3-dihydro-3H-1,4-benzodiazepin-5-yl)benzonitrile, 12a
7,8-dimethoxy-1-methyl-5-(3-pyridyl)-1,3-dihydro-1,4-benzodiazepin-2-one, 17b
7,8-dimethoxy-1-methyl-5-(3-nitrophenyl)-1,3-dihydro-1,4-benzodiazepin-2-one, 17c
5-(7,8-dimethoxy-1-methyl-2-oxo-2,3-dihydro-1H-1,4-benzodiazepin-5-yl)-2-benzonitrile, 17d
5-(3-acetylphenyl)-7,8-dimethoxy-1-methyl-1,3-dihydro-1,4-benzodiazepin-2-one, 17e
5-(4-isoquinolinyl)-7,8-dimethoxy-1-methyl-1,3-dihydro-1,4-benzodiazepin-2-one, 17f
7,8-dimethoxy-5-(3-hydroxymethylphenyl)-1-methyl-3-propyl-1,3-dihydro-2H-1,4-benzodiazepin-2-one, 17h
5-(3-aminophenyl)-7,8-dimethoxy-1-methyl-1,3-dihydro-2H-1,4-benzodiazepin-2-one, 17i
5-(3,4-dichlorophenyl)-7,8-dimethoxy-1-methyl-1,3-dihydro-1,4-benzodiazepin-2-one, 17j
7,8-dimethoxy-1-methyl-5-(3-methylphenyl)-1,3-dihydro-1,4-benzodiazepin-2-one, 17k.
5-(3-formylphenyl)-7,8-dimethoxy-1-methyl-1,3-dihydro-1,4-benzodiazepin-2-one, 17l
5-[3-(benzylaminomethyl)phenyl]-7,8-dimethoxy-1-methyl-1,3-dihydro-2H-1,4-benzodiazepin-2-one hydrochloride, 17m
N-[3-(7,8-dimethoxy-1-methyl-2-oxo-2,3-dihydro-1H-1,4-benzodiazepin-5-yl) phenyl]acetamide, 17n
7,8-dimethoxy-1-methyl-5-(3,4-methylenedioxyphenyl)-1,3-dihydro-2H-1,4-benzodiazepin-2-one, 17o
3-(7-hydroxy-8-methoxy-2-oxo-2,3-dihydro-1H-1,4-benzodiazepin-5-yl)benzonitrile, 22b
3-(6-bromo-7-hydroxy-8-methoxy-2-oxo-2,3-dihydro-1H-1,4-benzodiazepin-5-yl)benzonitrile, 23b
3-(9-bromo-8-hydroxy-7-methoxy-2-oxo-2,3-dihydro-1H-1,4-benzodiazepin-5-yl)benzonitrile, 23d
3-(6-bromo-7,8-dimethoxy-1-methyl-2-oxo-2,3-dihydro-1H-1,4-benzodiazepin-5-yl)benzonitrile, 24b
3-(7,8-dimethoxy-1-methyl-2-oxo-6-phenyl-2,3-dihydro-1H-1,4-benzodiazepin-5-yl)benzonitrile, 25b
3-(7,8-dimethoxy-1-methyl-2-oxo-9-phenyl-2,3-dihydro-1H-1,4-benzodiazepin-5-yl)benzonitrile, 25a
tert-butyl-3-[5-(cyanophenyl)-(7,8-dimethoxy-1-methyl-2-oxo-2,3-dihydro-1H-1,4-benzodiazepin-9-yl)phenyl]prop-2-ynylcarbamate, 25c
methyl(2E)-3-[5-(cyanophenyl)-7,8-dimethoxy-1-methyl-2-oxo-2,3-dihydro-1H-1,4-benzodiazepin-9-yl)phenyl]acrulate, 25d
tert-butyl-3-[5-(cyanophenyl)-(7,8-dimethoxy-1-methyl-2-oxo-2,3-dihydro-1H-1,4-benzodiazepin-6-yl)phenyl]prop-2-ynylcarbamate, 25e
[9-(3-aminoethynyl)-7,8-dimethoxy-1-methyl-2-oxo-2,3-dihydro-1H-1,4-benzodiazepin-5-yl]benzonitrile, 25f
[6-(3-aminoethynyl)-7,8-dimethoxy-1-methyl-2-oxo-2,3-dihydro-1H-1,4-benzodiazepin-5-yl]benzonitrile, 25g
3-(8-methoxy-2-oxo-2,3-dihydro-1H-1,4-benzodiazepin-5-yl)benzonitrile, 28a
3-(6-methoxy-2-oxo-2,3-dihydro-1H-1,4-benzodiazepin-5-yl)benzonitrile, 28b
3-(7-methoxy-2-oxo-2,3-dihydro-1H-1,4-benzodiazepin-5-yl)benzonitrile, 28c
6-methoxy-5-phenyl-1,3-dihydro-2H-1,4-benzodiazepin-2-one, 28d
7-methoxy-5-phenyl-1,3-dihydro-2H-1,4-benzodiazepin-2-one, 28e
9-bromo-7,8-dimethoxy-5-phenyl-1,3-dihydro-2H-1,4-benzodiazepin-2-one, 28f
3-(6,8-dimethoxy-2-oxo-2,3-dihydro-1H-1,4-benzodiazepin-5-yl)benzonitrile, 28g
3-(7-bromo-6,8-dimethoxy-2-oxo-2,3-dihydro-1H-1,4-benzodiazepin-5-yl)benzonitrile, 28h
3-(8-methoxy-1-methyl-2-oxo-2,3-dihydro-1H-1,4-benzodiazepin-5-yl)benzonitrile, 29a
3-(6,8-dimethoxy-1-methyl-2-oxo-2,3-dihydro-1H-1,4-benzodiazepin-5-yl)benzonitrile, 29b
3-(7-bromo-6,8-dimethoxy-1-methyl-2-oxo-2,3-dihydro-1H-1,4-benzodiazepin-5-yl)benzonitrile, 29c
3-(7,8-dimethoxy-1-methyl-2-oxo-2,3-dihydro-1H-1,4-benzodiazepin-5-yl)methyl benzoate, 34a
3-(7,8-dimethoxy-1-methyl-2-oxo-2,3-dihydro-1H-1,4-benzodiazepin-5-yl)benzoic acid, 35a
3-(7,8-dimethoxy-1-methyl-2-oxo-2,3-dihydro-1H-1,4-benzodiazepin-5-yl)N-isopropylbenzamide, 36a
N-benzyl-3-(7,8-dimethoxy-1-methyl-2-oxo-2,3-dihydro-1H-1,4-benzodiazepin-5-yl) benzamide, 36b
N-(6-amino-6-oxohexyl)-3-(7,8-dimethoxy-1-methyl-2-oxo-2,3-dihydro-1H-1,4-benzodiazepin-5-yl)benzamide, 36c
3-(7,8-dimethoxy-1-methyl-2-oxo-2,3-dihydro-1H-1,4-benzodiazepin-5-yi)-N, N-dimethylbenzamide 36d
5-{3-[(4-benzylpyperazin-1-yl)carbonyl]phenyl}7,8-dimethoxy-1-methyl-2-oxo-2,3-dihydro-1H-1,4-benzodiazepin-2-one, 36e
3-(7,8-dimethoxy-1-methyl-2-oxo-2,3-dihydro-1H-1,4-benzodiazepin-5-yl)-N-(3-phenylpropyl)benzamide, 36f.

48. The compound according to claim 36, wherein said compound is selected from the following compounds:
3-(1-benzyl-7,8-dimethoxy-2-oxo-2,3-dihydro-1H-1,4-benzodiazepin-5-yl)benzonitrile, 4m
7,8-dimethoxy-1-methyl-[5-(3-trifluoromethyl)phenyl]-1,3-dihydro-2H-1,4-benzodiazepin-2-one, 4p
3-(7,8-dimethoxy-1-methyl-2-oxo-2,3-dihydro-1H-1,4-benzodiazepin-5-yl)benzamide, 5a
3-(6-bromo-7,8-dimethoxy-1-methyl-2-oxo-2,3-dihydro-1H-1,4-benzodiazepin-5-yl)benzamide, 5b
tert-butyl-3-[3-(7,8-dimethoxy-1-methyl-2-oxo-2,3-dihydro-1H-1,4-benzodiazepin-5-yl)phenyl]propynylcarbamate, 6a
7,8-dimethoxy-5-(3′-hex-1-ynylphenyl)-1-N-methyl-1,3-dihydro-2H-1,4-benzodiazepin-2-one, 6b
6-[3-(7,8-dimethoxy-1-methyl-2-oxo-2,3-dihydro-1H-1,4-benzodiazepin-5-yl)phenyl]hex-5-ynenitrile, 6d
7,8-dimethoxy-5-(3′-hexylphenyl)-1-N-methyl-1,3-dihydro-2H-1,4-benzodiazepin-2-one, 6e
5-(4′-chloro-1,1′-biphenyl-3-yl)-7,8-dimethoxy-i -methyl-1,3-dihydro-2H-1,4-benzodiazepin-2-one, 6j
3′-(7,8-dimethoxy-1-methyl-2-oxo-2,3-dihydro-1H-1,4-benzodiazepin-5-yl)-1 ,1′-biphenyl-4-carbonitrile, 6m
3′-(7,8-dimethoxy-1-methyl-2-oxo-2,3-dihydro-1H-1,4-benzodiazepin-5-yl)-1,1 ′-biphenyl-4-carboxamide, 6n
3-(3,4-dichlorobenzyl)-1-ethyl-7,8-dimethoxy-5-phenyl-1,3-dihydro-2H-1,4-benzodiazepin-2-one, 7a
7,8-dimethoxy-1-methyl-5-[3-(4-phenyl-1,3-thiazol-2-yl)phenyl]-1,3-dihydro-2H-1,4-benzodiazepin-2-one, 9b
7,8-dimethoxy-1-methyl-5-(3-pyridyl)-1,3-dihydro-1,4-benzodiazepin-2-one, 17b.

49. A pharmaceutical composition comprising at least one compound such as defined in claim 36 and a pharmaceutically acceptable vehicle or excipient.

50. A method for treating pathologies involving the central nervous system, in particular due to a deregulation of the function of a neurotransmitter or to a deficiency in the release of a neurotransmitter, by administering to a subject in need of such treatment at least one compound such as defined in claim 36.

51. The method according to claim 50, wherein the pathology is selected in the group consisting of depression, schizophrenia, anxiety, bipolar disorder, attention deficit disorders, sleep disorders, obsessive compulsive disorder, fibromyalgia, Tourette's syndrome, pharmacodependence, epilepsy, Alzheimer's disease, Parkinson's disease, amyotrophic lateral sclerosis, multiple sclerosis, obesity and Lewy body dementia.

52. A method for treating pathologies involving the peripheral nervous system and peripheral organs in general, in particular pathologies of the type reduced natriuria, acute renal failure, hepatic dysfunction, acute hepatic failure, in particular due to age, and pathologies due to or involving dysfunctions of prolactin secretion, such as restless legs syndrome, rheumatismal, allergic or auto-inflammatory disorders, such as rheumatoid arthritis, rhinitis and asthma, by administering to a subject in need of such treatment a compound such as defined in claim 36.

53. A method for treating disorders of the central or peripheral nervous system, of chronic or acute nature, by administering to a subject in need of such treatment a compound such as defined in claim 36.

54. A method for treating memory impairment or cognitive impairment, in particular mild cognitive impairment, for treating neurodegenerative diseases, or for treating obesity, by administering to a subject in need of such treatment a compound such as defined in claim 36.
